SEQUENCE LISTING (63 sequences)

SEQ ID NO: 1 - protein, 8 aa, synthetic construct
  VARIANT 4: note = X is I or A
  VARIANT 6: note = X is G or N
  VARIANT 7: note = X is D or N
GYSXTXXY

SEQ ID NO: 2 - protein, 7 aa, synthetic construct
  VARIANT 6: note = X is G or A
ITYSGXT

SEQ ID NO: 3-5 - skipped (3 entries)

SEQ ID NO: 6 - protein, 9 aa, synthetic construct
  VARIANT 1: note = X is V or L
  VARIANT 3: note = X is S or Y
  VARIANT 4: note = X is I or D
  VARIANT 5: note = X is H or E
  VARIANT 6: note = X is V or Y
XQXXXXPYT

SEQ ID NO: 7 - protein, 8 aa, Rattus norvegicus
GYSITGDY

SEQ ID NO: 8 - protein, 7 aa, Rattus norvegicus
ITYSGGT

SEQ ID NO: 9 - protein, 9 aa, Rattus norvegicus
AKYGDGGRD

SEQ ID NO: 10 - protein, 11 aa, Rattus norvegicus
QSLSYSDGNTY

SEQ ID NO: 11 - skipped

SEQ ID NO: 12 - protein, 9 aa, Rattus norvegicus
VQSIHVPYT

SEQ ID NO: 13 - protein, 219 aa, Rattus norvegicus
DIVMTQTPPTLSASIGQSVSISCRSGQSLSYSDGNTYLNWFLQRPGQPPQPLIYLVSRLESGVPNRFSGSGSGTDFTLKISGVEAKDLGIYYCVQSIHVPYTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 444 aa, Rattus norvegicus
EVQLKESGPGLVKPSQSLFLTCSVTGYSITGDYWAWIRKFPGNKMEWIGHITYSGGTRFNPSLKSRISIKRDTSKNQFFLQLNSVTTEDTATYYCAKYGDGGRDWGQGLMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 576 aa, synthetic construct
EVQLKESGPGLVKPSQSLFLTCSVTGYSITGDYWAWIRKFPGNKMEWIGHITYSGGTRFNPSLKSRISIKRDTSKNQFFLQLNSVTTEDTATYYCAKYGDGGRDWGQGLMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGGSGGSGGGGSGDVQLQESGGGLVQAGGSLRLSCAASGFIFSSYAMGWYRQTPGKLRELVARISSGGSTWYAESVKGRFTISRDNAKNTVFLQMSSVKPEDTSAYYCNKVPYERSDYWGQGTQVTVSSA

SEQ ID NO: 16 - protein, 701 aa, synthetic construct
EVQLKESGPGLVKPSQSLFLTCSVTGYSITGDYWAWIRKFPGNKMEWIGHITYSGGTRFNPSLKSRISIKRDTSKNQFFLQLNSVTTEDTATYYCAKYGDGGRDWGQGLMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 17-19 - skipped (3 entries)

SEQ ID NO: 20 - protein, 128 aa, synthetic construct
  source 1..128: note = new world camelid
AVQLVESGGGLVQAGGSLRLSCAASGFSFDDYALGWFRQAPGKEPEGLSCISSSDGSTWYADSVKGRFTISSDNAKNTVYLQMNSLKPEDTAVYYCSAIYRLSCSVVRPTIRYALDYWGQGTQVTVSS

SEQ ID NO: 21 - protein, 8 aa, synthetic construct
  source 1..8: note = new world camelid
GFTLDDYA

SEQ ID NO: 22 - protein, 8 aa, synthetic construct
  source 1..8: note = new world camelid
ISSSDGST

SEQ ID NO: 23 - protein, 21 aa, synthetic construct
  source 1..21: note = new world camelid
SAIYRLSCSVVRPTIRYALDY

SEQ ID NO: 24 - protein, 126 aa, synthetic construct
  source 1..126: note = new world camelid
AVQLVDSGGGLVQAGGSLRLSCAASGFTFDDYAIGWFRQAPGKEPEGILCISSSDGSAYVRDSVKGRFTMSSDNAKNTMYLQMNSLRTEDTAVYHCAVATGSCPADGGQKIFLEVWGQGTQVTVSS

SEQ ID NO: 25 - protein, 8 aa, synthetic construct
  source 1..8: note = new world camelid
GFTFDDYA

SEQ ID NO: 26 - protein, 8 aa, synthetic construct
  source 1..8: note = new world camelid
ISSSDGSA

SEQ ID NO: 27 - protein, 19 aa, synthetic construct
  source 1..19: note = new world camelid
AVATGSCPADGGQKIFLEV

SEQ ID NO: 28 - protein, 15 aa, synthetic construct
GSGGGSGGSGGGGSG

SEQ ID NO: 29 - protein, 128 aa, synthetic construct
  source 1..128: note = new world camelid
EVQLVESGGGLVQAGGSLRLSCAVSGFAFEDSAIGWVRQAPGKEREAVSCISSSDGSTYYADSVKGRFTISSDNAKNTVYLQMNSLKPEDTAVYYCTADTDVSGCPAGPDEVRYLEVWGQGTQVTVSS

SEQ ID NO: 30 - protein, 127 aa, synthetic construct
  source 1..127: note = new world camelid
AVQLVESGGGLVQAGGSLRLSCAASGRTLNTLTMGWFRQAPGKEREFVCANTWNGDNTVCADSVNGRFTISRDNADNTAYLQMNSLKPEDTAIYYCAASTYSGATISVTKRYQVPYWGQGTQVTVSS

SEQ ID NO: 31 - protein, 127 aa, synthetic construct
  source 1..127: note = new world camelid
QVQLVESVGGLVQAGGSLRLSCAASGRTLDTLTMGWFRQAPGKEREFVCANTWNGGNTVCADSVNGRFTISRDNADNTAYLQMNDLKPEDTAIYYCAASTYSGATISVTRRYQVPYWGQGTQVTVSS

SEQ ID NO: 32 - protein, 125 aa, synthetic construct
  source 1..125: note = new world camelid
EVQLVESGGGSVQAGGSLRLSCATSGFRFDDYSIGWFRQAPGKEREGISCLSSSDGSSYYADSVKGRFTMSSDNAKNMMYLLMNSLKPEDTAVYYCSIARGTCPNGPTPVYLEAWGQGTLVTVSS

SEQ ID NO: 33 - protein, 125 aa, synthetic construct
  source 1..125: note = new world camelid
QVKLEESGGGLVQAGGSLRLSCATSGFRFDDYAIGWFRQAPGKEREGISCLSSSDGSSYYADSVKGRFTMSSDNAKNMMYLLMNSLKPEDTAVYYCSIARGTCPDGPTPVYLEAWGQGTLVTVSS

SEQ ID NO: 34 - protein, 127 aa, synthetic construct
  source 1..127: note = new world camelid
AVQLVDSGGGLVQAGGSLRLSCAASGRTLNTLTMGWFRQAPGKEREFVCASTWNGGNTVCADSVNGRFTISRDNADNTAYLQMNSLKLEDTAIYYCAASTYSGATIDITRRYQVPYWGQGTQVTVSS

SEQ ID NO: 35 - protein, 127 aa, synthetic construct
  source 1..127: note = new world camelid
QVQLVQSGGGLVQPGGSLRLSCAASGRTLNTLTMGWFRQAPGKEREFVCASTWNGDNTVCADSVNGRFTISRDNADNTAYLQMNSLKLEDTAIYYCAASTYSGATIDVTRRYQVPYWGQGTQVTVSS

SEQ ID NO: 36 - protein, 127 aa, synthetic construct
  source 1..127: note = new world camelid
AVQLVDSGGGLVQAGGSLRLSCAASGFNLDDYAMGWFRQAPGKEAEGISCFSSSDGSTYYADSVKGRFTISSDNAKNTVYLQMNSLKPEDTAVYHCAAAAISTCPTDARSIITLEVWGQGTLVTVSS

SEQ ID NO: 37 - protein, 128 aa, synthetic construct
  source 1..128: note = new world camelid
QVQLVESGGGLVQAGGSLRLSCAPSGFTFDDYAIGWVRQAPGKEREGVSCISSSDGSTYYADSVKGRFTISRDNAKNTVYLQMNSLKPEDTAVYYCAADLTDSSCPRSNQEHRYLEVWGQGTLVTVSS

SEQ ID NO: 38 - protein, 127 aa, synthetic construct
  source 1..127: note = new world camelid
DVQLVESGGGLVQAGGSLRLSCAASGRTLNTLTMGWFRQAPGKEREFVCASTRNGGNTVCADSVNGRFTISRDNADNTAYLQMNSLKLEDTAIYYCAASTYSGATIDVTRRYQVPYWGQGTQVTVSS

SEQ ID NO: 39 - protein, 127 aa, synthetic construct
  source 1..127: note = new world camelid
QVQLVESGGGLVQAGGSLRLSCAASGRTLNTLTMGWFRQAPGKEREFVCASTWNGGNTVCADSVNGRFTISRDNADNTAYLQMNSLKLEDTAIYYCAASTYSGATIDVTRRYQVPYWGQGTQVTVSS

SEQ ID NO: 40 - protein, 127 aa, synthetic construct
  source 1..127: note = new world camelid
DVQLVESGGALVQAGGSLRLSCAASGFTFDDYAIGWFRQAPGKEPEGVSCLSSSDGSTYYAHSVKGRFTISTDNAKNSMYLQMNSLKPEDTAVYYCSVARISSCPTNGRTKIHLDLWGQGTQVTVSS

SEQ ID NO: 41 - protein, 127 aa, synthetic construct
  source 1..127: note = new world camelid
QVQLVESGGGLVQAGGSLRLSCAASGFNLDDYAMGWFRQAPGKEREGISCISSSDGSTYYRDSVKGRFTISSDNAKNTVYLQMNSLKPEDTAVYHCAAAAISSCPTDGESIITLRVWGQGTLVTVSS

SEQ ID NO: 42 - protein, 127 aa, synthetic construct
  source 1..127: note = new world camelid
EVQLVESGGGLVQAGGSLRLSCAASGFTFDDYAMGWFRQAPGKEPEGVSCLSSSDGSTYYADSVKGRFTISSDNAKNMVYLQMNKMKPEDTAVYYCSAAAISSCPTDGRSKINLEVWGQGTQVTVSS

SEQ ID NO: 43 - protein, 127 aa, synthetic construct; identical to SEQ ID NO: 39
  source 1..127: note = new world camelid

SEQ ID NO: 44 - protein, 127 aa, synthetic construct
  source 1..127: note = new world camelid
DVQLVESGGGLVQAGGSLRLSCAASGFTFDDYAIGWFRQAPGKEREGVTCINSRDGSTYYADSVKGRFTISSDNAKNTVYLQMNSLKPEDTAVYYCAIAAVKCAEVSATTVVTLDAWGQGTQVTVSS

SEQ ID NO: 45 - protein, 127 aa, synthetic construct
  source 1..127: note = new world camelid
QVQLVESGGGLVQAGGSLRLSCAASGFNIDDYAMGWFRQAPGREREGISCFSSSDGSTFYADSVKGRFTISSDSAKNTVYLQTNSLKPEDTAVYHCAAARISSCPTDGRSIITLEVWGQGTLVTVSS

SEQ ID NO: 46 - protein, 126 aa, synthetic construct
  source 1..126: note = new world camelid
QVQLVESGGGLVQTGGSLRLSCAASGFTIDDYAMGWFRQAPGEEPEGILCISRRDGSTYYRDSVKGRFTMSTDNAKNTVYLQMTSLKPEDTAVYHCAAATGSCPTDGRTKVRLEVWGQGTLVTVSS

SEQ ID NO: 47 - protein, 127 aa, synthetic construct
  source 1..127: note = new world camelid
QVKLEESGGGLVQAGGSLRLSCAVSGFNLDDYAMGWFRQAPGKEREGVSCLSSSDGSTYYADSVKGRFTISSDNAKNTVYLQMNSLKPEDTAVYYCAAAAISSCPTNSRSIITLEVWGQGTLVTVSS

SEQ ID NO: 48 - protein, 128 aa, synthetic construct
  source 1..128: note = new world camelid
AVQLVESGGGLVQAGGSLRLSCAASGFTFDDYALGWFRQAPGKEREGVSCISSSDNSTYYADSVKGRFTISSDKSKNTVYLQMNSLKPEDTAVYYCSAIYSLSCSVVRPTTRYDLDYWGQGTQVTVSS

SEQ ID NO: 49 - protein, 127 aa, synthetic construct
  source 1..127: note = new world camelid
EVQLVESGGGLVQAGGSLRLSCAASGRTLNTLTMGWFRQAPGKEREFVCASTWSGGNTVCADSVNGRFTISRDNADNTAYLQMNSLKLEDTAIYYCAASTYSGATIDVTRRYQVPYWGQGTQVTVSS

SEQ ID NO: 50 - protein, 126 aa, synthetic construct
  source 1..126: note = new world camelid
QVQLVESGGGLVQAGGSLRLSCAASGFTFDDYAMGWFRQAPGKEPEGILCSSSSDGSTYTRDSVKGRFTISSNNAKNTLYLQMNSLKPEDTAVYHCAAATSSCPTDGGSKINLEVWGQGTLVTVSS

SEQ ID NO: 51 - protein, 128 aa, synthetic construct
  source 1..128: note = new world camelid
QVQLVESGGGLVQAGGSLRLSCAASGFTFDDYTIGWVRQAPGKELEGVSCISSNDGSTYYADSVKGRFTISSDNAKNTVYLQMNSLKPEDTAVYYCAADTDVGVCPVGPDDVRYLEVWGQGTLVTVSS

SEQ ID NO: 52 - protein, 126 aa, synthetic construct
  source 1..126: note = new world camelid
QVKLEESGGGLVQVGGSLRLSCVGFGITFDDYAMGWFRQAPGKEPEGISCISSSDGSTYAADSVKGRFTVSSDSAKNTMYLQMNSLKPDDTAVYHCAAKIGSCPTDGGSKINLEVWGQGTLVTVSS

SEQ ID NO: 53 - protein, 128 aa, synthetic construct
  source 1..128: note = new world camelid
QVKLEESGGGLVQAGGSLRLSCAASGFTFDDYAIGWFRQAPGKEREGVTCISSSDDSTYYADSVKGRFTISSDNAKNTVYLQMNSLKAEDTAVYYCAVVAAVKCAQVSPTTVYALDAWGQGTLVTVSS

SEQ ID NO: 54 - protein, 126 aa, synthetic construct
  source 1..126: note = new world camelid
AVQLVESGGGLVQAGGSLRLSCAASGFTFDDYAMGWFRQAPGKEPEGILCISSSDGSTYTADSVKGRFTISSDNAKNMVYLQMNSLKPEDTAVYHCAAAASSCPTDGGTKINLEVWGQGTLVTVSS

SEQ ID NO: 55 - protein, 126 aa, synthetic construct
  source 1..126: note = new world camelid
AVQLVDSGGGMVQAGGSLTLSCAASGFTWDDYTIGWFRQAPGKEREGVSCISSWDGSTYYADSVKGRFTISSDSAKNTVYLQMNSLKPEDTAVYYCAVGLTRTCPRRPVPVHLAAWGQGTQVTVSS

SEQ ID NO: 56 - protein, 126 aa, synthetic construct
  source 1..126: note = new world camelid
DVQLVESGGGLVQAGGSLRLSCAASGFTFDDHAIGWFRQAPGKEPEGVTCISSSDGGTYYADSVKGRFTISSDNAKNTVYMQMNSLKPEDTAVYYCAVGVAPTCPRHPVPTRLLHWGQGTQVTVSS

SEQ ID NO: 57 - protein, 126 aa, synthetic construct
  source 1..126: note = new world camelid
QVQLVESGGGLVQAGGSLRLSCAASGFTFDDYAMGWFRQAPGKEPEGILCISISDGSTYTRDSVKGRFTISSDNAKNTVYLQMNSLKPEDTAVYHCAAATGSCPTDGESKINLEVWGQGTQVTVSS

SEQ ID NO: 58 - protein, 126 aa, synthetic construct
  source 1..126: note = new world camelid
AVQLVDSGGGLVQAGGSLRLSCAASGFTFDDYAIGWFRQAPGKEPEGISCISSVDGSTYSADSVKGRFTISSDNAKNTMYLQMDSLKPEDTAVYYCAAATGSCPTDGGQKVNLEVWGQGTLVTVSS

SEQ ID NO: 59 - protein, 126 aa, synthetic construct
  source 1..126: note = new world camelid
EVQLVESGGGLVQAGGSLRLSCAASGFDFDDYAIGWFRQAPGKEPEGISCISSSDGSTYAADSVKGRFTISSDNAKNTMYLQMNSLKPEDTAVYHCAAATGSCPTDGGTKINLEVWGQGTQVTVSS

SEQ ID NO: 60 - protein, 448 aa, Homo sapiens
EVQLVQSGAEVKKPGASVKVSCKASGYSFTDYYMHWVRQAPGQGLEWIGRINPYNGAAFYSQNFKDRVTLTVDTSTSTAYLELSSLRSEDTAVYYCAIERGADLEGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 61 - protein, 218 aa, Homo sapiens
DIQMTQSPSSLSASVGDRVTITCRASQSVSTSSYNYMHWYQQKPGKPPKLLIKYASNLESGVPSRFSGSGSGTDFTLTISSLQPEDFATYYCQHSWEIPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 451 aa, synthetic construct
QVQLVESGGGLVKPGGSLRLSCAASGFTFSDYAMSWIRQAPGKGLEWVASINIGATYAYYPDSVKGRFTISRDNAKNSLYLQMNSLRAEDTAVYYCARPGSPYEYDKAYYSMAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 63 - protein, 214 aa, synthetic construct
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0312468 A1
(43) Pub. Date: Oct. 9, 2025

(54) ANTI-PROTAC ANTIBODIES AND COMPLEXES

(71) Applicant: Merck Patent GmbH, Darmstadt (DE)

(72) Inventors: Marcel RIEKER, Darmstadt (DE); Sebastian Jaeger, Darmstadt (DE); Nicolas Rasche, Darmstadt (DE); Doreen Koenning, Darmstadt (DE); Christian Schroeter, Darmstadt (DE); Hendrik Schneider, Darmstadt (DE)

(73) Assignee: Merck Patent GmbH, Darmstadt (DE)

(21) Appl. No.: 18/575,879

(22) PCT Filed: Jul. 1, 2022
(86) PCT No.: PCT/EP2022/068347

§ 371 Date: Jan. 1, 2024

(30) Foreign Application Priority Data
Jul. 2, 2021 (EP) 21183460.1

(51) Int. Cl. A61K 47/68; A61P 35/00; C07K 16/28; C07K 16/30

(52) U.S. Cl. CPC A61K 47/6803; A61K 47/6849; A61K 47/6851; A61P 35/00; C07K 16/2803; C07K 16/30; C07K 2317/31; C07K 2317/569; C07K 2317/622; C07K 2317/92

(57) ABSTRACT

Mono or bi-specific antibodies, antibody fragments, or fusion proteins thereof, are capable of binding to the VHL ligand degrading moiety (degron) of a proteolysis targeting chimera (PROTAC) and, optionally, to a target protein. Complexes (PAX) of such antibodies, antibody fragments, fusion proteins thereof, and PROTACS are also capable of. Methods for their production can be performed, and these antibodies have medical and non-medical uses.

### CROSS-REFERENCE TO RELATED APPLICATIONS

This application is a National Stage entry under § 371 of International Application No. PCT/EP2022/068347, filed on Jul. 1, 2022, and which claims the benefit of priority to European Patent Application No. 21183460.1, filed on Jul. 2, 2021. The content of each of these applications is hereby incorporated by reference in its entirety.

### REFERENCE TO A SEQUENCE LISTING

The present application is accompanied by an XML file as a computer readable form containing the sequence listing entitled, “005895USPCT SL.xml”, created on Jul. 31, 2024, with a file size of 61,252 bytes, the content of which is hereby incorporated by reference in its entirety.

### BACKGROUND

### Field of the Invention

The present invention relates to mono or bi-specific antibodies, or antibody fragments or fusion proteins thereof, capable of binding to a VHL ligand degrading moiety (degron) of a proteolysis targeting chimera (PROTAC) and, optionally, to a target protein. The invention also relates to complexes (PAX) of such antibodies, or antibody fragments or fusion proteins thereof, and PROTACS, as well as methods for their production, and medical as well as non-medical uses each thereof.

### Description of Related Art

### Degradation of Unwanted Proteins—PROTAC's

Cell maintenance and normal function requires controlled degradation of cellular proteins. For example, degradation of regulatory proteins triggers events in the cell cycle, such as DNA replication, chromosome segregation, etc. Accordingly, such degradation of proteins has implications for the cell's proliferation, differentiation, and death. While inhibitors of proteins can block or reduce protein activity in a cell, protein degradation is another possibility to reduce activity or remove the target protein completely. Utilizing a cell's protein degradation pathway can, therefore, provide a means for reducing or removing protein activity. One of the cells major degradation pathways is known as the ubiquitin-proteasome system. In this system, a protein is marked for proteasomal degradation by an E3 ubiquitin ligase that binds to the protein and transfers ubiquitin molecules to the protein. The E3 ubiquitin ligase is part of a pathway that includes E1 and E2 ubiquitin ligases, which make ubiquitin available to the E3 ubiquitin ligase catalyzed transfer to the protein. To harness this degradation pathway for a desired protein, PROTACs have been developed. PROTACs can bring the E3 ubiquitin ligase in proximity with the desired protein so that it is ubiquitinated and marked for degradation. PROTACs are heterobifunctional molecules comprising a structural motif that binds to an E3 ubiquitin ligase and another motif that binds to the protein one wishes to degrade. These groups are typically connected with a linker.

Only a small fraction of the ˜600 E3 ligases has been successfully applied for targeted protein degradation, namely MDM2, inhibitor of apoptosis protein (IAP), HECT and RBR family members, RNF4, DCAF16, EAP1-Nrf2, Von-Hippel-Lindau (VHL) and Cereblon (CRBN) with the last two playing the biggest role. VHL is a well-established E3 ligase substrate receptor which tightly binds hydroxylated HIF-1α. Based on the peptide structure around the hydroxylpropyl binding site of HIF-1α, more drug like small molecule ligands were derived that have been successfully applied to create chimeric protein degraders (Shanique, A. and Crews, C., J. Biol. Chem 296 (2021) 100647).

A widely applied ligand is VH032 (FIG. 1A)—a VHL ligand binding with strong affinity to VHL (Galdeano, C. et al. J. Med. Chem. 57 (2014) 8657-8663). It paved the way for development of additional VHL ligands like VH298 (Soares, P. et al. J. Med. Chem. 61 (2018) 599-618) since VH032 tolerates substitutions especially of the acetyl group (Ciulli, A and Ishida, T. SLAS 7Discovery 26 (2021) 484-502).

With the elucidation of the mode of action of thalidomide as CRBN ligand, CRBN became accessible for application in targeted protein degradation. Various target proteins have already been degraded by engagement of CRBN (Shanique, A. and Crews, C., J. Biol. Chem. 296 (2021) 100647).

By engagement of the aforementioned E3 ligases using chimeric degraders, targeted protein degradation has already been achieved for a plethora of proteins. Examples include: CRBN, VHL, Tau, DHODH, FKBP12, AR, ERα, RAR, CRABP-II, ALK, CK2, CDK8 and CDK9, BTK, PI3K, TBK1, FLT3, BTK, RTKs such as EGFR, HER2 and cMET, ERK1 and ERK2, BCR-ABL, RIPK2, BCL6, PCAF/GCN5, BRD4 and HDAC6, TRIM24, SIRT2, BRD9 (Scheepstra, M., Comput. Struct. Biotec. 17 (2019) 160-176; US 2018/0125821; US 2015/0291562; US 2017/0065719 A1).

Synthetically, there are many strategies for the assembly of heterobifunctional degraders. In one example (US 2017/0065719 A1), degraders were synthesized mainly by condensation reactions of activated carboxyl functionalities with an amide. Therefore, the VHL ligand VH032 and derivatives thereof were reacted with an activated carboxylic acid containing linker structure. The linker structure carried a terminal amine, which, after deprotection, was reacted with the activated carboxylic acid function of the protein binder. However, the synthesis strategies are highly dependent on the chemical nature of the ligands that have to be modified. In another example, the hydroxyl group of 7-hydroxy-thalidomide was modified using an alkylation reaction using propargyl bromides or propargyl-tosylates. The resulting compounds carried a click chemistry handle which were subsequently used to obtain full degraders by copper (I)-catalyzed azide alkyne cycloaddition (Wurz, R. P. et al. J. Med. Chem. 61 (2018) 453-461).

Androgen receptor degrading ARV-110 and estrogen receptor degrading ARV-471 (Arvinas, Inc.) are the two most advanced PROTACs in clinical development which have recently reached phase II. However, several heterobifunctional degraders have already reached phase I clinical development for a variety of targets such as the PROTAC DT2216 degrader of BCL-XL (Dialectic, Inc.) and the IRAK4 degrader KT474 (Kymera/Sanofi S.A.).

Although numerous reported PROTACs are highly efficient degraders, they are generally not tissue-specific, since they exploit E3 ligases with broad expression profiles. Tissue-specific degradation could enable optimization of the therapeutic window and minimize side effects for broad-spectrum PROTACs, increasing their potential as drugs or chemical tools. However, PROTACs exploiting E3 ligases with restricted tissue distribution have not been reported to date, and the development of novel E3 ligase ligands remains a significant challenge (Maneiro, M. et al. ACS Chemical Biology 5 (2020) 1306-1312). Another challenge in PROTAC development is their short circulatory half-life in the range of few hours in mice (Pillow, T. H. et al., ChemMedChem 15 (2020) 17-25; Burslem, G. M. et al., J. Am. Chem. Soc. 140 (2018) 16428-16432).

Additionally, the efficacy of PROTACs is often hampered by their low permeability (Klein, V. G. et al., ACS Med. Chem. Lett. 11 (2020) 1732-1738) which limits their ability to enter cells and induce protein degradation.

Therefore, there is an ongoing need in the art for enhanced and targeted delivery of PROTACs to cells that contain the to-be-degraded protein target.

To address this need, there have been attempts to enhance delivery of PROTACs to particular cells by using covalent antibody-PROTAC conjugates similar to antibody-drug conjugates (ADCs). Such constructs make use of the cell-target selective binding and enhanced pharmacokinetics conferred by the antibody.

### Targeted Drug Delivery—Antibody-Drug Conjugates (ADCs)

The basic concept of ADCs is rather simple. Prerequisite is an antigen that allows discrimination between, e.g., cancer and healthy cells on a molecular basis. This can, for example, be a certain cell surface receptor, which is heavily upregulated in tumor cells. An antibody against such an antigen can serve as a targeting vehicle for a highly potent cytotoxic agent—the “payload”. To form the ADC, the cytotoxic agent needs to be covalently attached to the antibody via a linker that is stable in the circulation to avoid premature release of the payload. After administration, the ADC distributes throughout the body of the patient and binds to its antigen on the surface of tumor cells. The antibody-antigen complex is then internalized by the cell and directed to the lysosome via endogenous intracellular trafficking pathways. After reaching the lysosome, the ADC gets degraded and thereby releases its toxic cargo. The free toxin can then bind to its intracellular target and, thus, induce apoptosis and killing of the cancer cell. In some cases, the toxin can leave the cancer cell and act on the adjacent, ideally cancerous cells as well. This process is called the bystander effect and its extent depends on the applied linker and drug. Healthy cells, on the other hand, are mainly spared since the antibody should only bind and deliver the toxin to cancer cells that express the antigen. ADCs that have been approved for the treatment of cancer include HER2 targeting DM1 conjugate Kadcyla, Adcetris, an anti-CD30 ADC carrying the tubulin inhibitor MMAE and the CD33-targeting-Calicheamicin ADC Mylotarg.

The design of ADCs is a multidisciplinary endeavor since they are composed of biotechnologically produced biomolecules and chemically synthesized, highly potent small molecule drugs. Both entities are produced separately and combined afterward to a highly complex hybrid molecule. Hence, the entire process of ADC development starting from the design of the individual components to the final production of the conjugate comes along with significant technical challenges. According to the term “antibody-drug conjugate,” the main components of an ADC are the drug and the antibody. To couple these entities, however, a linker that connects the mAb with the drug is required. Careful selection of this linker, taking both the mAb and the payload into account, is crucial for the efficacy and safety of the final ADC. In the bloodstream, the linker should be as stable as possible to prevent premature payload release which could otherwise cause systemic off-target toxicity. But once the ADC has reached the target cell, the payload has to be active without being hampered by an attached linker. In addition, the length and chemical nature of the linker can have strong effects on the pharmacokinetics and -dynamics of ADCs. Linkers utilized for ADCs are mainly categorized into non-cleavable and cleavable ones. Non-cleavable linkers are stable both in the circulation and in cells, whereas cleavable linkers are designed to be degraded by specific intracellular mechanisms within the target cell. It becomes clear from the above, that engineering the appropriate linker for a given ADC is a challenge in its own right.

While all three parts of an ADC—the antibody, the linker, and the cytotoxic payload-determine the key properties of the final conjugate, a similarly important parameter is the way these components are assembled. The linker and the payload are produced by chemical synthesis either as a combined linker-payload structure that is directly conjugated to the mAb or as individual components that are successively assembled during ADC generation. In both cases, a small molecule needs to be conjugated to a mAb without impairing its favorable properties, which is a major technical challenge. The main parameters that need to be controlled during ADC generation are the number of linker-drugs conjugated to each antibody, termed drug-to-antibody ratio (DAR), and the positions on the antibody surface the structures are attached to (conjugation sites). Both parameters can decisively influence several properties of an ADO including its stability and pharmacokinetic behavior and ultimately also its toxicity and efficacy profile. On the one hand, warheads used for ADCs are mostly hydrophobic and an increasing DAR can significantly alter the overall hydrophobicity and severely disturb protein stability of the final conjugate. On the other hand, a certain amount of drug, depending on its potency, is required to reach a sufficiently active ADC. However, not only the DAR but also the conjugation site and chemistry heavily impact these parameters. For instance, several studies have shown, that certain sites show superior tolerance toward challenging payloads and result in more stable conjugates than others by providing a favorable microenvironment and steric shielding on the antibody surface. Hence, finding a favorable combination of the individual components linker, drug and mAb as well as a suitable DAR, conjugation strategy and conjugation sites is key for the development of efficient and safe therapeutics (Dickgiesser, S. et al., Introduction to Antibody Engineering, Springer (2021) 189-214).

ADCs with PROTACs as Payloads

A special shape of ADCs are Degrader-ADCs where the drug is represented by a protein degrader. Here, a linker needs to be attached to the degrader to facilitate conjugation to the antibody. Besides choosing the right linker, it is also crucial to identify a suitable attachment site on the degrader-either in the warhead, degron or linker part. Several publications have proven the feasibility of this concept.

One example are estrogen receptor a (ERα) degraders that were covalently attached to a HER2-targeting antibody via conjugation to engineered cysteines. Therefore, the degrader had to be chemically modified with a protease cleavable linker on either the ERα-targeting moiety or on the XIAP binder. In case of the degrader-ADC where the linker was attached at the warhead, ERα degradation was achieved in HER2 overexpressing MCF7 cells while significantly less degradation was observed in parental MCF7 cells. Additional linker options were tested. The hydroxyl group of the hydroxyprolyl residue of the VHL ligand was modified with a carbonate linker which was conjugated via an activated disulfide to an HER2 antibody. Additionally, a diphosphate containing linker was attached to the hydroxyprolyl residue of the VHL ligand. In both cases, the conjugates lacked selectivity (Dragovich, P. S. et al., Bioorg. Med. Chem. Lett. 30 (2020) 126907).

Besides ERα as intracellular PROTAC target for degrader-ADCs, BRD4 was intensively studied as target protein, too. One example shows the selective delivery of a BRD4 degrader to HER2-positive cells leading to BRD4 degradation via an HER2-targeting antibody. The degrader was conjugated via a combination of cysteine conjugation and click chemistry using an acid-cleavable ester linkage at the hydroxyprolyl residue of the VHL ligand (Maneiro, M. et al. ACS Chemical Biology 5 (2020) 1306-1312). In another example the BRD4 degrader GNE987 was conjugated to engineered cysteines of a CLL1-targeting antibody reaching a DAR of 6. The PROTAC was therefore modified with an acid-cleavable carbonate linker comprising an activated disulfide for conjugation. The conjugate significantly improved the pharmacokinetic profile of the PROTAC and the in vivo efficacy in a mouse xenograft model while being well tolerated (Pillow, T. H. et al., ChemMedChem 15 (2020) 17-25).

BRD4 degrader conjugates have been investigated in depth by this group in two additional publications (Dragovich, P. S. et al., J. Med. Chem. 64 (2021) 2534-2575; Dragovich, P. S. et al., J. Med. Chem. 64 (2021) 2576-2607). Multiple conjugates of BRD4 degraders have been prepared based on STEAP1 and HER2 antibodies. The focus of the work was the investigation of the ideal linker connecting ADC and degrader as well as the ideal attachment point of this linker on either target protein ligand, E3 ligase ligand or the linker between target protein ligand and E3 ligase ligand. Therefore, several target protein ligands were evaluated including JQ1 derivatives incorporating a suitable chemical handle for linker attachment. In case of the linker between target protein and E3 ligase ligand, multiple variants were tested including PEG and aliphatic chains as well as versions with incorporated chemical handles for linker attachment. Furthermore, derivatives of the VHL ligand were evaluated that were chemically modified to allow linker attachment. The conjugates were able to induce receptor-selective protein degradation, but only a few displayed selective cytotoxicity. Those publications highlight the complexity of conjugation of chimeric degraders to antibodies. For the mentioned degrader-conjugates two patent applications were filed (WO 2020/086858; WO 2017/201449).

In addition to that, BRD4 degrader conjugates have also been found in patent literature targeted to HER2 (WO2019/140003A1) and dual degraders of BRD4 and PLK1 have been investigated as payloads for CD33-targeting antibodies (WO2020/073930A1). Furthermore, TGFβR2 degraders have been conjugated to HER2 and TROP2 antibodies for targeted delivery (WO2018/227018A1; WO2018/227023A1).

### Non-Covalent Approaches of Delivering Drugs to Target Cells

Multiple approaches have been described for non-covalent drug delivery where the drug always needs to be chemically connected to a ligand or a hapten that binds to or can be bound by an antibody.

For instance, Gemcitabine was chemically modified with the affinity ligand 4-mercaptoethylpyridine that binds to several sites on the antibody. By mixing the antibody with the affinity ligand modified Gemcitabine an ADC assembled which was able to induce selective toxicity on target positive cancer cells and had a pharmacokinetic profile comparable to the unmodified antibody. Tumor regression of the Gemcitabine ADC was observed in a mouse xenograft model (Gupta, N. et al., Nat. Biomed. Eng. 3 (2019) 917-929).

Additionally, several approaches used the modification of small molecules such as the anti-cancer drug doxorubicin or the fluorophore Cy5, siRNA, proteins like GFP and Saporin with the hapten digoxigenin to facilitate cellular drug delivery (Metz, S. et al., Proc. Natl. Acad. Sci. 108 (2011) 8194-8199; Schneider, B. et al., Mol. Ther.—Nucleic Acids 1 (2012) e46; Mayer, K. et al., Int. J. Mol. Sci. 16, (2015) 27497-27507).

Furthermore, the cytotoxic drug Duocarymcin DM could be delivered to EGFR-positive cells using a bispecific antibody binding to EGFR and simultaneously to cotinine. In order to deliver Duocarmycin DM to the target cells, a peptide was synthesized carrying cotinine C- and N-terminally and 4 Duocarmycin DM molecules were attached to the peptide via a cleavable valine-citrulline linker. The construct was tested in a mouse EGFR-expressing A549 xenograft model and exceeded anti-tumor effects of an isotype control construct (Jin, J. et al., Exp. Mol. Med. 50 (2018), 67). A similar construct was used to deliver duocarmycin to mPDGFRB-positive cells (Kim, S. et al., Methods 154 (2019) 125-135).

Various other publications elaborate on the concept of complexation using hapten-modified compounds and anti-hapten antibodies (Yu, B. et al., Angew. Chemie-Int. Ed. 58 (2019) 2005-2010; Kim, H. et al., Mol. Pharm. 16 (2019) 165-172; Kilian, T. et al., Nucleic Acids Res., 47 (2019) e55).

A comparable approach uses the covalent conjugation of Tubulysin A to Fc binding proteins like protein A or G to assemble a complex with an antibody for targeted drug delivery (Maso, K. et al., Eur J Pharm Biopharm 142 (2019) 49-60).

While there are many examples for non-covalent drug delivery using haptenylated compounds together with anti-hapten antibodies or affinity ligands/proteins binding to antibodies, examples for non-covalent drug delivery using unmodified drugs are scarce.

Despite all these attempts, there is still a need for a well-defined, efficient and specific delivery platform for PROTACS with effective release of the payload at the target that can be broadly applied.

### SUMMARY OF THE INVENTION

The present invention relates to mono or bi-specific antibodies, or antibody fragments or fusion proteins thereof, capable of binding to a VHL ligand degrading moiety (degron) of a proteolysis targeting chimera (PROTAC) and, in case of bi-specific antibodies, to a target protein. The invention also relates to complexes of such antibodies, or antibody fragments or fusion proteins thereof, and PROTACS, methods for their production, as well as medical and non-medical uses each thereof. Such PROTAC-antibody complexes, are hereinafter referred to as “PAX”.

In one embodiment, the target protein is a cell surface antigen on a target cell, to which the PROTAC is delivered. Upon delivery, the PROTAC is released into the cytosol of the target cell where it binds to the degradation target protein, and thereby initiates degradation through the cellular proteasomes.

The advantage of PAX, as compared to covalently linked antibody drug conjugates (ADC's) is that no specific manufacturing step is required to link the PROTAC to the antibody. Another advantage is that, once a PAX has released its PROTAC payload, it is ready for a new cycle of PROTAC binding and targeted delivery, e.g., of a PROTAC molecule, which has left a target cell, to which it had previously been delivered.

Yet another advantage is an improved pharmacokinetics profile, in that PROTAC complexation in a PAX is expected to extend a PROTAC's half-life in a patient's body. Due to the complexation of the PROTAC with the anti-PROTAC antibody, the complex stability determines the clearance of the PROTAC. As long as the PROTAC is complexed by the antibody, it cannot be cleared renally due to the high molecular weight of the antibody.

In one embodiment the bi-specific antibody comprises a) a monospecific bivalent antibody consisting of two full length antibody heavy chains and two full length antibody light chains whereby each chain comprises only one variable domain, b) two monospecific monovalent single chain antibodies (scFv's), each consisting of an antibody heavy chain variable domain, an antibody light chain variable domain, and a single-chain-linker between said antibody heavy chain variable domain and said antibody light chain variable domain, optionally c) two or more additional copies of the scFv's (b), fused to the said scFv's, and, optionally d) peptide linkers connecting a), b), and/or c).

In one embodiment the bi-specific antibody comprises a) a monospecific bivalent antibody consisting of two full length antibody heavy chains and two full length antibody light chains whereby each chain comprises only one variable domain, b) two heavy chain single domain (VHH) antibodies, each consisting of one antibody variable domain, optionally, c) two or more additional copies of the VHH's (b), fused to the said VHH's, and, optionally d) peptide linkers connecting a), b), and/or c).

The person of skill in the art understands that the presence of a peptide linker, or its length, has no impact on the performance of the invention. However, in an embodiment, the peptide linkers consist of 1-50 amino acids, preferably 1-35, amino acids, more preferably 3-20 amino acids, and even more preferably 12-18 amino acids, for example, 15 amino acids.

In one embodiment, the peptide linkers connect the C-termini of the antibody's heavy chains and/or light chains with the N-termini of the scFv's or VHH's.

In one embodiment, the scFv's or VHH's are fused to the C-termini of the antibody's heavy chains.

In one embodiment, the antibody does not comprise additional copies of the scFv's or VHH's.

In one embodiment, the variable regions of the monospecific bivalent antibody bind to the target protein, and the scFv's or VHH's bind to the PROTAC.

In an alternative embodiment, the variable regions of the monospecific bivalent antibody bind to the PROTAC, and the scFv's or VHH's bind to the target protein.

In one embodiment, the VHL ligand is VH032, or a derivative thereof.

In one embodiment the bi-specific antibody is characterized in that the target protein is a cell surface antigen, e.g., a tumor antigen. In preferred embodiments the target protein is HER2, CD33, CLL1, EGFR, CD19, CD20, CD22, B7H3 (CD276), CD30, CD37, CEACAM5, cMET, MUC1, ROR1, CLDN18.2, TROP2, BCMA, CD25, CD70, CD74, CD79b, TROP2, cMET, STEAP1, NaPi2b, PSMA, Integrin alpha-V, FRα, MUC16, Mesothelin, CEACAM5, CanAg-MUC1 glycoform, EpCAM, HER3 or TNC. In more preferred embodiments the target protein is HER2, CD33, CLL1 or EGFR.

The person of skill in the art however understands that the invention will work with any target protein, which establishes a subset of cells for targeted PROTAC delivery, as compared to any cell present in the patient's body.

Another aspect of the invention is a method for treating a disease susceptible to the degradation of a certain target protein, wherein the PAX is administered to a patient in need thereof.

It is contemplated that the PAX disclosed herein may be used to treat various diseases or disorders. Exemplary hyperproliferative disorders include benign or malignant solid tumors and hematological disorders such as leukemia and lymphoid malignancies Others include neuronal, glial, astrocytal, hypothalamic, glandular, macrophagal, epithelial, stromal, blastocoelic, inflammatory, angiogenic and immunologic, including autoimmune disorders.

Another aspect of the invention is a pharmaceutical composition comprising the PAX according to the invention. In yet another aspect the said pharmaceutical composition is used in targeted cancer therapy.

In yet other aspects the antibody of the invention serves to detect and/or quantify PROTAC's, or to purify PROTAC's of interest, e.g., from impurities/byproducts of the manufacturing process.

### BRIEF DESCRIPTION OF THE DRAWINGS

FIGS. 1A-C: Chemical structure of VHL ligand VH032 and derivatives. (1A) Structure of VH032. (1B) Markush structure of VH032-based VHL-ligands. (1C) Representation indicating the different exit vectors (R1, R2, R3) for the linker that connects the VH032-based degron to different warheads, exemplarily shown for the MZ1, AT1 and ACBI1 warhead. MIC2 antibody tolerates exit vector R1 and R2 resulting in binding of PROTACs MZ1 and AT1.

FIG. 2A and FIG. 2B: Amino acid sequences of bispecific fusion proteins against cell surface antigen and PROTAC. Bold: Sequences of anti-PROTAC antibody MIC2, with CDR sequences underlined; Italic: Linker sequence; underlined: antibody fragment sequences (anti-EGFR VHH sequence or anti-HER2 scFv.

FIG. 3: Graphical depiction of a range of possible BsAb variants according to the invention.

FIG. 4: Chemical structures of VH032-based haptens.

FIG. 5A and FIG. 5B: Hapten-to-carrier protein ratios for cBSA and huFc and the corresponding Individual haptens derived from MALDI-MS measurements.

FIG. 6: Study plan for hybridoma screening to identify anti-VH032 antibodies.

FIG. 7: Assay principle for affinity determination. A) MIC2 is immobilized on the SPR chip. The analyte flows past the antibody and is captured. After the PROTAC is captured, the buffer is changed and the PROTAC can dissociate again. B) The association of the PROTAC with the antibody is observed as an increase in signal while the dissociation leads to a decrease in signal. This is exemplarily shown for the binding of MIC2 to PROTAC MZ1.

FIG. 8A and FIG. 8B: VH032-based PROTACs tested for binding in the SPR assay.

FIG. 9A, FIG. 9B, and FIG. 9C: Binding assessment of bispecific antibodies aEGFRxMIC2, aHER2xMIC2 in comparison to parental antibody MIC2 to several PROTACs. Affinity parameters were broken down by on- and off-rate as well as affinity.

FIG. 10: Loading-dependent complexation analyzed via SE-HPLC. The peak distribution shifts with increasing theoretical loading from the peak of the uncomplexed antibody (0% loading) (left), half-loaded (50% loading; antibody: PROTAC molar ratio=1:1) antibody, towards a peak of fully loaded (100% loading; antibody: PROTAC molar ratio=1:2) antibody.

FIG. 11: SE-HPLC profile of unpurified and purified aEGFRxMIC2+GNE987 complex. Violet: unpurified sample; cyan: Desalted sample.

FIG. 12: Peak distribution of unpurified and purified aEGFRxMIC2+GNE987 complex.

FIG. 13: Peak distribution of aEGFRxMIC2+GNE987 complex over time.

FIG. 14: Chemical structure of linker-modified GNE987.

FIG. 15: Exemplary fluorescence images of BRD4 levels. Higher green fluorescence correlates with higher BRD4 abundance. While untreated cells had the strongest fluorescence, fluorescence was reduced for cells treated with 4 nM GNE987 as well as EGFR targeting C225-L328C-GNE987 and aEGFRxMIC2 loaded with GNE987. The fluorescence was increased in comparison to the EGFR-targeting complex in case of treatment with 4 nM non-binding aHER2xMIC2 loaded with GNE987. Green fluorescence is depicted in shades of grey.

FIG. 16A and FIG. 16 (B): BRD4 level quantification. A) GNE987, C225-L328C-GNE987 and aEGFRxMIC2+GNE987 had comparable effects on BRD4 levels over the whole investigated concentration range, while aHER2xMIC2+GNE987 degraded BRD4 to a smaller extent. 16B) BRD4 degradation induced at 4 nM concentration of all analytes.

FIG. 17: Dose-response curve plot of aEGFRxMIC2+GNE987 and controls. Serial dilutions of the test compounds were added to MDAMB468 cells and after 3 days of incubation the impact on cell viability of each individual compound was assessed. While EGFR-targeting aEGFRxMIC2+GNE987 at 50% (1:1) loading as well as benchmark C225-L328C-GNE987 and GNE987 had comparable potencies, non-binding controls MIC2+GNE987 and aHER2xMIC2+GNE987 at 50% (1:1) loading had a reduced potency.

FIG. 18: Dose-response curve plot of aEGFRxMIC2+GNE987 and controls. The PROTAC GNE987 has the highest potency followed by aEGFRxMIC2+GNE987 at 25% loading. The non-binding controls MIC2+GNE987 and aHER2xMIC2+GNE987 at 50% (1:1) loading had reduced effects on cell viability.

FIG. 19: IC50-value plot for the investigated molecules in N=3 biological replicates.

FIG. 20: Dose-response curve of HEPG2 cells treated with PROTAC-ADCs and PROTAC shuttles.

FIG. 21: Molecular structures of BRD4-degrading GNE987 and its analogue GNE987P possessing a PEG linker.

FIG. 22: Complexed aEGFRxMIC2+GNE987P shows an increased cytotoxic effect on EGFR-expressing MDAMB468 cells at a concentration range 0.1-10 nM compared to the GNE987P alone, indicating targeted delivery. Complexation with non-targeting MIC2+GNE987P reduces cytotoxicity of GNE987P completely.

FIG. 23: Mouse plasma stability of GNE987 alone or in complex with aEGFRxMIC2 over 72 h.

FIG. 24: Mouse plasma stability of the bispecific antibody aEGFRxMIC2 complexed with GNE987 over 96 h.

FIG. 25: Stability assessment of the complex aEGFRxMIC2+GNE987 at 50% loading over 96 h in mouse plasma. The aEGFRxMIC2+GNE987 complex was captured on beads and the supernatant collected for LC-MS analysis of unbound GNE987. Afterwards, bead-bound aEGFRxMIC2+GNE987 complex was eluted from the beads subjected to GNE987 quantification using LC-MS.

FIG. 26: Immunization schedule for new world camelid immunization to produce anti-hapten antibodies.

FIG. 27: Biotinylated VH032 for antibody discovery via phage display.

FIG. 28: Expression rate of VHH fusion proteins versus unmodified parent antibody.

FIG. 29A and FIG. 29B: Flow Cytometric analysis of cellular binding to MV411 and MDAMB468 of CD33xMIC5 or EGFRxMIC5, respectively, compared to the parental antibody lacking the VHH MIC5.

FIG. 30: Comparison of cellular binding of CD33-binding CD33xMIC7 loaded and not-loaded with PROTAC GNE987 to CD33-expressing cell lines.

FIG. 31: Structure of pH responsive VH032-pHAb dye.

FIG. 32: Flow cytometric analysis of the internalization of CD33xMIC7 into CD33-positive cells MOLM13, MV411 and U937 and CD33-negative RAMOS cells over 6 h.

FIG. 33: Western Blot of CD33xMIC7+GNE987 (1:1) and DIGxMIC7+GNE987 (1:1) on CD33 positive MV411 cells. Concentrations above plot are given in mol/L. Size of marker (right) is given in kDa.

FIG. 34: Western Blot analysis of degradation patterns for CD33xMIC7+GNE987 (1:1) and DIGxMIC7+GNE987 (1:1) on MV411 cells.

FIG. 35: Comparison of cell viability data depending on CD33 receptor expression levels. CD33xMIC5+GNE987 at 50% loading induced cytotoxicity on CD33-positive MV411 and MOLM13 cells but had only minor effects on RAMOS cells lacking CD33.

FIG. 36: Cell cytotoxicity of CD33xMIC5 loaded with 25, 50 and 75% PROTAC GNE987 compared with cell cytotoxicity of GNE987 on CD33-positive MV411 cells.

FIG. 37A, FIG. 37B, and FIG. 37C: Cell viability data of CD33xMIC5 antibody loaded with varying amounts of PROTAC GNE987P per antibody compared to the PROTAC GNE987P alone.

FIG. 38A and FIG. 38B: Cell viability data of CD33xMIC5 antibody loaded with PROTAC FLT3d1 per antibody compared to the PROTAC GNE987P alone. Cell viability was analyzed after 6 days of treatment.

FIG. 39: Cell viability assay of CD33xMIC5+GNE987 at 75% loading (1:1.5) either pre-complexed or not-pre-complexed on CD33-positive MV411 and CD33-negative RAMOS cells. In case of the not-pre-complexed samples, antibody (CD33xMIC5) and PROTAC (GNE987) were added separately to the cell suspension as treatment. The PROTAC GNE987 was tested on the cells for reference.

FIG. 40A, FIG. 40B, and FIG. 40C: Cell viability assay of CLL1xMIC7+GNE987P and DIGxMIC7+GNE987P at 75% loading (1:1.5) on CLL1-positive MOLM13 and U937 and on CLL1-negative K562 cells. The PROTAC GNE987 alone was tested on the cells for reference.

FIG. 41A, FIG. 41B, FIG. 41C, and FIG. 41D: Cell viability assay of CLL1xMIC7+GNE987, CLL1xMIC7+GNE987P and CLL1xMIC7+SIM1 at 75% loading (1:1.5) on CLL1-positive MV411 and U937 cells and on CLL1-negative RAMOS and K562 cells. The PROTACs GNE987, GNE987P and SIM1 were tested on the cells for reference.

FIG. 42A, FIG. 42B, FIG. 42C, FIG. 42D, FIG. 42E, and FIG. 42F: Cell viability assay of B7H3xMIC7+GNE987P or B7H3xMIC7+SIM1 at 75% loading (1:1.5) on B7H3-positive MV411 and U937 cells and B7H3-negative RAMOS cells. The PROTACs alone (GNE987P and SIM1) were tested on the cells for reference.

FIG. 43A, FIG. 43B, FIG. 430, and FIG. 43D: Cell viability assay of B7H3xMIC7+GNE987 and DIGxMIC7+GNE987 at 75% loading (1:1.5) on B7H3-positive MV411 and U937 cells and B7H3-negative RAMOS cells. The PROTAC GNE987 was tested on the cells for reference.

FIG. 44A, FIG. 44B, FIG. 44C, FIG. 44D, FIG. 44E, and FIG. 44F: Cell viability assay of NAPI2BxMIC7 and DIGxMIC7 loaded with GNE987, GNE987P or SIM1 at 50% loading (1:1) on NAPI2B-positive OVCAR3 and NAPI2B-negative SKOV3 cells. The PROTACs GNE987, GNE987P, and SIM1 were tested on the cells as reference.

FIG. 45: Comparison of cell cytotoxicity of EGFRxMIC5+GNE987 loaded with 50% PROTAC and Cetuximab-based EGFR-binding PROTAC-ADC (DAR=1.62) on EGFR-negative HEPG2 and EGFR-positive MDAMB468 cells.

FIG. 46: PK study of GNE987 and MIC2+GNE987 complexes at a loading of 81.3% in female SCID Beige mice after IV administration.

FIG. 47: PK study of CD33xMIC5+GNE987 and CD33xMIC7+GNE987 PROTAC-Antibody complexes with a theoretical loading of 100% C57BL/6N mice after IV administration of 30 mg/kg. Shown is the detected concentration of GNE987.

FIG. 48: Clearances of unmodified antibody CD33 Ab, antibody-VHH fusion proteins CD33xMIC5 and CD33xMIC7 as well as CD33xMIC5 and CD33xMIC7 loaded with GNE987 in comparison.

FIG. 49: MV411 xenograft efficacy study of CD33xMIC7+GNE987 in female CB17 SCID mice. 30 mg/kg CD33xMIC7+GNE987 were given once or twice in comparison to 0.38 mg/kg GNE987 given once or twice (day 1 and 8). Additionally, efficacy of 30 mg/kg CD33xMIC5+GNE987 given once (day 1) was assessed and the effect of the antibody alone (30 mg/kg CD33xMIC7) as control.

FIG. 50A, FIG. 50B, FIG. 50C, FIG. 50D, and FIG. 50E: Antibody VHH sequences obtained from immunization of new world camelids and phage display screening.

FIG. 51: Sequence of anti-CLL1 antibody 6E7L4Hle.

### TABLE OF TABLES

Table 1: Binding epitope of the antibodies of the invention.

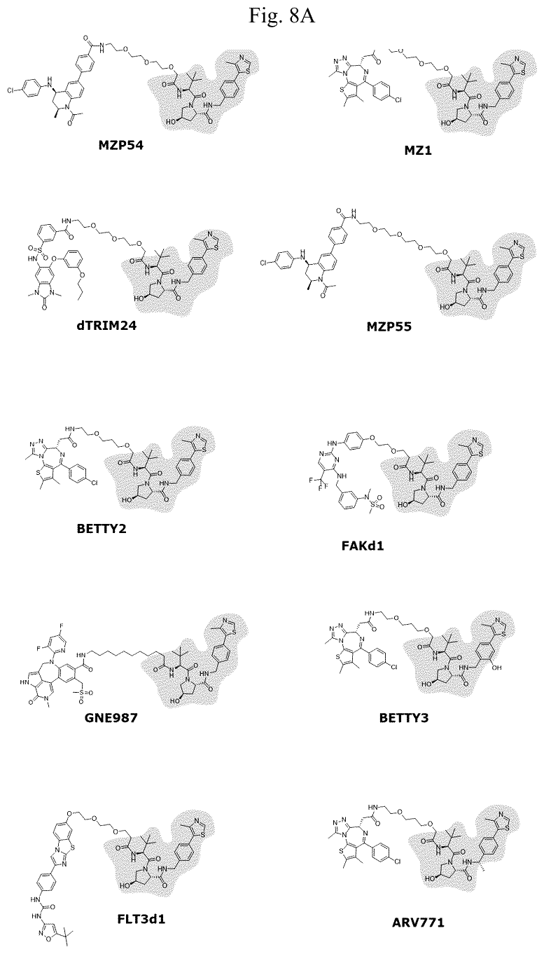

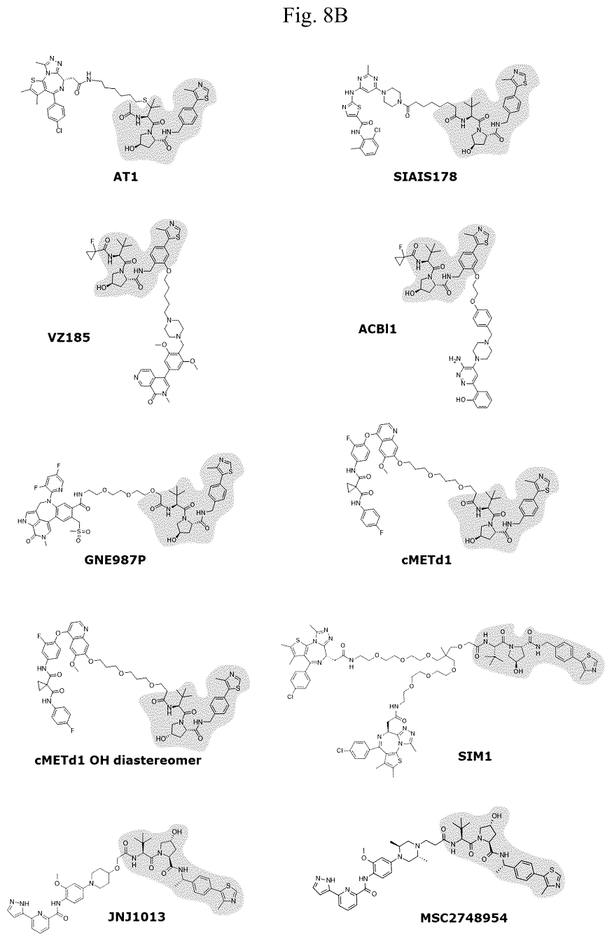

Table 2: Overview on affinity parameters KD, association rate kon, dissociation rat koff for combinations of MIC2 and PROTACs (see structures, FIGS. 8A-8B) obtained using a 1:1 kinetic binding model for MIC2 and KD for binding of PROTACs to MIC1 derived from a steady-state model. NM=Not measured; NB=No binding

Table 3: Overview on required final PROTAC concentrations to achieve desired loading

Table 4: IC50-values of EGFR-binding aEGFRxMIC2 and non-binding control MIC2 complexed with GNE987 at loadings of 25 and 50%

Table 5: IC50-values of EGFR-binding aEGFRxMIC2+GNE987 complex and controls on MDAMB468. The potencies and standard deviation are derived from three independent experiments

Table 6: Storage stability assessment of antibody-PROTAC complexes in PBS pH 6.8, 5% DMSO final

Table 7: Library characteristics for antibody hit discovery campaign using phage display Table 8: Affinities (KD) of VHH clones to PROTACs determined using SPR. The VHHs were studied as antibody fusion proteins by C-terminal addition to the heavy chain of either an anti-CD33 or anti-CLL1 antibody. N/D—not detected (complete PROTACS structures can be found in FIGS. 8A-8B)

Table 9: IgG-type antibody backbones for fusion with VHH antibody fragments Table 10: Nomenclature for PAX targeting CD33

Table 11: Cellular profiling of CD33-binding gemtuzumab (G)- and EGFR-binding cetuximab (c)-based VHH fusion proteins combined with PROTAC GNE987 on EGFR-positive MDAMB468 cells and MDAMB468-negative HEPG2 cells. IC50 values were used to calculate selectivity indices

Table 12: Primary antibodies used for Western Blot analysis

Table 13: Cellular profiling of different CD33xMIC7 combined with PROTACs GNE987 and GNE987P or PROTACs alone on CD33-positive MV411 cells and CD33-negative RAMOS cells. IC50 values are depicted in M

Table 14: Cellular profiling of PROTACs ARV771, GNE987, GNE987P and EGFR-positive cells and EGFR-negative HEPG2 cells. IC50 values are depicted in M Table 15: Cellular profiling of EGFRxMIC7 combined with PROTACs GNE987, GNE987P and SIM1 at a loading of 50% on EGFR-positive cells and EGFR-negative HEPG2 cells. As a non-internalizing control, a digoxigenin-binding DIGxMIC7 fusion protein was utilized. IC50 values are depicted in M

Table 16: Cellular profiling of EGFRxMIC7 combined with PROTACs ARV771, GNE987, GNE987P and SIM1 at a loading of 75% on EGFR-positive cells and EGFR-negative HEPG2 and EGFR-low MCF7 cells. As a non-internalizing control, a digoxigenin-binding DIGxMIC7 fusion protein was utilized

Table 17: Cellular profiling of HER2xMIC7 combined with the PROTAC GNE987, GNE987P and SIM1 at a loading of 75% on HER2-positive cells and HER2-negative MDAMB468 cells Table 18: Cellular profiling of TROP2xMIC7 combined with the PROTAC GNE987 at a loading of 75% on TROP2-positive cells and TROP2-negative SW620 cells

Table 19: Summary of pharmacokinetic parameter of CD33-based VHH-fusions with MIC5 and MIC7 loaded and unloaded with PROTAC GNE987 and the parental antibody CD33 Ab. The analytes were administered at 30 mg/kg and the PK parameters for the quantification total antibody (tAntibody) and the PROTAC GNE987 are depicted. Abbreviations: 11/2: half-life; Cmax: maximum serum concentration; AUCO-inf: Area under the curve to infinite time; Cl: Clearance; Vss: Steady state volume of distribution. SD: Standard deviation

Table 20: Summary of the scope of this work. The investigated combinations are depicted in tabular form

### DETAILED DESCRIPTION OF THE INVENTION

### Definitions

A “PROTAC” (proteolysis targeting chimera) is a heterobifunctional small molecule composed of two active domains and a linker, capable of removing specific unwanted proteins. Rather than acting as a conventional enzyme inhibitor, a PROTAC works by inducing selective proteolysis. PROTACs consist of two covalently linked protein-binding molecules: one (in many instances) capable of engaging an, and another that binds to a target protein meant for degradation. Recruitment of the E3 ligase to the target protein results in ubiquitination and subsequent degradation of the target protein by the proteasome. The concept was initially described by Deshaies and coworkers in 2001 (Skamoto, K. M. et al., Proc. Natl. Acad. Sci. USA 98 (2001) 8554-8559).

The term “antibody” includes monoclonal antibodies (including full length antibodies which have an immunoglobulin Fc region), antibody compositions with poly-epitopic specificity, multi-specific antibodies, in particular bi-specific antibodies, diabodies, and single-chain molecules (such as scFv's), single domain antibodies (nanobodies, such as VHH's derived from new world camelid species, e.g., llamas), as well as antibody fragments (e.g., Fab, F(ab′)2, and Fv).

The term “immunoglobulin” (Ig) is used interchangeably with “antibody” herein. The basic 4-chain antibody unit is a hetero-tetrameric glycoprotein composed of two identical light (L) chains and two identical heavy (H) chains. An IgM antibody consists of 5 of the basic hetero-tetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the alpha and gamma heavy chain isotypes and four CH domains for mu and epsilon heavy chain isotypes. Each L chain has at the N-terminus, a variable domain (VL) followed by a constant domain at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see e.g., Schroeder, H., Cavacini, L., J. Allergy Clin. Immunol. 125 (2010), S41-S52. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated alpha, delta, epsilon, gamma and mu, respectively. The gamma and alpha classes are further divided into subclasses based on relatively minor differences in the CH sequence and function, e.g., humans express the following subclasses: IgG1, IgG2, IgG3, IgG4, IgA1 and IgA2.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of an antibody for its antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent cellular toxicity.

The term “CDR” as used herein refers to the complementarity determining regions of an antibody variable domain which are hypervariable in sequence and/or form structurally defined loops. Generally, antibodies comprise six CDRs; three in the VH (H1, H2, H3), and three in the VL (L1, L2, L3). In native antibodies, H3 and L3 display the most diversity of the six CDRs, and H3 in particular is believed to play a unique role in conferring fine specificity to antibodies. See, e.g., Xu et al., Immunity 13 (2000) 37-45; Johnson and Wu, Methods Mol. Biol. 248 (2003) 1-25 (Lo, ed., Human Press, Totowa, NJ, 2003). Indeed, naturally occurring camelid antibodies consisting of a heavy chain only are functional and stable in the absence of light chain. See, e.g., Hamers-Casterman et al., Nature 363 (1993) 446-448; Sheriff et al., Nature Struct. Biol. 3 (1996) 733-736. A number of CDR delineations are in use. The ImMunGeneTics (IMGT) unique Lefranc numbering (IMGT numbering) (Lefranc, M.-P. et al., Dev. Comp. Immunol. 27 (2003) 55-77) takes into account sequence conservation, structural data from X-ray diffraction studies, and the characterization of the hypervariable loops in order to define the FR and HVR. The Kabat CDR's are based on sequence variability and are also commonly used (Kabat et al., Sequences of Proteins of Immunological Interest, 5th Ed. Public Health Service, National Institutes of Health, Bethesda, MD. (1991)). Chothia refers instead to the location of the structural loops (Chothia and Lesk, J. Mol. Biol. 196 (1987) 901-917). The CDR delineations used herein are according to the IMGT numbering.

“Framework” or “FR” residues are those variable-domain residues other than the CDR residues as herein defined.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)2 and Fv fragments; diabodies; linear antibodies; single-chain antibody molecules and multi-specific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

A “scFv” (single chain Fv) is a covalently linked VH::VL heterodimer which is usually expressed from a gene fusion including VH and VL encoding genes linked by a peptide-encoding linker. The human scFv fragments of the invention include CDRs that are held in appropriate conformation, for instance by using gene recombination techniques. Divalent and multivalent antibody fragments can form either spontaneously by association of monovalent scFvs, or can be generated by coupling monovalent scFvs by a peptide linker, such as divalent sc(Fv)₂. “dsFv” is a VH::VL heterodimer stabilized by a disulfide bond. “(dsFv)2” denotes two dsFv coupled by a peptide linker.

The term “bi-specific antibody” or “BsAb” denotes an antibody which comprises two different antigen binding sites. Thus, BsAbs are able to bind two different antigens simultaneously. Genetic engineering has been used with increasing frequency to design, modify, and produce antibodies or antibody derivatives with a desired set of binding properties and effector functions as described for instance in EP 2 050 764 A1.

The term “multi-specific antibody” denotes an antibody which comprises two or more different antigen binding sites.

The term “hybridoma” denotes a cell, which is obtained by subjecting a B cell prepared by immunizing a non-human mammal with an antigen to cell fusion with a myeloma cell derived from a mouse or the like which produces a desired monoclonal antibody having an antigen specificity.

The term “diabodies” refers to small antibody fragments prepared by constructing scFv fragments (see preceding paragraph) with short linkers (about 5-10) residues) between the VH and VL domains such that inter-chain but not intra-chain pairing of the V domains is achieved, thereby resulting in a bivalent fragment, i. e., a fragment having two antigen-binding sites. Bispecific diabodies are heterodimers of two “crossover” scFv fragments in which the VH and VL domains of the two antibodies are present on different polypeptide chains. Diabodies are described in greater detail in, for example, EP0404097; WO 93/11161; Hollinger et al., Proc. Natl. Acad. Sci. USA 90 (1993) 6444-6448.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species of belonging to another antibody class or subclass, as well as fragments of such antibodies, so long as they exhibit the desired biological activity.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321 (1986) 522-525; Riechmann et al., Nature 332 (1988) 323-329; and Presta, Curr. Op. Struct. Biol. 2 (1992) 593-596. See also, for example, Vaswani and Hamilton, Ann. Allergy, Asthma and Immunol. 1 (1998) 105-115; Harris, Biochem. Soc. Transactions 23 (1995) 1035-1038; Hurle and Gross, Curr. Op. Biotech. 5 (1994) 428-433; and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227 (1991) 381; Marks et al., J. Mol. Biol., 222 (1991) 581. Also available for the preparation of human monoclonal antibodies are methods described in Dijk and van de Winkel, Curr. Opin. Pharmacol. 5 (2001) 368-74. Human antibodies can be prepared by administering the antigen to a transgenic animal that has been genetically modified to produce partial or full human antibodies in response to antigenic challenge, but whose endogenous loci have been disabled, e.g., OmniAb therapeutic antibody platforms (Ligand Pharmaceuticals), immunized xenomice (see, e.g., U.S. Pat. Nos. 6,075,181 and 6,150,584 regarding Xenomouse technology), etc. See also, for example, Li et al., Proc. Natl. Acad. Set USA 103 (2006) 3557-3562 regarding human antibodies generated via a human B-cell hybridoma technology.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature 256 (1975) 495-497; Hongo et al., Hybridoma 14 (1995) 253-260, Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Sidhu et al., J. Mol. Biol. 338 (2004) 299-310; Lee et al., J. Mol. Biol. 340 (2004) 1073-1093; Fellouse, Proc. Natl. Acad. Sci. USA 101 (2004) 12467-12472; and Lee et al., J. Immunol. Methods 284 (2004) 119-132, and technologies for producing human or human-like antibodies in animals that have parts or all of the human immunoglobulin loci or genes encoding human immunoglobulin sequences (see, e.g., Jakobovits et al., Proc. Natl. Acad. Sci. USA 90 (1993) 2551; Jakobovits et al., Nature 362 (1993) 255-258; Bruggemann et al., Year in Immunol. 7 (1993) 33; Fishwild et al., Nature Biotechnol. 14: (1996) 845-851; Neuberger, Nature Biotechnol. 14 (1996) 826; and Lonberg and Huszar, Intern. Rev. Immunol. 13 (1995) 65-93.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Biotechnology 10 (1992) 779-783 describes affinity maturation by VH- and VL-domain shuffling. Random mutagenesis of HVR and/or framework residues is described by, for example: Barbas et al. Proc Nat. Acad. Sci. USA 91 (1994) 3809-3813; Schier et al. Gene 169 (1995) 147-155; Yelton et al. J. Immunol. 155 (1995) 1994-2004; Jackson et al, J. Immunol. 154 (1995) 3310-9; and Hawkins et al, J. Mol. Biol. 226 (1992) 889-896.

As used herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets.

“Binding affinity” generally refers to the strength of the total sum of non-covalent interactions between a single binding site of a molecule (e.g., of an antibody) and its binding partner (e.g., an antigen). Unless indicated otherwise, as used herein, “binding affinity”, “bind to”, “binds to” or “binding to” refers to intrinsic binding affinity that reflects a 1 to 1 interaction between members of a binding pair (e.g., antibody Fab fragment and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity, i.e. binding strength are described in the following.

The “K_{D}” or “K_{D} value” according to this invention can be measured by a radiolabeled antigen binding assay (RIA) performed with the Fab version of the antibody and antigen molecule, or by using surface-plasmon resonance assays using a BIACORE instrument (BIAcore, Inc., Piscataway, NJ), or by using a biolayer interferometry assay using an Octet instrument (Forté bio, Fremont, CA).

As used herein, the term “conjugate” refers to a chemical (non-biological) therapeutic agent covalently linked to an antibody, as opposed to “complex” which means a chemical (non-biological) therapeutic agent non-covalently bound by the variable regions (CDR's) of an antibody.

By “purified” or “isolated” it is meant, when referring to a polypeptide (e.g., an antibody) or a nucleotide sequence, that the indicated molecule is present in the substantial absence of other biological macromolecules of the same type. The term “purified” as used herein means at least 75%, 85%, 95%, 96%, 97%, or 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

The term “degron” as used herein refers to the degrading moiety of a PROTAC, which is a Von-Hippel-Lindau (VHL) ligand.

The term “warhead” as used herein refers to the moiety of a PROTAC which binds to the to-be-degraded protein (e.g., an inhibitor or such target protein). The warhead moiety is hereinbelow also referred to as “target protein binder” or “protein binder” or “PB”.

### Antibodies and Antibody-PROTAC Complexes (PAX) of the Invention

The inventors have succeeded in generating and selecting specific anti-PROTAC antibodies, in particular anti-VHL-ligand antibodies, wherein the antibodies specifically bind to the VHL ligand degron of PROTAC's.

Hence, in an aspect the invention relates to antibodies, which bind to a VHL ligand such as VH032, or derivatives thereof.

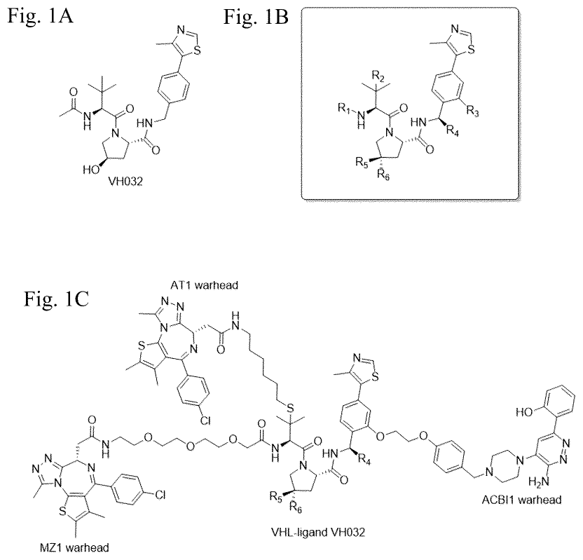

The anti-PROTAC antibodies generated by the inventors are able to bind to the VHL ligand VH032, while tolerating various modifications as outlined by FIG. 1A-C and Table 1 for MIC1- and MIC2-derived antibodies. The antibody tolerates all investigated substitutions in position R₁ and R₂ which include several distinct linker structures that connect VH032 and the target protein binder. In R₃, hydrogen and hydroxyl substitutions are tolerated while antibody binding was suppressed if the target protein binding moiety was connected via a linker to R₃. R₄, can be hydrogen of methyl. R₅ and R₆ can both contain a hydroxyl group given the respective other position is a hydrogen. No substitution was identified in position R₁, R₂, R₅ and R₆ that was not tolerated.

A literature review-based structure analysis revealed that 49.2% of PROTACs engaging VHL are based on the VHL ligand VH032. 29.5% of VHL-based PROTACs utilize a close derivate of VH032 which carries an additional methyl group (R₄=Me, FIG. 1B). The linker for warhead attachment is attached here in position R₁ (FIG. 1B). The remaining VHL-based PROTACs use either a different buildup where the connection to the warhead is implemented by linker attachment to R₃ or carry other modifications like hydroxymethyl in R₄. Taken together, the anti-PROTAC antibodies disclosed in this invention are able to bind to at least 79% of currently publicly known VHL-based PROTACs.

TABLE 1
Binding epitope of the antibodies of the invention.
Possible substitutions

| | |
|---|---|
| R₁ | PB—NH—(CH₂—CH₂—O)ₙ—(CH₂—CH₂—CH₂—O)ₘ—CH₂—(C═O)— |
| | [n = 1, m = 1] |
| | PB—NH—(CH₂—CH₂—O)ₙ—CH₂—(C═O)— [n = 3, 4] |
| | PB—(CH₂—CH₂—O)ₙ—(CH₂)₂—(C═O)— [n = 1] |
| | PB—(CH₂—CH₂—O)ₙ—(CH₂)₂—(C═O)— [n = 2] |
| | PB—NH—(CH₂)ₙ—(C═O)— [n = 10] |
| | PB—(C═O)—(CH₂)ₙ—(C═O)— [n = 6] |
| | PB—(CH₂—CH₂—CH₂—O)ₙ—(CH₂)₂—(C═O)— [n = 2] |
| R₂ | PB—NH—(CH2)ₙ—S— [n = 6] |
| R₃ | H, OH |
| R₄ | H |
| R₅ | OH (if R₆ = H) |
| R₆ | OH (if R₅ = H) |

Therefore, in an embodiment the VH032 derivatives can be described by the formula I

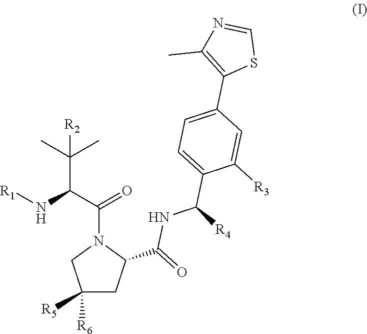

wherein
one of R₁ or R₂ is a linker connected to a warhead (target protein binder, PB), with the proviso that
if R₂ is the warhead-linker, R₁ is acetyl, and
if R₁ is the warhead-linker, R₂ is methyl;
R₃ is H, OH, cyano, F, Cl, amino or methyl;
R₄ is H or methyl;
R₅, R₆ are H or OH, with the proviso that
if R₆ is H, R₅ is OH, and
if R₅ is H, R₆ is OH.

In a more specific embodiment
R₁ is PB-Q-(CH₂—CH₂—O)ₙ—(CH₂—CH₂—CH₂—O)ₘ—(CH₂)ₚ—(C═O)—,
wherein
   PB is a protein binding warhead,
   Q is NH, C═O, or absent,
   n, m are, independently, 0, 1, 2, 3 or 4,
   p is 0-10;
R₂ is methyl;
and R₃, R₄, R₅, and R₆ are as described above.

In even more specific embodiments
R₁ is PB-Q-(CH₂—CH₂—O)ₙ—(CH₂—CH₂—CH₂—O)ₘ—(CH₂)ₚ—(C═O)—,
wherein
   PB is a protein binding warhead;
   Q is NH, C═O, or absent;
   (i) n, m, p are 1; or
   (ii) n is 3 or 4; m is 0, p is 1; or
   (iii) n is 1; m is 0; p is 2; or
   (iv) n is 2; m is 0; p is 2; or
   (v) n, m are 0; p is 6, 7, 8, 9 or 10;
R₂ is methyl;
and R₃, R₄, R₅, and R₆ are as described above.

In very specific embodiments
R₁ is PB—NH—(CH₂—CH₂—O)—(CH₂—CH₂—CH₂—O)ₘ—(CH₂)ₚ—(C═O)—,
wherein
   PB is a protein binding warhead,
   (vi) Q is NH; n, m, p are 1; or
   (vii) Q is NH; n is 3 or 4; m is 0, p is 1; or
   (viii) Q is absent; n is 1; m is 0; p is 2; or
   (ix) Q is absent; n is 2; m is 0; p is 2; or
   (x) Q is NH or C═O; n, m are 0; p is 6, 7, 8, 9 or 10;
R₂ is methyl;
and R₃, R₄, R₅, and R₆ are as described above.

In another more specific embodiment
R₁ is acetyl;
R₂ is PB—NH—(CH₂)ₚ—S—, wherein PB is a protein binding warhead and p is 1, 2, 3, 4, 5, or 6;
and R₃, R₄, R₅, and R₆ are as described above.

In an embodiment (A) the antibody is a full-length antibody whose variable regions comprise CDR's responsible for the PROTAC binding, having the following sequences:

HC CDR1:
(SEQ ID NO: 1)
G Y S X₁ T X₂ X₃ Y;
HC CDR2:
(SEQ ID NO: 2)
I TY S G X₄ T;
HC CDR3:
(SEQ ID NO: 3)
X₅ X₆ Y X₇ X₈ X₉ X₁₀ X₁₁ X₁₂ X₁₃ X₁₄ X₁₆;
LC CDR1:
(SEQ ID NO: 4)
Q X₁₆ X₁₇ X₁₈ X₁₉ X₂₀ X₂₁ X₂₂ X₂₃ X₂₄ Y;
LC CDR2:
(SEQ ID NO: 5)
X₂₅ X₂₆ X₂₇;
LC CDR3:
(SEQ ID NO: 6)
X₂₈ Q X₂₉ X₃₀ X₃₁ X₃₂ P Y T;

wherein: X₁ is I or A; X₂ is G or N; X₃ is D or N; X₄ is G or A; X₅ is A or G; X₆ is K or Y; X₇ is G or Y; X₈ is absent or A; X₉ is absent or V; X₁₀ is absent or P; X₁₁ is D or Y; X₁₂ is G or Y; X₁₃ is G or F; X₁₄ is R or A; X₁₅ is D or H; X₁₆ is S or G; X₁₇ is L or I; X₁₈ is S or absent; X₁₉ is Y or absent; X₂₀ is S or absent; X₂₁ is D or absent, X₂₂ is G or absent; X₂₃ is N or G; X₂₄ is T or N; X₂₅ is L or Y; X₂₆ is V or A; X₂₇ is S or T, X₂₈ is V or L; X₂₉ is S or Y; X₃₀ is I or D; X₃₁ is H or E; and X₃₂ is V or Y.

In a preferred embodiment the CDR sequences are

HC CDR1:
(SEQ ID NO: 7)
G Y S I T G D Y;
HC CDR2:
(SEQ ID NO: 8)
I T Y S G G T;
HC CDR3:
(SEQ ID NO: 9)
A K Y G D G G R D;
LC CDR1:
(SEQ ID NO: 10)
Q S L S Y S D G N T Y;
LC CDR2:
(SEQ ID NO: 11)
L V S;
LC CDR3:
(SEQ ID NO: 12)
V Q S I H V P Y T.

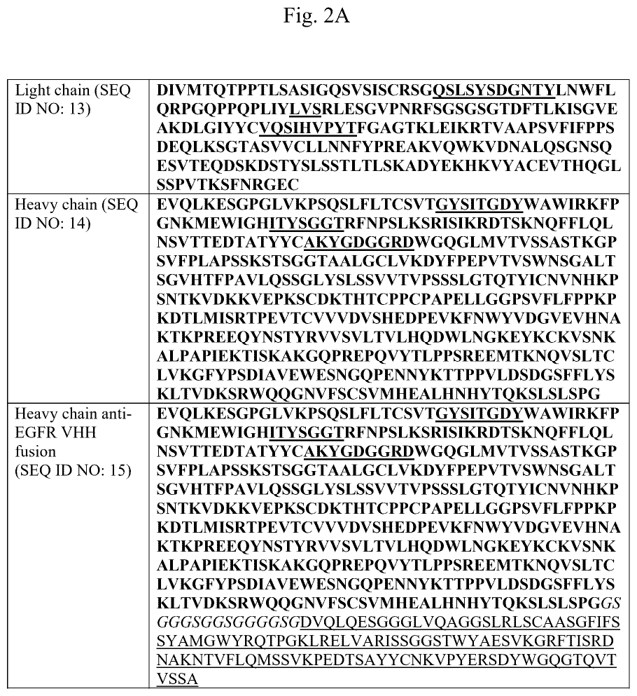

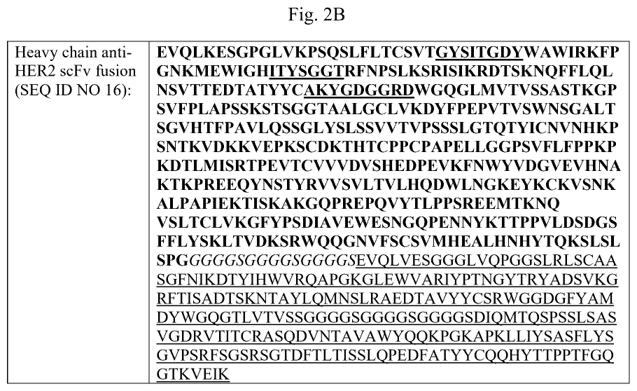

In a very specific embodiment, the antibody corresponds to a pair of light and heavy chain sequences chosen from the MIC 2 sequences SEQ ID Nos: 13 and 14, shown in FIG. 2A.

In another very specific embodiment, the antibody is a BsAb, having the heavy and light chain sequences SEQ ID NO's: 13 and 15, or 13 and 16, as shown in FIG. 2A, to whose heavy chains a HER2 binding VHH, or an EGFR binding scFv, respectively, is fused via a peptide linker, as shown in FIGS. 2A and 2B.

In an embodiment (B) the antibody is a VHH which comprises CDR's responsible for the PROTAC binding having the following sequences:

CDR1:
(SEQ ID NO: 17)
G X₁ X₂ X₃ X₄ X₅ X₆ X₇;
CDR2:
(SEQ ID NO: 18)
X₈ X₉ X₁₀ X₁₁ X₁₂ X₁₃ X₁₄ X₁₅;
CDR3:
(SEQ ID NO: 19)
X₁₅ X₁₆ X₁₇ X₁₈ X₁₉ s X₂₀ X₂₁ X₂ X₂₃ X₂₄
X₂₅ X₂₆ X₂₇ X₂₈ X₂₉ X₃₀ X₃₁ X₃₂ X₃ X₃₄
X₃₅ X₃₆;

wherein: X₁ is F or R; X₂ is T, A, S or R; X₃ is L or F; X₄ is D or N; X₅ is D or T; X₆ is Y or L; X₇ is A or T; X₈ is I, N or L; X₉ is S or T; X₁₀ is S or W; X₁₁ is S or N; X₁₂ is D or G; X₁₃ is G or D; X₁₄ is S or N; X₁₅ is A, or T; X₁₆ is A, S or T; X₁₇ is A, V or I; X₁₈ is S, A, I or D; X₁₉ is T, Y, R or A; X₂₀ is R, Y or G; X₂₁ is V, S, L or T; X₂₂ is L, G, S or C; X₂₃ is S, A, C or P; X₂₄ is T, A, S or N; X₂₅ is P, I, V or D; X₂₆ is absent, V or A; X₂₇ is D, S, R, or absent; X₂₈ is V, G or P; X₂₉ is D, T, G or R; X₃₀ is Q, I, Tor R; X₃₁ is V, K or R; X₃₂ is R, I or Y; X₃₃ is Y, Q, F or A; X₃₄ is V or L; X₃₅ is E, P or D; X₃₆ V, Y or A.
wherein more specifically: X₁ is F; X₂ is T or S; X₃ is L or F; X₄ is D; X₅ is D; X₆ is Y; X₇ is AT; X₈ is I; X₉ is ST; X₁₀ is S; X₁₁ is S; X₁₂ is D; X₁₃ is G; X₁₄ is S; X₁₅ is A, or T; X₁₆ is A or S; X₁₇ is V or A; X₁₈ is A or I; X₁₉ is T or Y; X₂₀ is G or R; X₂₁ is L or S; X₂₂ is C or S; X₂₃ is P or C; X₂₄ is A or S; X₂₅ is V or D; X₂₆ is absent or V; X₂₇ is R, or absent; X₂₈ is G or P; X₂₉ is T or G; X₃₀ is Q, or I; X₃₁ is K or R; X₃₂ is R, I or Y; X₃₃ is F or A; X₃₄ is L; X₃₅ is E, or D; X₃₆ V or Y.

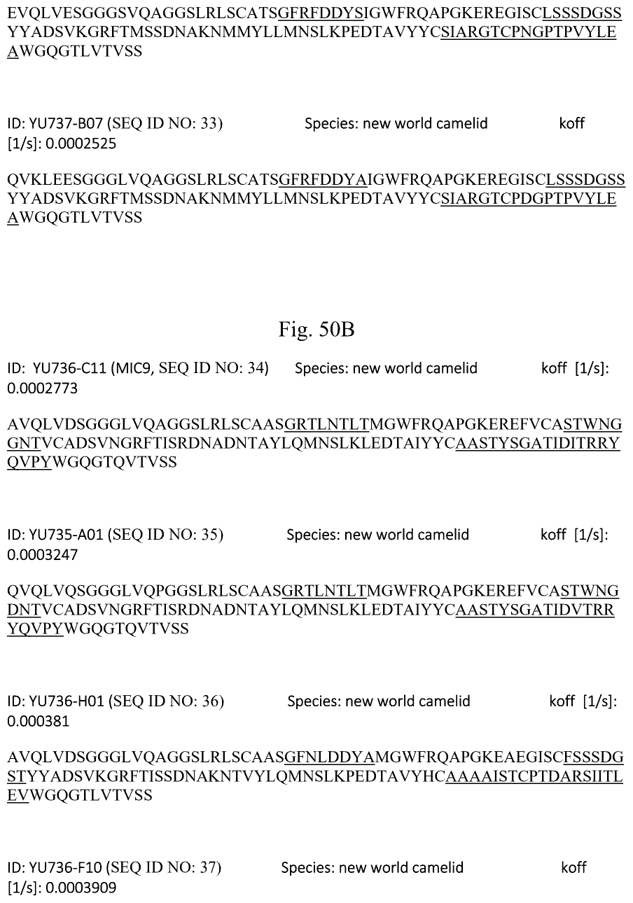

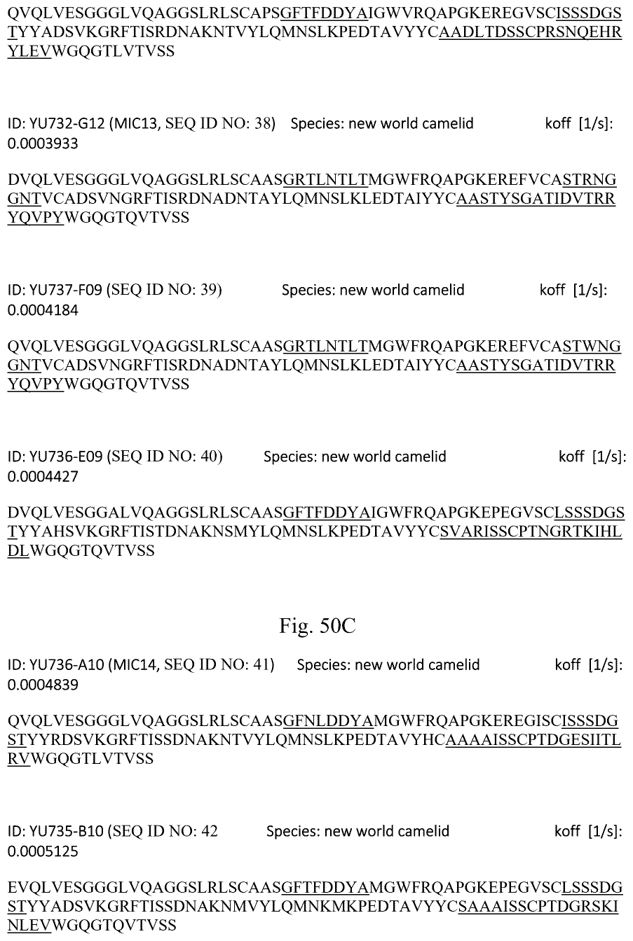

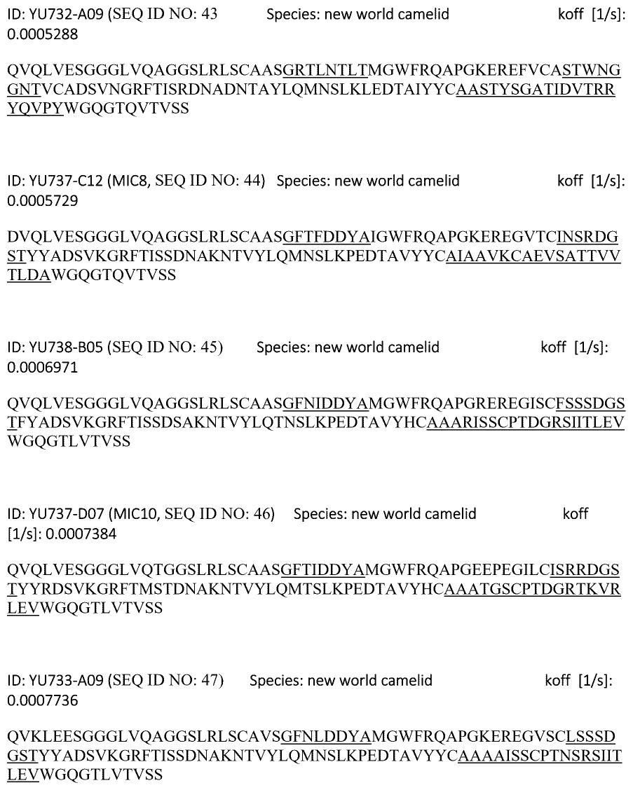

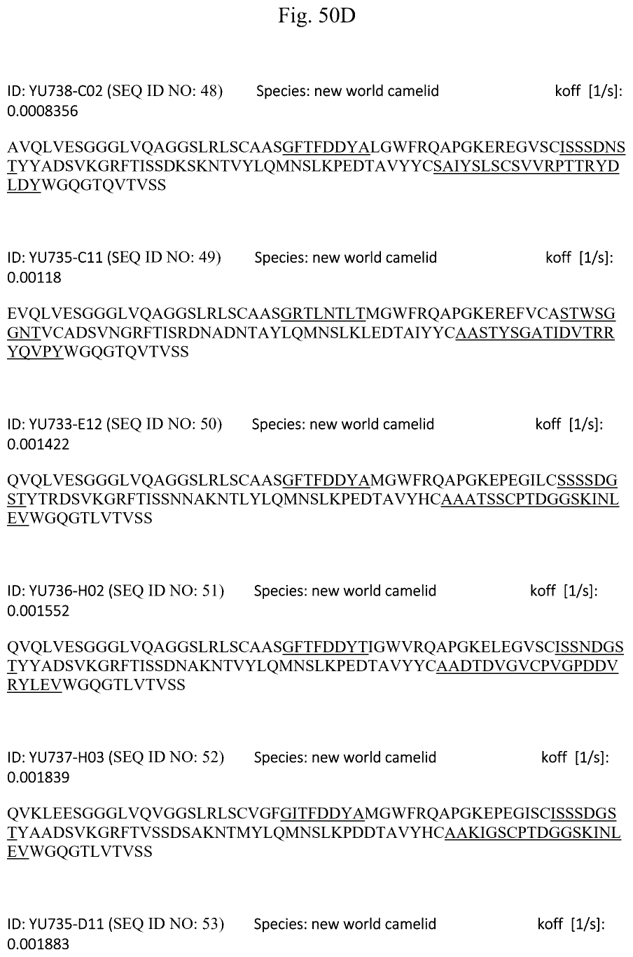

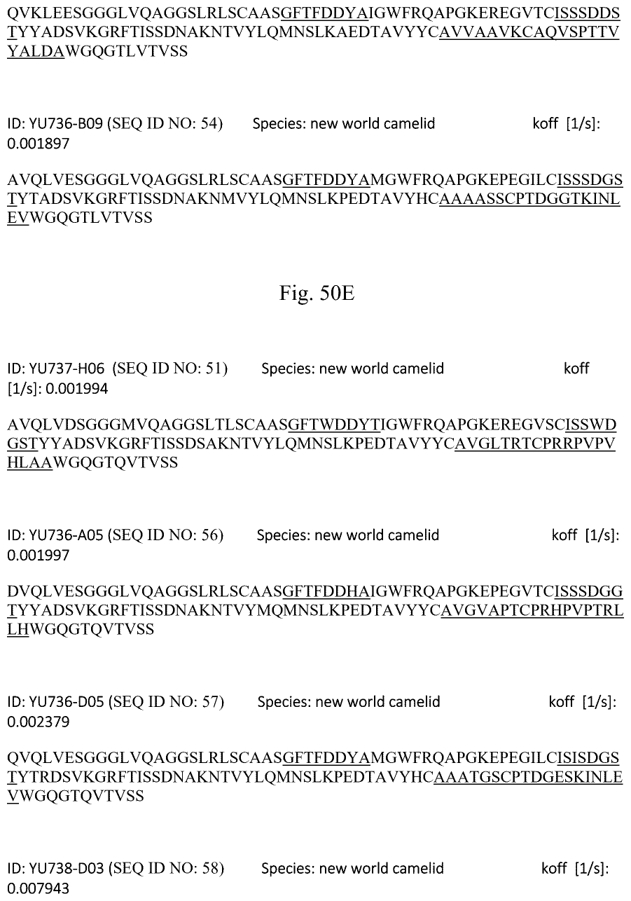

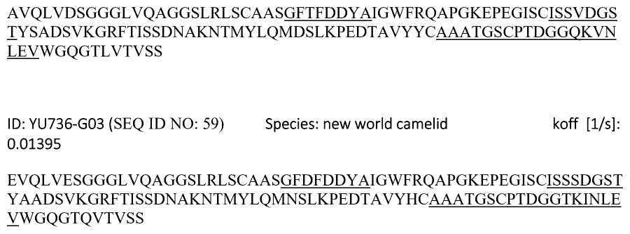

In preferred embodiments the CDR sequences are those of the antibody YU734-F06 (MIC7) shown in FIG. 50A (SEQ ID NO: 20)

CDR1:
(SEQ ID NO: 21)
G F T L D D Y A
CDR2:
(SEQ ID NO: 22)
I S S S D G ST
CDR3:
(SEQ ID NO: 23)
S A I Y R L S C S V V R P T I R Y A L D Y

or those of the antibody YU733-G10 (MIC5) shown in FIG. 50A (SEQ ID NO: 24)

CDR1:
(SEQ ID NO: 25)
G F T F D D Y A
CDR2:
(SEQ ID NO: 26)
I S S S D G S A
CDR3:
(SEQ ID NO: 27)
A V A T G S CP A D G G Q K I F L E V

In a very specific embodiment the VHH corresponds to a sequence chosen from the sequences shown in FIGS. 50A-50E.

In a preferred specific embodiment the VHH corresponds to sequences YU734-F06 (MIC7, SEQ ID NO: 20) or YU733-G10 (MIC5, SEQ ID NO: 24) shown in FIG. 50A:

In an embodiment, the sequences described above for embodiment (B) are part of a BsAb, wherein the N-termini of said sequences are fused, optionally via peptide linkers, to the C-terminus of a full-length antibody capable of binding to a target protein.

In a preferred embodiment, the BsAb comprises peptide linkers. In a more preferred embodiment, the peptide linkers each consist of 1, 2, or 3 repeats of GSGGGSGGSGGGGSG (SEQ ID NO: 28). In an even more preferred embodiment, the peptide linkers each consist of 1 repeat of GSGGGSGGSGGGGSG (SEQ ID NO: 28)

If a full-length antibody, the antibody is preferably of the IgG1 or IgG4 type, to enable FcRn receptor binding.

In an embodiment, the antibody is monospecific and binds only to a PROTAC. The antibody may also be a bi-specific antibody (BsAb), wherein the second specificity is for a target protein.

In the case of a BsAb, the PROTAC binding may be effected through a single chain antibody fused to either the C- or N-terminus, or both termini, of either the heavy or the light chain, or both chains, of a full length antibody, while the target protein binding is effected through the six CDR's of the variable regions of the full length antibody.

Alternatively, the target protein binding is effected through a single chain antibody fused to either the C- or N-terminus, or both termini, of either the heavy or the light chain, or both chains, of a full length antibody, and the PROTAC binding is effected through the six CDR's of the variable regions of the full length antibody.

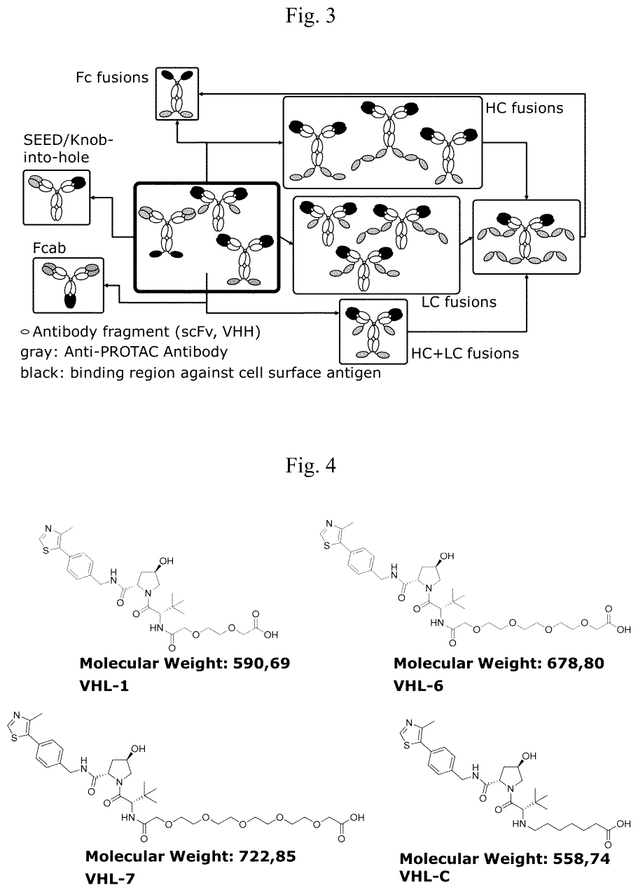

Examples of BsAb variants according to the invention are shown in FIG. 3.

In a preferred embodiment, the target protein of the BsAb according to the invention is a cell surface protein, e.g., a tumor antigen, such as HER2 or EGFR.

### Nucleic Acids, Vectors and Host Cells

Another aspect of the invention relates to an isolated nucleic acid comprising or consisting of a nucleic acid sequence encoding an antibody of the invention as defined above.

Typically, said nucleic acid is a DNA or RNA molecule, which may be included in any suitable vector, such as a plasmid, cosmid, episome, artificial chromosome, phage or a viral vector. The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. Accordingly, a further aspect of the invention relates to a vector comprising a nucleic acid of the invention as defined above. Such vectors may comprise regulatory elements, such as a promoter, enhancer, terminator and the like, to cause or direct expression of said polypeptide upon administration to a subject.

A further aspect of the present invention relates to a host cell which has been transfected, infected or transformed by a nucleic acid and/or a vector according to the invention.

The term “transformation” means the introduction of a “foreign” (i.e. extrinsic) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

The nucleic acids of the invention may be used to produce an antibody of the invention in a suitable expression system. The term “expression system” means a host cell and compatible vector under suitable conditions, e.g., for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, HEK cells, 3T3 cells, COS cells, etc.).

### Methods of Producing Antibodies of the Invention

Antibodies of the invention may be produced by any technique known in the art, such as, without limitation, any chemical, biological, genetic or enzymatic technique, either alone or in combination.

Knowing the amino acid sequence of a desired antibody, one skilled in the art can readily produce said antibodies or immunoglobulin chains using standard techniques for production of polypeptides. For instance, they can be synthesized using well-known solid phase methods using a commercially available peptide synthesis apparatus (such as that made by Applied Biosystems, Foster City, California) and following the manufacturer's instructions. Alternatively, antibodies and immunoglobulin chains of the invention can be produced by recombinant DNA techniques, as is well-known in the art. For example, these polypeptides (e.g., antibodies) can be obtained as DNA expression products after incorporation of DNA sequences encoding the desired polypeptide into expression vectors and introduction of such vectors into suitable eukaryotic or prokaryotic hosts that will express the desired polypeptide, from which they can be later isolated using well-known techniques.

In a further aspect, the invention relates to a method of producing an antibody of the invention, which method comprises the steps consisting of: (I) culturing a transformed host cell according to the invention; (ii) expressing the antibody; and (iii) recovering the expressed antibody. Antibodies of the invention can be suitably separated from the culture medium by conventional immunoglobulin purification procedures such as, for example, protein A-Sepharose, hydroxyapatite chromatography, gel electrophoresis, dialysis, or affinity chromatography. A Fab of the present invention can be obtained by treating an antibody of the invention (e.g., an IgG) with a protease, such as papaine. Also, the Fab can be produced by inserting DNA sequences encoding both chains of the Fab of the antibody into a vector for prokaryotic expression, or for eukaryotic expression, and introducing the vector into prokaryotic or eukaryotic cells (as appropriate) to express the Fab.

A F(ab′)2 of the present invention can be obtained treating an antibody of the invention (e.g., an IgG) with a protease, pepsin. Also, the F(ab′)2 can be produced by binding a Fab′ described below via a thioether bond or a disulfide bond.

A Fab′ of the present invention can be obtained by treating F(ab′)2 of the invention with a reducing agent, such as dithiothreitol. Also, the Fab′ can be produced by inserting DNA sequences encoding Fab′ chains of the antibody into a vector for prokaryotic expression, or a vector for eukaryotic expression, and introducing the vector into prokaryotic or eukaryotic cells (as appropriate) to perform its expression.

### Solubilizing and Stabilizing PROTACs

PROTACs are often hydrophobic, which limits their in vivo applicability, while antibodies are generally sufficiently soluble. Therefore, the binding of the anti-PROTAC antibodies of the invention to the degron part of the PROTAC, partly masks the PROTAC from the surrounding solvent. The net result of this is a solubilizing effect of the antibody binding, i.e. an improved solubility, which is advantageous for in vivo administration and xenograft studies.

Moreover, PROTACs have several metabolic soft spots in the warhead, linker and degron part (Goracci, L. et al., J. Med. Chem. 63 (2020) 11615-11638) which limits their metabolic stability.

By complexation of PROTACs with an antibody of the invention, the steric accessibility of the PROTAC to metabolic enzymes is limited, leading to improved metabolic stability.

### Pharmaceutical Compositions

The PAX of the invention may be combined with pharmaceutically acceptable carriers, diluents and/or excipients, and optionally with sustained-release matrices including but not limited to the classes of biodegradable polymers, non-biodegradable polymers, lipids or sugars, to form pharmaceutical compositions.

Thus, another aspect of the invention relates to a pharmaceutical composition comprising PAX of the invention and a pharmaceutically acceptable carrier, diluent and/or excipient.

“Pharmaceutical” or “pharmaceutically acceptable” refers to molecular entities and compositions that do not produce an adverse, allergic or other unwanted reaction when administered to a mammal, especially a human, as appropriate. A pharmaceutically acceptable carrier, diluent or excipient refers to a non-toxic solid, semi-solid or liquid filler, diluent, encapsulating material or formulation auxiliary of any type.

As used herein, “pharmaceutically acceptable carriers” include any and all solvents, dispersion media, coatings, antibacterial and antifungal agents, and the like that are physiologically compatible. Examples of suitable carriers, diluents and/or excipients include, but are not limited to, one or more of water, amino acids, saline, phosphate buffered saline, buffer phosphate, acetate, citrate, succinate; amino acids and derivates such as histidine, arginine, glycine, proline, glycylglycine; inorganic salts such as sodium or calcium chloride; sugars or polyalcohols such as dextrose, glycerol, ethanol, sucrose, trehalose, mannitol; surfactants such as polysorbate 80, polysorbate 20, poloxamer 188; and the like, as well as combinations thereof. In many cases, it will be useful to include isotonic agents, such as sugars, polyalcohols, or sodium chloride in a pharmaceutical composition, and the formulation may also contain an antioxidant such as tryptamine and/or a stabilizing agent such as Tween 20.

The form of the pharmaceutical compositions, the route of administration, the dosage and the regimen naturally depend upon the condition to be treated, the severity of the illness, the age, weight, and gender of the patient, etc.

The pharmaceutical compositions of the invention can be formulated for parenteral, intravenous, intramuscular, or subcutaneous administration and the like.

In an embodiment, the pharmaceutical compositions contain vehicles which are pharmaceutically acceptable for a formulation for injection. These may be isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical composition can be administered through drug combination devices. The doses used for the administration can be adapted as a function of various parameters, and for instance as a function of the mode of administration used, of the relevant pathology, or alternatively of the desired duration of treatment.

To prepare pharmaceutical compositions, an effective amount of the PAX of the invention may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions; in all such cases, the form must be sterile and injectable with the appropriate device or system for delivery without degradation, and it must be stable under the conditions of manufacture and storage and must be preserved against the contaminating action of microorganisms, such as bacteria and fungi.

Sterile injectable solutions can be prepared by incorporating the active compounds in the required amount in the appropriate solvent with any of the other ingredients enumerated above, as required, followed by sterile filtration. Generally, dispersions can be prepared by incorporating the various sterilized active ingredients into a sterile vehicle which contains the basic dispersion medium and the required other ingredients from those enumerated above. In the case of sterile powders for the preparation of sterile injectable solutions, methods of preparation include vacuum-drying and freeze-drying techniques which yield a powder of the active ingredient plus any additional desired ingredient from a previously sterile-filtered solution thereof.

For parenteral administration in an aqueous solution, for example, the solution can be suitably buffered if necessary and the liquid diluent first rendered Isotonic with sufficient saline or glucose. These aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The PAX of the invention may be formulated within a therapeutic mixture to comprise, e.g., about 0.01 to 100 milligrams per dose or the like.

In a specific aspect, a first pharmaceutical composition comprises the PAX, and a second pharmaceutical composition comprises only the PROTAC component of the said PAX.

In another specific aspect, a first pharmaceutical composition comprises only the antibody component of the PAX, and a second pharmaceutical composition comprises only the PROTAC component of the said PAX.

### Therapeutic Methods and Uses

As described above and below, the inventors have found that the PAX of the invention are able to effectively deliver a given PROTAC to a target cell. Furthermore, they have shown that the said PAX release their PROTAC payloads into the cytosol of a target cell, where the PROTACs mediate the degradation of their target proteins.

Accordingly, in an embodiment, the present invention provides the PAX, or pharmaceutical composition thereof, for use as a medicament.

In another aspect, the invention provides methods of treating diseases which benefit from the degradation of the PROTAC's target proteins, e.g., cancer, comprising administering the PAX or pharmaceutical composition of the invention, to a subject in need thereof.

In a specific embodiment, the PAX, or pharmaceutical composition comprising said PAX, is administered first, followed by a subsequent administration of the PROTAC component of the PAX alone, or pharmaceutical composition comprising said PROTAC, allowing antibodies having released their PROTAC payloads, to bind further PROTAC components, and deliver those to the target cells.

In another specific embodiment, the antibody component of the PAX, or pharmaceutical composition comprising said antibody, is administered first, and the PROTAC component of the PAX, or pharmaceutical composition comprising said PROTAC, is administered subsequently, allowing antibodies to bind their PROTAC “antigens” in vivo, and deliver those to the target cells.

In further aspects, the antibody component of the PAX is used (i) to increase the in vivo half-life of the PROTAC (I.e., to slow down degradation), (ii) as an extended release formulation of the PROTAC (i.e. allowing it to be effective over a longer period of time), or (iii) as an antidote to counteract toxic effects of PROTACs, by temporarily neutralizing them, so that the toxicity threshold is undercut.

In an embodiment, the antibody component of the PAX is an antibody fragment, such as an Fc fragment.

### Non-Therapeutic Uses

The anti-PROTAC antibodies of the invention, preferably mono-specific antibodies, may also be used for non-therapeutic applications, such as detecting, quantifying or purifying PROTACs.

In an embodiment, for such uses, the antibodies are immobilized on a chromatographic column or some other solid support.

### Kits

Finally, the invention also provides kits comprising at least one antibody or PAX of the invention.

Kits containing PAX of the invention can be used for therapeutic purposes, which may be monotherapies, or combination therapies, in which case they contain one or more further pharmaceutical compositions, comprising additional pharmaceutical ingredients. The therapeutic kits may also contain a package insert with administration instructions.

Kits containing antibodies of the invention may also be used for diagnostic or detection purposes. In such kits the antibody typically is coupled to a solid support, e.g., a tissue culture plate or beads (e.g., sepharose beads), and is used to detect and/or quantify a PROTAC in vitro, e.g., in an ELISA or a Western blot. Such an antibody useful for detection may be provided with a label such as a fluorescent or radiolabel.

### EXAMPLES

### Anti-PROTAC Antibodies MIC1 and MIC2

### Hapten Conjugation

For the preparation of immunogens and screening compounds, VH032-based haptens (VHL-1, VHL-6, VHL-7, VHL-c (FIG. 4) were dissolved separately in conjugation buffer (0.1 M MES, 0.9 M NaCl, 0.02% sodium azide; pH 4.7) to a final concentration of 4 mg/mL and mixed either with a solution of 10 mg/ml Bovine Serum Albumin (BSA) or 10 mg/mL keyhole limpet hemocyanine (KLH), 10 mg/ml cationic BSA (cBSA) and 7 mg/mL human Fc (huFc) (final protein: hapten molar ratio of 1:100).

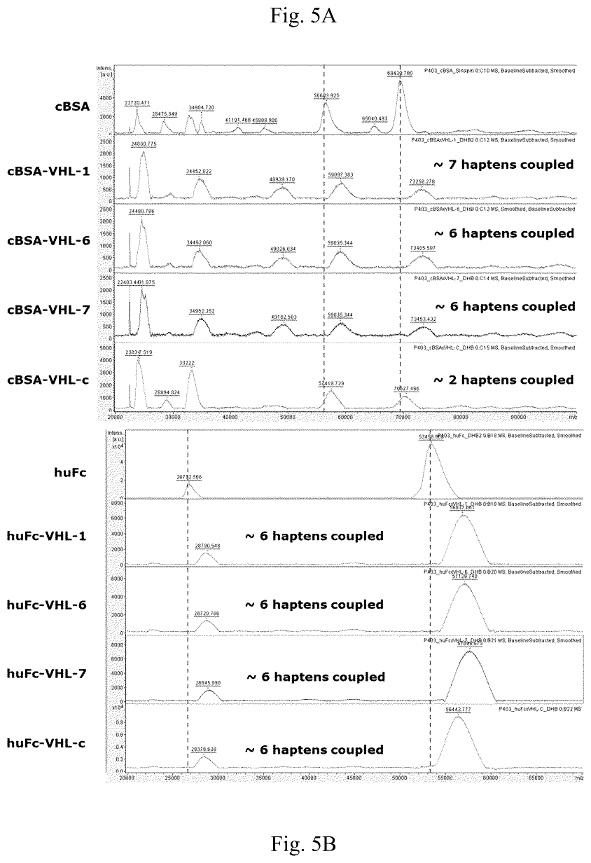

To this mixture a 10 mg/mL aqueous solution of 1-ethyl-3-(3-dimethylaminopropyl) carbodiimide (EDC) was added (final protein EDC molar ratio 1:1750) and the reaction was incubated overnight. Reaction mixes were purified with Zeba Spin Desalting Columns pre-equilibrated in phosphate buffered saline (PBS, 0.137 M NaCl, 0.0027 M KCl, 0.01 M Na₂HPO₄, 0.0018 M KH₂PO₄, pH 7.4) (7K MWCO, Thermo Scientific). Protein concentration was determined with Bradford reagent using unconjugated KLH or BSA as standard. Conjugation efficiency was tested for BSA and huFc conjugates by MALDI-MS. An approximate hapten-to-carrier protein ratio could be derived for each individual conjugation (FIG. 5A-5B).

### Immunization and Hybridoma Screening

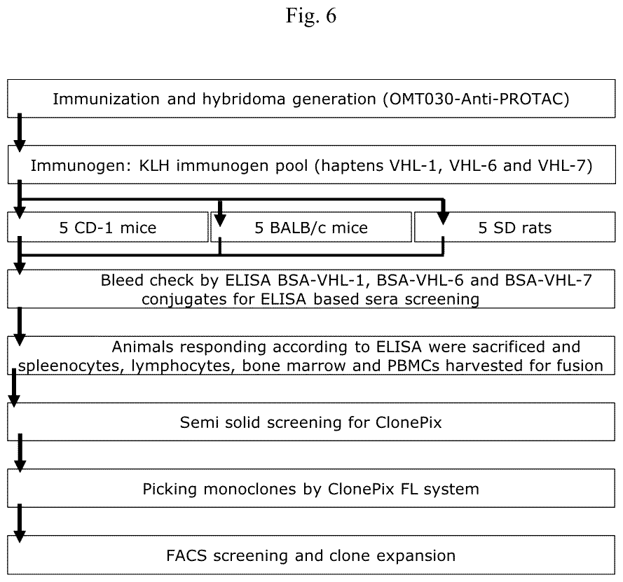

BALB/c and CD-1 mice as well as SD rats were immunized with an equimolar mixture of haptens VHL-1, VHL-6 and VHL-7 (FIG. 4) conjugated to Keyhole Limpet Hemocyanin (KLH) as immunogenic carrier protein. After injection of the immunogens, the serum of all animals was checked for antibody response using ELISA with BSA-hapten conjugates trice. The animals showed immune response leading to immune repertoire screening for VHL-ligand binding antibodies using hybridoma screening. FIG. 6 shows the detailed workplan.

### Manufacturing of the MIC Antibodies

Monospecific antibodies were expressed by transient transfection of heavy and light chains in Expi293F cells following the manufacturer's instructions using the corresponding transfection kit and media from Life Technologies. 50 μg plasmid DNA for heavy and 100 μg plasmid DNA for light chain were diluted in 10 mL OptiMEM medium. 536 μL ExpiFectamine was added to 10 mL OptiMEM followed by incubation for 5 min at room temperature. Then, the plasmid dilution was added. After 20 min of incubation at room temperature the mixture was added to 180 mL Expi293 cells at a cell density of 2.9×10⁶ viable cells per mL. The cell suspension was incubated at 37° C., 5% CO₂, 80 rpm in a humid atmosphere. After 18-22 h, 1 mL Enhancer 1 and 10 mL Enhancer 2 were added. After additional incubation for 4 days at 37° C. with 5% CO₂ while shaking in a humid atmosphere, the antibody was harvested by centrifugation (30 min at 3000 rpm) and sterile filtered with 0.22 μm bottle-top filter.

The supernatant was purified by protein A affinity chromatography using an ÄktaXpress system followed by preparative SEC (HiLoad 16/60 Superdex 200 prep grade) to remove aggregates. Antibodies were concentrated using Ultra centrifugal filter units (30 k MWCO, Amicon), sterile filtered and protein concentration was determined by UV-VIS spectroscopy at 280 nm. The antibodies were characterized by analytical SEC, SDS-PAGE and LC-MS regarding identity.

### Versatility of Antibody Binding to PROTACs

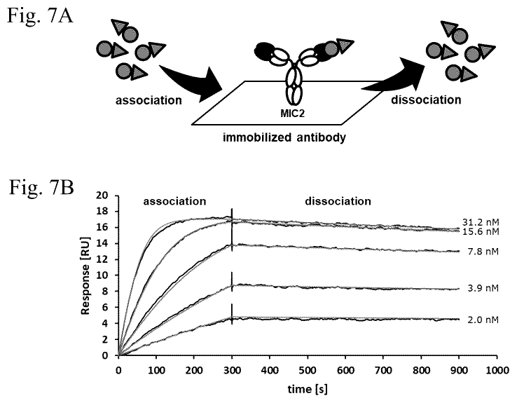

The binding affinities of hit antibodies MIC1 and MIC2 to a diverse set of VH032-based PROTACs (FIGS. 8A-8B) was determined by surface plasmon resonance (SPR) in a Biacore T200 instrument. The running buffer consist of PBS, 0.05% Tween-20, 2% DMSO and temperature and flow rate were set to 30° C. and 30 μL/min, respectively. The assay setup is depicted exemplarily in FIG. 7A. CM5 sensor chips were coated with antibody (=ligand; 2500 RU) using standard EDC/NHS chemistry. PROTACs (=analytes) were serially diluted (1 μM-1.9 nM) in running buffer, injected to the instrument in consecutive runs and were captured by the antibody resulting in an increase of the corresponding SPR response. After the association step (300 s), running buffer was injected for 600 s and the PROTAC dissociated from the antibody leading to signal decrease. After each run, residual bound PROTAC was removed from the immobilized antibody by injection of 10 mM glycine/HCl, pH 1.5 for 2×30 s regenerating the antibody for the next association and dissociation cycle. To compensate for matrix effects, the measured SPR response signal was subtracted by the analyte response to an inactivated (EDC/NHS, ethanol amine) reference surface omitting the ligand. Furthermore, a DMSO solvent correction was performed and analyte response was subtracted by the running buffer signal. The corrected response was fitted by a 1:1 kinetic binding model yielding the on- (kₒₙ) and off-rate (k_{off}) of the PROTAC, as well as its dissociation constant (K_{D}).

The affinity parameters are summarized in tabular form (Table 2) for MIC2 combined with 16 distinct PROTACs with corresponding K_{D}, association rate kₒₙ and dissociation rate k_{off}. 13 of 16 (81.3%) PROTACs were bound by MIC2 with nanomolar K_{D}. Table 2 contains also affinity data for MIC1 which was able to bind to 1 PROTAC that was not bound by MIC2.

TABLE 2
Overview on affinity parameters K_{D}, association rate
kₒₙ, dissociation rat k_{off} for combinations of MIC2 and
PROTACs (see structures, FIG. 8A-8B obtained using a 1:1 kinetic
binding model for MIC2 and K_{D} for binding of PROTACs
to MIC1 derived from a steady-state model.

| Antibody | PROTAC | K_{D} [nM] | kₒₙ [1/Ms] | k_{off} [1/s] |
|---|---|---|---|---|
| MIC1/MIC2 | MZ1 | 4.9/0.12 | NM/1.10E+06 | NM/1.30E−04 |
| MIC1/MIC2 | MZP54 | NM/0.42 | NM/2.00E+05 | NM/8.40E−05 |
| MIC1/MIC2 | MZP55 | NM/0.63 | NM/1.20E+05 | NM/7.40E−05 |
| MIC1/MIC2 | dTRIM24 | NM/0.42 | NM/3.92E+05 | NM/1.64E−04 |
| MIC1/MIC2 | FAKd1 | NM/0.31 | NM/1.71E+05 | NM/5.37E−05 |
| MIC1/MIC2 | SIAIS178 | NM/0.41 | NM/1.05E+05 | NM/4.25E−05 |
| MIC1/MIC2 | BETTY2 | 6.7/0.11 | NM/7.37E+05 | NM/7.73E−05 |
| MIC1/MIC2 | GNE987 | NM/1.9 | NM/2.20E+04 | NM/4.10E−05 |
| MIC1/MIC2 | cMETd1 | NM/3.6 | NM/2.60E+04 | NM/9.30E−05 |
| MIC1/MIC2 | cMETd1 OH | NM/19.1 | NM/9.33E+03 | NM/1.78E−04 |
| | diastereomer | | | |
| MIC1/MIC2 | AT1 | NM/6 | NM/3.10E+05 | NM/1.90E−03 |
| MIC1/MIC2 | FLT3d1 | NM/5.55 | NM/1.63E+04 | NM/9.05E−05 |
| MIC1/MIC2 | BETTY3 | NM/1.79 | NM/3.76E+05 | NM/6.75E−04 |
| MIC1/MIC2 | VZ185 | NM/NB | NM/NB | NM/NB |
| MIC1/MIC2 | ARV771 | 6.5/NB | NM/NB | NM/NB |
| MIC1/MIC2 | ACBI1 | NM/NB | NM/NB | NM/NB |

NM = Not measured; NB = No binding.

### Manufacturing of PAX Using MIC2 and PROTAC

The antibody MIC2 was reformatted into a bispecific format by genetically fusing a glycine-serine linker sequence followed by an anti-EGFR VHH antibody sequence or an anti-HER2 scFv sequence C-terminally to the heavy chains of MIC2. The production took place as described before for the monospecific antibodies. EGFR and HER2 were chosen since they are tumor-associated antigens expressed on the cell surface of cells and hence are accessible to antibody binding. Furthermore, they've been applied in development of antibody-drug conjugates already which underpins their utility as targets with regard to availability of tumor models, sufficient expression and internalization.

Complexation was performed as follows: 10 μM (final) aEGFRxMIC2 or aHER2xMIC2 were mixed in PBS pH 7.4 with GNE987 in DMSO to a achieve the desired loading (Table 3). 5% Tween-20 in PBS pH 7.4 solution was added to a final concentration of 0.3%. The samples were incubated on a ThermoMixer for 3 h at 25° C. while shaking at 650 rpm. No aqueous Tween-20 was added for complex investigation using size-exclusion chromatography.

TABLE 3
Overview on required final PROTAC concentrations
to achieve desired loading.

| | FINAL PROTAC |
|---|---|
| LOADING | CONCENTRATION [μM] |
| 0% | 0 |
| 10% | 2 |
| 25% | 5 |
| 50% | 10 |
| 75% | 15 |
| 100% | 20 |
| 200% | 40 |

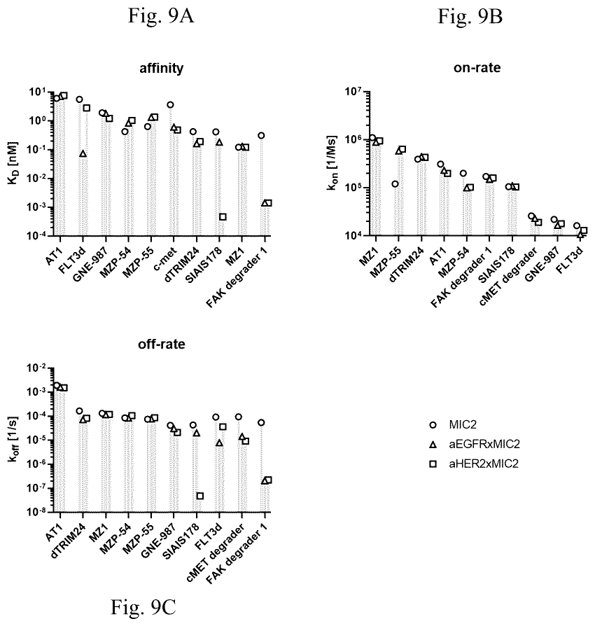

The affinity towards a set of PROTACs was assessed again to confirm PROTAC binding. Overall, the affinity of the bispecific antibodies was comparable to the affinity of MIC2 (FIGS. 9A-9C).

### Loading-Dependent Complexation

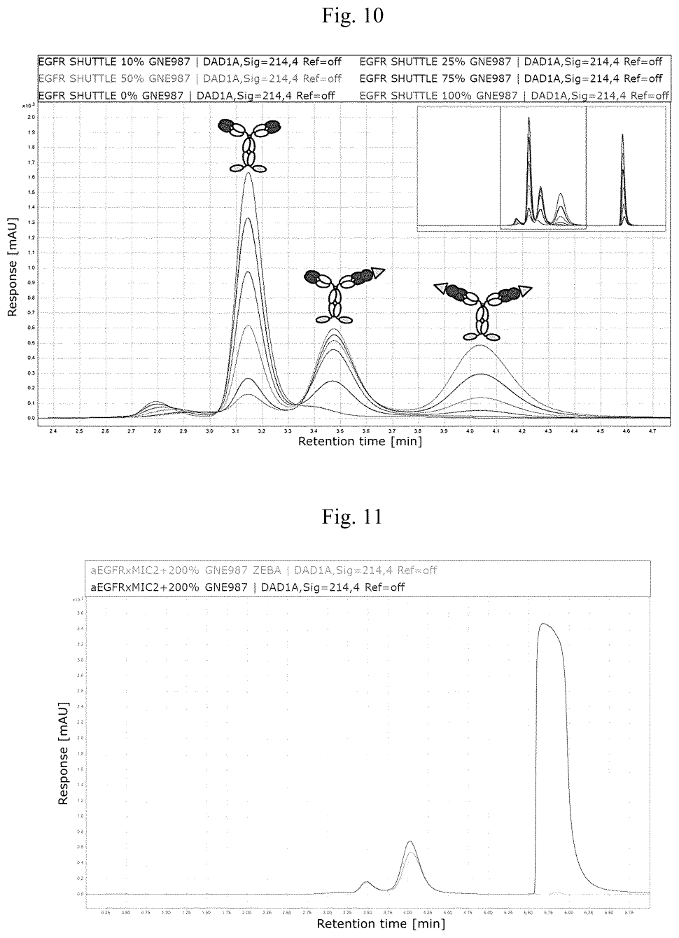

aEGFRxMIC2 was loaded with 0, 10, 25, 50, 75 and 100% GNE987 PROTAC and injected immediately into the SEC system. The antibody aEGFRxMIC2 elutes at 3.15 min. A second peak appears at 3.48 min with increased loading corresponding to the antibody aEGFRxMIC2 loaded with one PROTAC molecule. Further increasing the loading leads to the appearance of a third peak at 4.04 min (two PROTACs per antibody). Overall, the peak distribution is shifted toward the later elution times with increasing loading (FIG. 10).

### Purification of Fully Loaded Complexes and Complex Stability

The aEGFRxMIC2 antibody was loaded with 200% GNE987. The sample was split in half and one portion was desalted into PBS pH 7.4 using Zeba Spin Desalting Columns, 40K MWCO, 75 μL according to the manufacturers' instructions. The chromatogram shows the removal of DMSO and PROTAC eluting at around 5.7 min (FIG. 11).

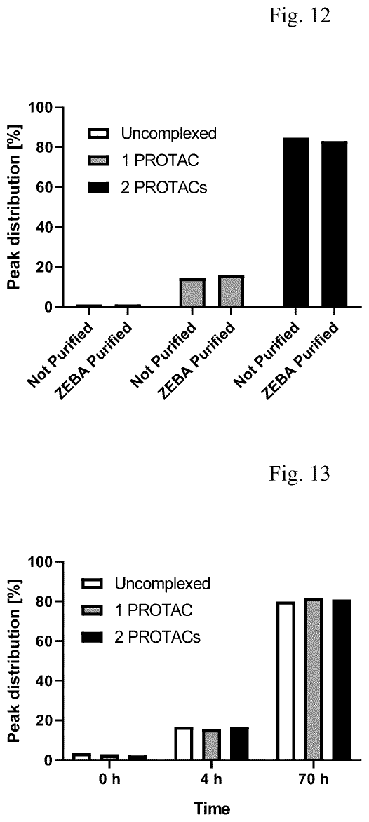

Peaks from FIG. 11 were integrated and the peak distribution was plotted (FIG. 12) for the unpurified and purified antibody-PROTAC complex. The peak distribution remains unaffected by the desalting process indicating that a PAX can be purified from excess of PROTAC or other small molecules without any impact on PROTAC loading.

The aEGFRxMIC2+GNE987 complex was studied at a protein concentration of 10 μM over the course of 70 h at room temperature to assess complex stability over time in PBS pH 7.4. The peak distribution is unaffected by increased incubation time (FIG. 13) indicating complex stability over 70 h.

### Functionality of the PAX Approach

### Covalent PROTAC-ADCs

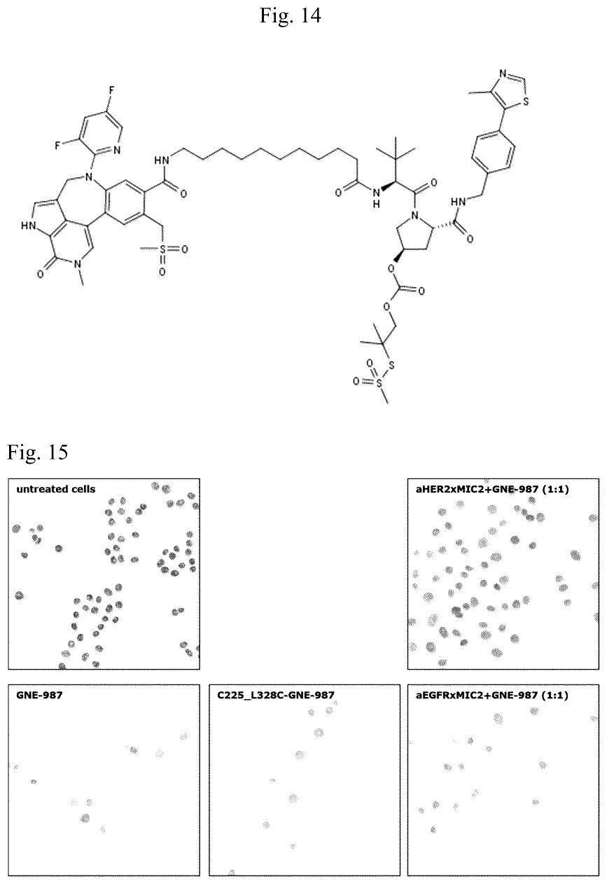

Control PROTAC-ADC was prepared according to WO 2020086858 A1 by conjugation of 1 (FIG. 14) to the anti-EGFR antibody cetuximab (C225) which carried a L328C mutation. The final conjugate had a drug-to-antibody ratio of 1.62 according to mass spec while having a monomeric content of 97.0%.

### BRD4 Degradation

BRD4 was chosen as the model protein for the disclosed invention due to the strong pharmacological effect (cell death) that is induced upon degradation of BRD4. For the evaluation of targeted BRD4 degradation, MDA-MB-468 cells were seeded into a black 96 well clear bottom plates (10,000 cells/well) followed by incubation (37° C., 5% CO2) in a humid chamber overnight. Test compounds were added using a D300e digital dispenser (Tecan) and incubated for 43 h (37° C., 5% CO2, humid chamber). Cells were washed 3× with PBS, fixed in 2% (v/V) formaldehyde for 15 min at rt and washed again (3×). To permeabilize cells, 0.2% (v/V) Triton-X-100 was added for 10 min at rt and removed by washing with PBS (3×). Wells were blocked with 3% (w/v) BSA in PBS for 60 min at rt, washed trice, and cells were incubated with 2.3 μg/mL rabbit anti-BRD4 antibody (Abcam) diluted in 3% BSA/PBS for 60 min at 4° C. overnight. After washing with PBS (3×), cells were incubated with a secondary labeling AF488 goat anti-rabbit antibody (5 μg/mL in 3% BSA/PBS) for 120 min at rt in the dark, washed trice, and nuclei were stained with Hoechst 33342 (5 μg/mL 3% BSA/PBS) for 90 min at rt in the dark. After a final washing step, cells were preserved in 0.1% (w/V) sodium azide and transferred to a Cytation 5 cell imaging reader (Biotek). Images were taken applying the DAPI (nucleus) and green fluorescent protein (BRD4) filter cubes and processed with the BioTek gen5 data analysis software. For quantitative analysis of nuclear BRD4 levels, only the green fluorescence (AF488) co-localized with DAPI staining was counted. Green fluorescence was normalized to cell number and expressed relative to untreated cells.

FIG. 15 shows exemplary images of the BRD4 levels. It is important to note that a higher fluorescence indicates a higher availability of BRD4 in MDAMB468 cells. Untreated cells show the strongest green fluorescence which can be suppressed by BRD4 degradation mediated by GNE987, the anti-EGFR PROTAC-Antibody-drug conjugate C225-L328C-GNE987 based on the EGFR antibody Cetuximab (short C225) and aEGFRxMIC2 loaded with 50% GNE987. Those molecules have comparable effects on BRD4 degradation. The induction of degradation by GNE987 can be reduced by the complexation with aHER2xMIC2 which does not bind to MDAMB468 cells.

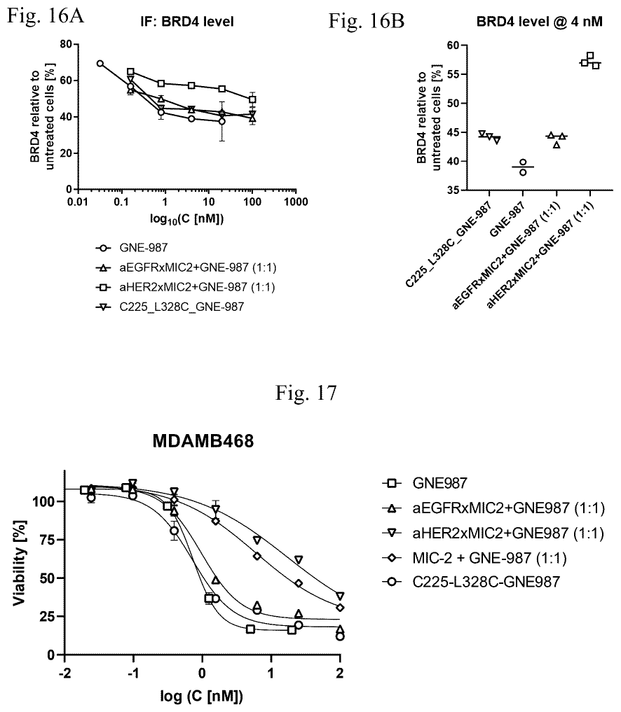

The fluorescence in the nucleus can be used to quantify the degradation effects of the analytes (FIGS. 16A-16B). The trend already observed in the fluorescence images becomes again visible over the whole range of concentrations (FIG. 16A). A zoom on BRD4 values at 4 nM treatment concentration was made to facilitate simpler comparison (FIG. 16 B). GNE987 had the strongest degradation effects reaching down to a 39.0% of remaining BRD4. aEGFRxMIC2 loaded with GNE987 and C225-L328C-GNE987 degraded BRD4 in a nearly identical manner (44.0% and 44.2%, respectively). The degradation induced by aHER2xMIC2 loaded with GNE987 amounts to 57.3% which was 13.3% lower compared to the corresponding aEGFRxMIC2+GNE987 complex.

### Conclusion

aEGFRxMIC2 complexed with GNE987 induced BRD4 degradation in EGFR-expressing MDAMB468 cells similar to PROTAC GNE987 and PROTAC-antibody-drug conjugate C225-L328C-GNE987. Decreased BRD4 degradation was observed for aHER2xMIC2 complexed with GNE987 since aHER2xMIC2+GNE987 complexes cannot enter MDAMB468 cells due to lack of HER2 expression.

### Selective Cell Killing Through Antibody-Mediated Delivery of BRD4-Degrading PROTACs

BRD4 degradation has been shown to induce potent cell killing on several cell lines with potencies in the nano- to subnanomolar range (Pillow, T. H. et al., ChemMedChem 15 (2020) 17-25).

2000 cells per well were seeded into white-opaque 384-well plates followed by overnight incubation in a humid chamber at 37° C. and 5% CO₂. Complexation was performed as described in chapter 7.4.

The solutions were added to the cells based on the antibody concentration using Tecan D300e dispenser and all wells were normalized to the same volume using 0.3% TW20 in PBS pH 7.4 and DMSO. The assay was developed after 3 days if not otherwise stated using CellTiter-Glo Luminescent Cell Viability Assay as described in the manufacturer's protocol. In brief, the plates were equilibrated to room temperature for 30 minutes. 100 mL CellTiter-Glo Buffer were added to the CellTiter-Glo Substrate Flask and mixed well. 30 μL of the reagent was transferred to each well. After incubation for 3 minutes at room temperature while shaking at 550 rpm, the plate was incubated for another 10 minutes at room temperature. The luminescence was read on an Envision reader. The evaluation was performed using GraphPad Prism 8 by normalization of cells treated with sample to untreated cells. The data were fitted with 4 point logistic curve to determine the IC₅₀ value.

On EGFR high expressing MDAMB468 cells, the BRD4-degrading PROTAC GNE987 as well as the EGFR-binding PROTAC-antibody conjugation C225-L328C-GNE987 (DAR=1.62) and the EGFR-targeting bispecific antibody aEGFRxMIC2 complexed with 50% GNE987 (1:1) had comparable potencies. The cytotoxic effects of GNE987 were suppressed by incubation with VH032-binding antibody MIC2 as well as the non-binding aHER2xMIC2+GNE987 (1:1) (FIG. 17). The bispecific antibodies without PROTAC had no effect on cell viability in the range of concentrations tested.

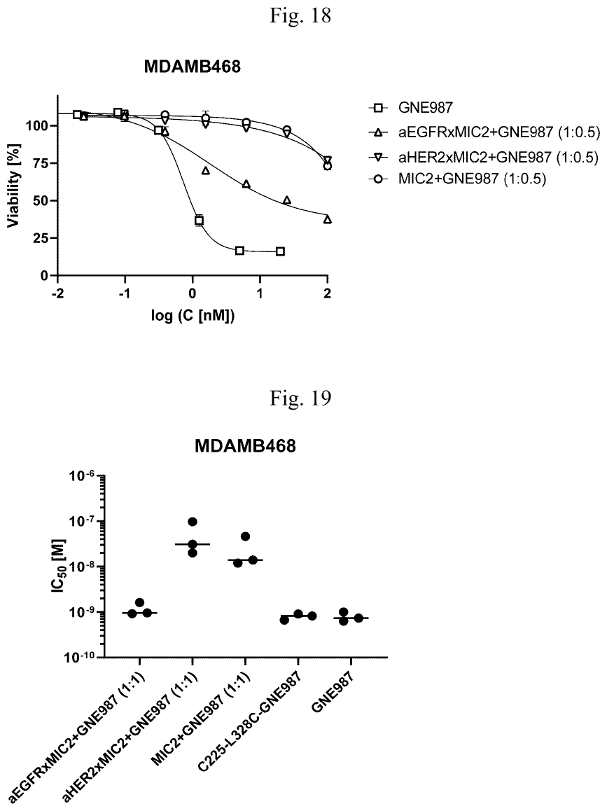

The reduction of the loading of the bispecific antibodies and anti-VH032 antibody MIC2 to 25% reduced the potency of the EGFR-targeting complex aEGFRxMIC2+GNE987 (1:0.5). At the same time, the impact on cell viability of the non-binding controls aHER2xMIC2 and MIC2 was also reduced at lowered loading (FIG. 18).

The potency of the non-binding control complex MIC2+GNE987 and the EGFR-targeting aEGFRxMIC2+GNE987 depends on the loading of the complexes (Table 4).

TABLE 4
IC₅₀-values of EGFR-binding aEGFRxMIC2 and non-binding control
MIC2 complexed with GNE987 at loadings of 25 and 50%.

| Molecule | Loading | IC₅₀ [nM] |
|---|---|---|
| aEGFRxMIC2 + GNE987 | 25% (1:0.5) | 3.4 |
| aEGFRxMIC2 + GNE987 | 50% (1:1) | 0.92 |
| MIC2 + GNE987 | 25% (1:0.5) | >100 |
| MIC2 + GNE987 | 50% (1:1) | 11.9 |

A selectivity index can be calculated by dividing the potency of the non-binding control complex by the EGFR-targeting aEGFRxMIC2+GNE987 complex for each respective loading. The selectivity index amounts to 12.9 for the 50% (1:1) loading and 29.4 for the 25% (1:0.5) loading.

The experiment was performed in biological triplicates. The potencies of the molecules are summarized in Table 5 and depicted graphically in FIG. 19.

TABLE 5
IC₅₀-values of EGFR-binding aEGFRxMIC2 + GNE987 complex
and controls on MDAMB468. The potencies and standard deviation
are derived from three independent experiments.

| Molecule | Loading | IC₅₀ [M] | STD [M] |
|---|---|---|---|
| aEGFRxMIC2 + GNE987 | 50% | 1.2E−09 | 3.3E−10 |
| (1:1) | | | |
| aHER2xMIC2 + GNE987 | 50% | 5.0E−08 | 3.4E−08 |
| (1:1) | | | |
| MIC2 + GNE987 (1:1) | 50% | 2.4E−08 | 1.6E−08 |
| C225-L328C-GNE987 | | 8.0E−10 | 1.0E−10 |
| GNE987 | | 7.9E−10 | 1.5E−10 |

The complexation of GNE987 with EGFR binding aEGFRxMIC2 leads to a 42-fold higher potency compared to a complex of the non-binding aHER2xMIC2 with GNE987 and to a 21-fold higher potency compared to the complex of non-binding MIC2 antibody with GNE987.

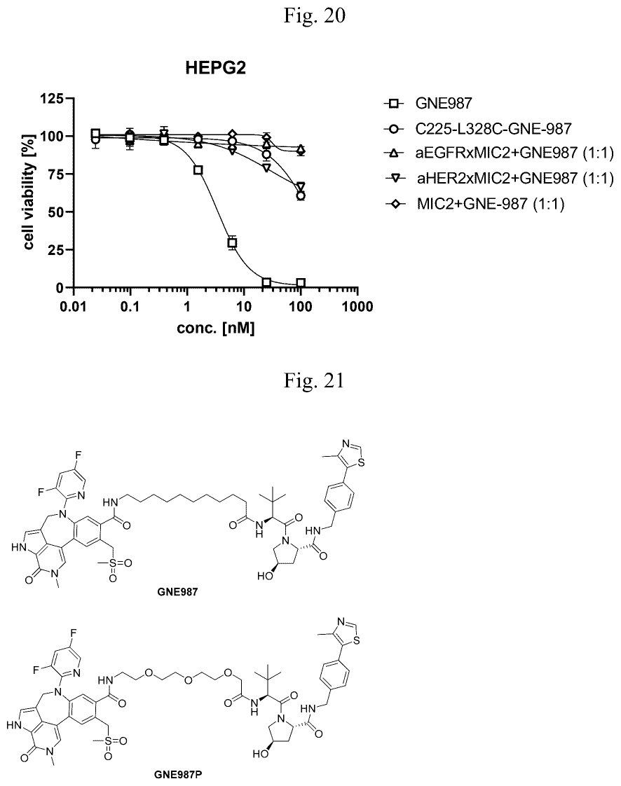

Additionally, aEGFRxMIC2 as well as aHER2xMIC2 complexed with 50% GNE987 was investigated on EGFR-negative cell line HEPG2 together with several benchmarks including the PROTAC GNE987 alone, a non-targeting MIC2+GNE987 complex and PROTAC-ADC targeting EGFR (C225_L328C-GNE987) (FIG. 20). While the PROTAC itself had strong antiproliferative effects on HEPG2 cells with an IC₅₀ value of 3.1 nM all antibody-based constructs had IC50 values >100 nM. aEGFRxMIC2 and MIC2 complexed with GNE987 suppressed toxicity of GNE987 the strongest and induced only minimal toxicity (˜10%) at 100 nM.

### Conclusion

The cell viability assay data underpin the BRD4 degradation data. A) The aEGFRxMIC2+GNE987 (50%) complex exhibits a similar cytotoxic effect on EGFR-expressing MDAMB468 cells compared to the PROTAC-ADC C225-L328C-GNE987 and the PROTAC GNE987. B) the antibody complexes that cannot enter the cells because they fully lack a targeting moiety (MIC2+GNE987 (50%)) or because the antibody's target receptor is not expressed on MDAMB468 (aHER2xMIC2+GNE987 (50%)) have decreased anti-proliferative effects. Furthermore, on HEPG2 cells that do not express EGFR, cytotoxicity of all antibody-based complexes and conjugates was decreased compared to the PROTAC alone which lacks a targeting moiety.

### Targeted Delivery of Additional PROTACs

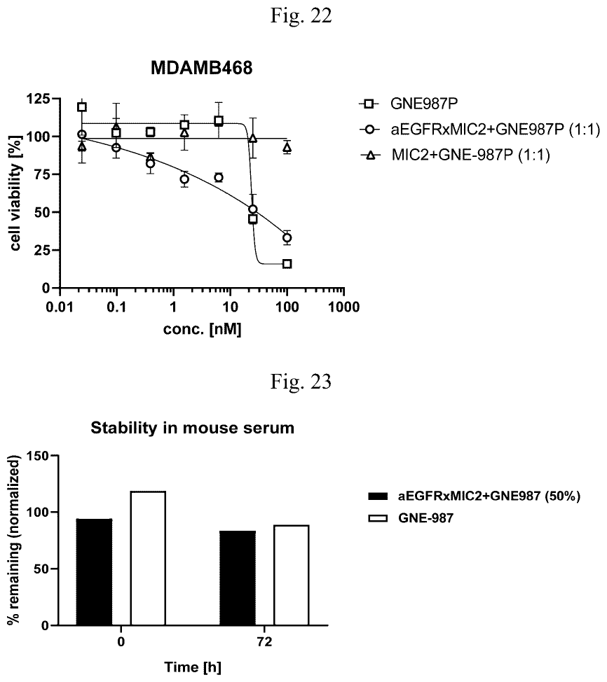

To demonstrate targeted delivery of another PROTAC, a GNE987 analogue possessing a hydrophilic PEG linker, GNE987P (FIG. 21), was complexed with MIC2 or aEGFRxMIC2 and incubated on EGFR-expressing MDAMB468 cells (FIG. 22). aEGFRxMIC2+GNE987P mediates increased cytotoxicity compared to GNE987P alone in a concentration range of 0.1-10 nM indicating targeted intracellular delivery of GNE987P by aEGFRxMIC2 while the non-binding control construct MIC2+GNE987P was significantly less toxic.

### Mouse Serum Stability

The antibody aEGFRxMIC2 was mixed with GNE987 so that the final concentrations were 40 UM each. The mixture was incubated 2 hours at room temperature while shaking at 650 rpm. 15% (vol/vol) 2 M HEPES buffer pH 7.55 were added to mouse serum from Biowest (Lot.no. S18169S2160) followed by sterile filtration.

aEGFRxMIC2+GNE987 and GNE987 was diluted to 5 μM using the mouse serum-HEPES mixture and incubated at 37° C. and 5% CO₂ for 0, 2, 4, 6, 24, 48, 72 and 96 hours. The incubation was stopped by freezing at −20° C.

The concentration of PROTAC GNE987 was quantified using LC-MS. GNE987 and GNE987 in the complex with aEGFRxMIC2 were stable over 72 hours (FIG. 23).

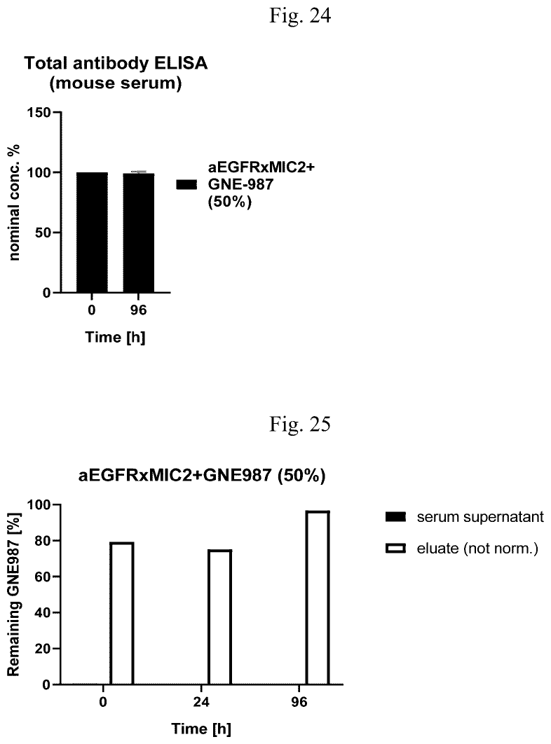

In addition, the concentration of intact aEGFRxMIC2 antibody was quantified in samples which were incubated in mouse serum for 96 hours. Quantification was performed using a total antibody ELISA (FIG. 24). The concentration of intact antibody was unaffected, demonstrating high plasma stability of the bispecific antibody.

In addition, aEGFRxMIC2 in complex with GNE987 was incubated in mouse serum for 0 and 96 hours and samples were subsequently subjected to an affinity capture assay. Therefore, beads were vortexed and transferred into 1.5 mL LoBind tube followed by washing with 500 μL HBS-E buffer three times. 0.2 μg/μL Biotin-SP (long spacer) AffiniPure Goat Anti-Human IgG (Fcγ fragment specific) were added to the beads and it was incubated for 2 h on a rotator. The beads were washed with 500 μL HBS-E buffer three times. 20 μL 0.5 μg/μL sample were diluted with HBS-E buffer to 0.1 μg/μL, added to the beads and incubated 2 h on a rotator. The supernatant is collected. 100 μL Acetonitrile was added to the beads followed by 30 min incubation at 1000 rpm and the eluate was collected. GNE987 was quantified from supernatant and eluate using LC-MS/MS.

No GNE987 was detected in the serum supernatant while the eluate still comprised 96.7% intact GNE987 that was bound to the antibody. Surprisingly, antibody-bound GNE987 could still be detected after 96 h in the eluate, indicating a high stability (FIG. 25).

### Storage Stability

Storage stability of antibody-PROTAC complexes was assessed after complexation (6 mg/ml, 650 rpm, 3 h, rt) by incubation in the fridge at 4° C. over 96 h as well as snap-freezing the complexes and storage at −80° C. for 24 h or at −20° C. at 96 h. Subsequently the samples were checked for visible changes, the polydispersity was assessed using DLS measurements and the loading was measured via SE-HPLC (Table 6). Samples with polydispersity up to 15% are considered monodisperse.

TABLE 6
Storage stability assessment of antibody-PROTAC
complexes in PBS pH 6.8, 5% DMSO final.

| | | | | DLS | DLS | SE-HPLC | |
|---|---|---|---|---|---|---|---|
| | t | T | Conc. | r_{H} | polydispersity | MIC2/DAR1/DAR2 | Visible |
| Sample | [h] | [° C.] | [μM] | [nm] | [% Pd] | ratio | evaluation |
| GNE987 alone | | | 82.4 | n.a. * | n.a. * | — | precipitation |
| MIC2 alone | | | 41.2 | 5.5 | 14.5 | 100/0/0 | clear |
| | | | (=6 | | | | solution |
| | | | mg/mL) | | | | |
| MIC2 + GNE987 | 0 | 25 | 41.2 / | 5.7 | 14.7 | 0/10/90 | clear |
| (100%) | | | 82.4 | | | | solution |
| MIC2 + GNE987 | 24 | 4 | 41.2 / | 7.0 | 12.2 | — | clear |
| (100%) | | | 82.4 | | | | solution |
| MIC2 + GNE987 | 96 | 4 | 41.2 / | 7.3 | 13.9 | 0/10/90 | clear |
| (100%) | | | 82.4 | | | | solution |
| MIC2 + GNE987 | 24 | −80 | 41.2 / | 6.8 | 15.5 | 0/13/87 | clear |
| (100%) | | | 82.4 | | | | solution |
| MIC2 + GNE987 | 96 | −20 | 41.2 / | 6.5 | 7.1 | 0/12/88 | clear |
| (100%) | | | 82.4 | | | | solution |

The data indicate not only high storage stability of the complexes since polydispersity remained largely unchanged as well as loading but also a shielding effect on PROTAC hydrophobicity was observed as hydrophobic GNE987 did not precipitate in the presence of MIC2

### VHH Based Antibodies and PAX

### Immunization

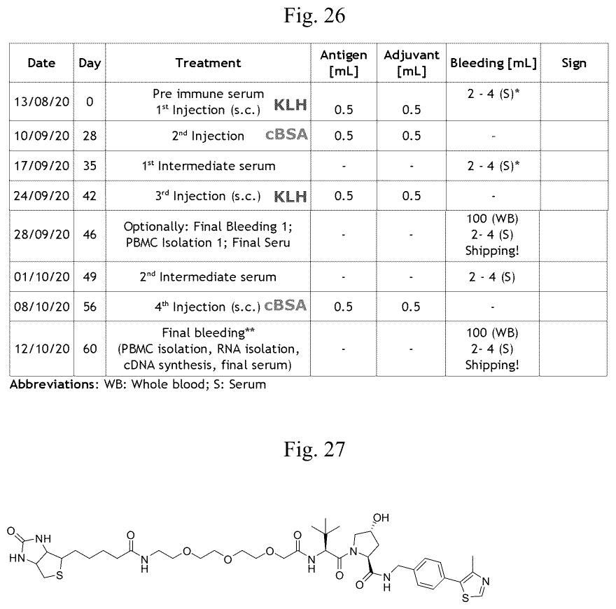

New world camelids (NWC) were immunized with alternating KLH-based and cBSA-based immunogens (schedule FIG. 26). Serum ELISA assays were performed with huFc-hapten conjugates to monitor the VHL ligand specific immune response. All animals showed an immune response after immunization.

### Antibody Gene Libraries

An immune library was generated from the immune repertoire of three immunized NWCs for the selection of VHL ligand specific antibodies. Peripheral Blood Mononuclear Cells (PBMCs) were isolated from the blood of the animals. RNA was extracted, purified, and used for cDNA synthesis. Then, the cDNA pool was used for the amplification of VHH gene sequences by PCR, cloned into a VHH antibody-phage display vector and used for the transformation of E. coli. The size of the antibody-gene library was determined by serial dilution and colony counting. Additionally, the insert-rate was determined by cPCR and the number of clones with a functional ORF determined by DNA-sequence analysis. Afterwards, the transformed bacteria were propagated and used for the packaging of antibody-phage particles. After purification of the antibody-phage particles, the presence of an antibody-pill fusion protein was checked by SDS-PAGE, western blotting and anti-pill immunoblot staining of the antibody-phage particles. An overview of the library characteristics is given below (Table 7):

TABLE 7
Library characteristics for antibody hit discovery campaign using phage display.

| | | | | Produced |
|---|---|---|---|---|
| | Library size | Insert-rate | Functional clones | antibody-phage |
| Library | [cfu] | [%] | [%] | [cfu] |
| New world camelid VHH | 1.44 × 10⁹ | 92 | 65 | 1.1 × 10¹² |
| library | | | | |

### Antibody Discovery

First, the libraries were cleared from unspecific or cross-reactive antibody-phages. For that purpose, the library was incubated in presence of immobilized streptavidin, magnetic streptavidin beads and BSA. Antibody-phage that bound to the negative antigens were removed from further selection.

After that, the cleared library was selected for target antigen specific antibodies. For that purpose, a conjugate of VH032 and a PEGylated crosslinker with pendant amine was acquired (Sigma-Aldrich; FIG. 27) and biotinylated via Biotin-NHS-ester coupling. The biotinylated VH032 was purified and analyzed by HPLC. First, the biotinylated VH032 was added to the cleared library preparations. Antibody-phage that bound to the biotinylated target antigen were captured and recovered from the solution using magnetic streptavidin beads. The beads were washed multiple times with a BSA solution (containing 0.05% Tween20) and PBS in order to remove unspecific or weakly bound antibody-phage particles. Antigen-specific antibody phage were eluted from the beads by trypsin treatment and rescued by E. coli infection. After short propagation, the bacteria were co-infected with M13K07 helperphage and antibody-phage amplification was induced. The amplified phages that originated from the immune libraries were used for two more selection cycles as described above.

### Antibody Screening

After the antibody-phage selection, the binding characteristics of the monoclonal antibody clones were analyzed by ELISA. For the immune libraries, the selection outputs after selection cycles two and three were used. 384 single clones were picked for VHH antibody expression in bacteria. The productions were tested for their binding specificity by ELISA on:
[symbol] Streptavidin+biotinylated VH032
[symbol] Streptavidin
[symbol] Human Fc-VHL-1
[symbol] Human Fc
Antibody clones were identified as antigen specific, if:
The ELISA binding signal to the positive antigens was ≥0.1
The ELISA binding signal to the negative antigens was ≤0.1
The signal-to-noise (S/N) ratio between positive and negative antigens was ≥10

All 562 Hits were used for DNA-sequence analysis to identify antibodies with a unique antibody sequence (≥1 amino acid difference in the CDRs). 113 unique clones were identified from the NWC library.

To identify clones with the best binding affinity, a BLI off-rate measurement was performed. First, VHH containing culture supernatants were produced of unique clones. After that, the association and dissociation of the antibody fragment to the biotinylated VHL was measured which was immobilized onto BLI Streptavidin sensors. The binding curve was fitted with a 1:1 binding model and the dissociation rate calculated.

Based on the off-rate and antibody sequence information, 10 lead clones (named MIC5-MIC14) were selected for conversion into the final format.

### Antibody Conversion

The VHH genes were amplified from the phagemid DNA by PCR and cloned into two different IgG expression vectors. One expression vector encoded the EGFR-targeting Cetuximab IgG antibody. The other expression vector encoded the CD33-targeting Gemtuzumab IgG antibody. To insert the VHH antibody fragment into the expression vector, it was genetically fused to the C-terminus of the IgG antibody's heavy chain. The antibody fragment was separated from the IgG heavy chain via a short GS-Linker (GSGGGSGGSGGGGSG (SEQ ID NO: 28)), generating the following format: HC-Linker-VHH.

After sequence verification and preparation of transfection-grade DNA, HEK cells were transiently transfected with the expression vectors of one VHH clone. The antibodies were produced by the HEK cells and secreted into the culture medium for 7 days. After clearance of the culture supernatant from cells by centrifugation, the IgG antibodies were purified by protein A affinity chromatography. After adjustment of the buffer to PBS, the protein concentration of the antibodies was determined by UV/VIS spectrometry. Integrity and purity of the antibodies was assessed by SDS-PAGE under reducing conditions. The functional binding activity of the antibodies to the target antigen was measured by ELISA.

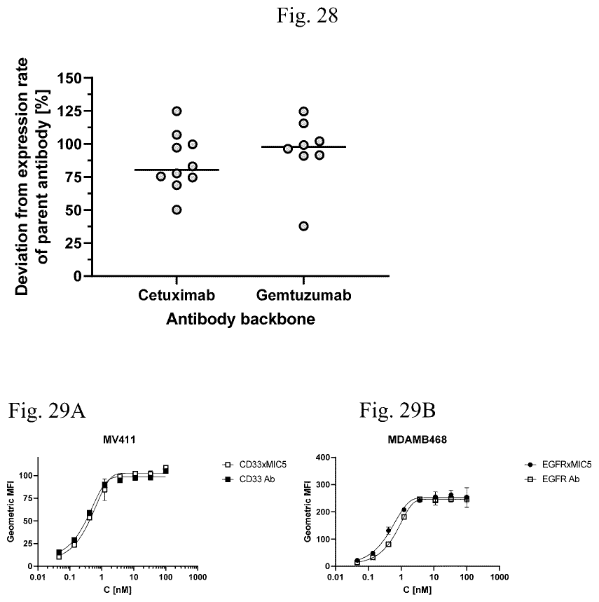

Additionally, the parent antibody was produced using the same procedure to be able to compare expression rates of the parent antibody versus fusion proteins. The data are depicted exemplarily in FIG. 28 for the VHH fusions to cetuximab and gemtuzumab demonstrating no major impact of the VHH fusion on producibility.

### Versatility of VHH Binding to PROTACs

The kinetic and affinity parameters of protein-PROTAC interactions were evaluated by SPR. The anti-PROTAC VHH clones MIC5-MIC14 were immobilized as CD33 or CLL1 antibody fusion (the manufacturing of fusion proteins and the used linker is described in chapter 7.9) onto a CM5 (Series S) sensor chip via the standard amine coupling procedure, at 25° C. Prior to immobilization, the carboxymethylated surface of the chip was activated with 400 mM 1-ethyl-3-(3-dimethylaminopropyl)-carbodiimide and 100 mM N-hydroxysuccinimide for 7 min. Hit anti-PROTAC VHH as CD33 and CLL1 antibody fusions were diluted to 10 μg/ml in 10 mM Acetate at pH 4.5 and immobilized on the activated surface chip for 7 min, in order to reach 3,000 to 6,000 response units (RU). The remaining activated carboxymethylated groups were blocked with a 10 min injection of 1 M ethanolamine pH 8. HBS-N, which consists of 10 mM HEPES pH 7.4 and 150 mM NaCl, was used as the background buffer during immobilization. PROTACs were prediluted in DMSO, diluted 1:50 in running buffer (12 mM phosphate pH 7.4, 137 mM NaCl, 2.7 mM KCl, 0.05% Tween20, 2% DMSO) and injected at ten different concentrations using two-fold dilution series, from 1 μM to 0.002 μM. A DMSO solvent correction (1%-3%) was performed to account for variations in bulk signal and to achieve high-quality data. Interaction analysis cycles consisted of a 300 sec sample injection (30 μL/min; association phase) followed by 900 sec of buffer flow (dissociation phase).

All sensorgrams were processed by first subtracting the binding response recorded from the control surface (reference flow-channel), followed by subtracting of the buffer blank injection from the active flow-channel (target protein immobilized). All datasets were fit to a simple 1:1 Langmuir interaction model to determine the kinetic rate constants. Experiments were performed on a Biacore 8k+ (Cytiva, Uppsala, Sweden) at 25° C. and the interactions were evaluated using the provided Biacore Insight Evaluation software.

The results are summarized in Table 8. The PROTACs for this study were selected on the basis of their chemical structure to test a set of molecules as diverse as possible. In general, all antibodies bound a range of PROTACs with subnanomolar to triple-digit nanomolar affinities.

From all variants tested, the MIC7 anti-PROTAC VHH had the most favourable binding profile binding to the vast majority of PROTACs with single-digit nanomolar to even subnanomolar affinities. The MIC7 clone tolerates all PROTACs where the linker to the protein binding moiety exits in R1 and R2 however not in R3. With regard to the linker chemistry, there was no negative impact on MIC7-PROTAC binding of any sort observed. Additionally, MIC7 tolerates the common methyl group in R4. Finally, SPR PROTAC binding measurements with a CLL1-binding antibody MIC7 VHH fusion (CLL1xMIC7) instead of the CD33-binding antibody fusion indicate that PROTAC binding is not impacted by fusion to other antibodies as binding affinities were not affected (Table 8).

TABLE 8
Affinities (K_{D}) of VHH clones to PROTACs determined using SPR. The VHHs were
studied as antibody fusion proteins by C-terminal addition to the heavy chain of either an anti-
CD33 or anti-CLL1 antibody. N/D-not detected (complete PROTACS structures can be found
in FIGS. 8A-8B).
PROTAC

| | Exit | Variations | | | | | | | | | |
|---|---|---|---|---|---|---|---|---|---|---|---|
| PROTAC | vector | in VH032 | Linker | | | | | | | | |
| GNE [symbol] 87 | R1 | — | 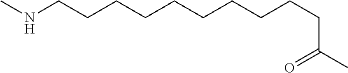 | | | | | | | | |
| GNE [symbol] 87P | R1 | — | 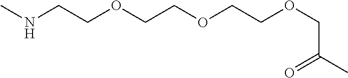 | | | | | | | | |
| ARV771 | R1 | R4 = Me | 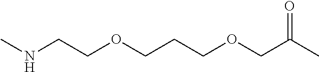 | | | | | | | | |
| BETTY2 | R1 | — | 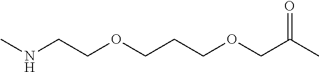 | | | | | | | | |
| BETTY3 | R1 | R3 = OH | 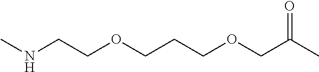 | | | | | | | | |
| cMETd1 | R1 | R5 = H, R6 = OH | 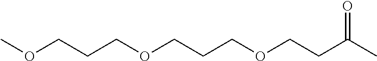 | | | | | | | | |
| AT1 | R2 | R1 = Acyl | 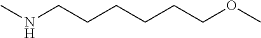 | | | | | | | | |
| SIM1 | R1 | — | 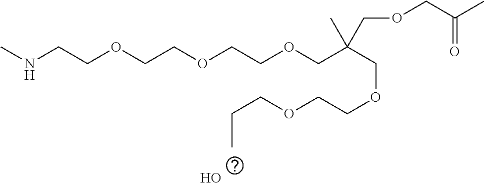 | | | | | | | | |
| SIA [symbol] S178 | R1 | — | 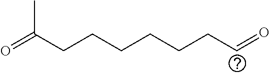 | | | | | | | | |
| ACB [symbol] | R3 | R1 = cyclopropyl (F) | 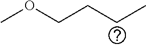 | | | | | | | | |
| | K_{D} [M] for antibody binding to PROTAC | | | | | | | | | | |
| | CD33 | CD33 | CD33 | | CD33 | CD33 | CD33 | CD33 | CD33 | CD33 | CD33 |
| | xMIC | xMIC | xMIC | CLL1x | xMIC | xMIC | xMIC | xMIC | xMIC | xMIC | xMIC |
| PROTAC | 5 | 6 | 7 | MIC7 | 8 | 9 | 10 | 11 | 12 | 13 | 14 |
| GNE [symbol] 87 | 9.6E−10 | 4.1E−08 | <1E−10 | <1E−10 | 6.5E−09 | 4.3E−08 | 1.3E−08 | 2.0E−08 | 4.2E−08 | 2.3E−08 | 1.0E−08 |
| GNE [symbol] 87P | 5.7E−09 | 1.5E−08 | 2.9E−09 | 9.1E−10 | 5.6E−09 | 5.0E−09 | 4. [symbol] E−09 | 3.2E−09 | 6.0E−09 | 2. [symbol] E−09 | 2.9E−09 |
| ARV771 | 2.7E−07 | 5.3E−08 | 4.2E−09 | 8.8E−09 | 3.1E−08 | 4.0E−09 | 1.4E−07 | 3.7E−09 | 5.6E−09 | 1.3E−08 | 1.4E−07 |
| BETTY2 | — | — | 2.1E−09 | — | — | — | — | — | — | — | — |
| BETTY3 | 7.4E−07 | 9.0E−07 | 3.3E−09 | — | 3.7E−07 | 7.5E−08 | 5.2E−07 | 2.0E−08 | 1.7E−08 | 1.1E−07 | 1.9E−07 |
| cMETd1 | 9.2E−08 | 2.1E−07 | 1.2E−08 | — | 1.2E−07 | 7.6E−07 | 6.7E−08 | 3.5E−08 | 1.0E−07 | 4.4E−08 | 6.1E−08 |
| AT1 | 2.4E−09 | 7.3E−09 | 2.1E−09 | — | 5.2E−09 | 1.4E−09 | 2.4E−09 | 1.9E−10 | 1.7E−09 | 1.7E−09 | 2.9E−09 |
| SIM1 | — | — | 3.8E−09 | <1E−10 | — | — | — | — | — | — | — |
| SIA [symbol] S178 | — | — | 8.3E−09 | — | — | — | — | — | — | — | — |
| ACB [symbol] | 2.5E−06 | 6.5E−06 | N/D | N/D | N/D | N/D | 8.5E−06 | 1.1E−04 | N/D | 9.3E−05 | 9.3E−07 |

[symbol]

indicates data missing or illegible when filed

### Manufacturing of VHH-Based PAX

VHH antibody fragments were fused to heavy and light chains of IgG-type antibodies to allow delivery of PROTACs to various disease-relevant cell lines. The fusions were made as follows: the antibody's heavy or light or heavy and light chain were elongated c-terminally by the linker (GSGGGSGGSGGGGSG (SEQ ID NO: 28)) followed by the sequence of the VHH (e.g., YU734-F06 (MIC7)). This way, bispecific antibodies can be generated as described in FIG. 3 that are able to recognize a cell surface receptor and simultaneously bind to the VHL-ligand of PROTACs. The linker-VHH (linker: GSGGGSGGSGGGGSG (SEQ ID NO: 28)) sequences can be fused multiple times as repeating units (such as linker-VHH-linker-VHH, i.e. two repeats) to the HC, LC or both. A maximum of 3 linker-VHHs per chain were either fused to the HC, LC or to both. The nomenclature is as follows: A CD33 targeting antibody to which one MIC7 VHH was fused via the above-mentioned linker to the C-terminus of each heavy chain is named CD33xMIC7. This is the only exception to the following nomenclature, which was used for all other constructs: Here, the generalized formula: CD33xMIC7_{NHML} applies, where N and M are 2, 4, 6. The “H” indicates that the fusion was made at the heavy chain, the “L” indicates a fusion to the LC. If no VHHs are fused to HC or LC, the respective letter disappears. For example, a CD33 targeting antibody to which two MIC7 VHHs (linker-VHH-linker-VHH, i.e. two repeats) were fused to the C-terminus of each heavy chain as well as to the C-terminus of each light chain is called CD33xMIC7_{4H4L}. The antibody backbones used for generating the bispecific fusions proteins and backbone alterations are depicted in Table 9. Table 10 shows the nomenclature of the PAX targeting CD33, by way of example.

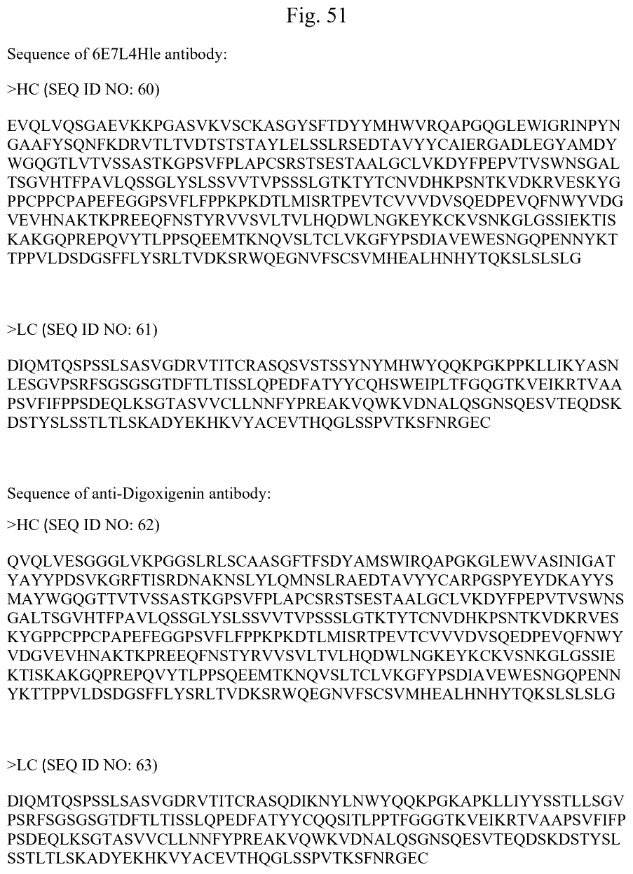

TABLE 9
IgG-type antibody backbones for fusion
with VHH antibody fragments.

| Target | CDR origin | Alterations |
|---|---|---|
| CD33 | Gemtuzumab | Isotype: IgG4 |
| | | Heavy chain mutations: |
| | | P329G, L235E |
| EGFR | Cetuximab | Isotype: IgG4 |
| | | Heavy chain mutations: |
| | | P329G, S228P, L235E |
| CLL1 | 6E7L4Hle (sequences shown | — |
| | in FIG. 51) | |
| NAPI2B | Upifitamab | — |
| B7H3 | Omburtamab | — |
| HER2 | Trastuzumab | — |
| TROP2 | Sacituzumab | — |
| Digoxigenin | Sequences shown in FIG. 51 | — |

The antibody production was performed as described in chapter 7.2. Complexation to create PAX was performed as follows: 10 μM (final) antibody-VHH fusion were mixed in PBS pH 7.4 with VHL-based PROTAC in DMSO to achieve the desired loading (Table 3). PAX determined for in vivo application were complexed at an antibody concentration of 68.8 μM.

For dispensing, 5% Tween-20 in PBS pH 7.4 solution was added to a final concentration of 0.3%. The samples were incubated on a ThermoMixer for 3 h at 25° C. while shaking at 650 rpm.

TABLE 10
Nomenclature for PAX targeting CD33.

| | | Number of | | |
|---|---|---|---|---|
| | | Linker-VHH | | |
| Target | Chains | HCs | LCs | Name |
| CD33 | unaltered LC + HC-Linker VHH (MIC5) | 2 | — | CD33xMIC5 |
| | unaltered LC + HC-Linker VHH (MIC7) | 2 | — | CD33xMIC7 |
| | unaltered LC + HC-Linker-VHH-Linker- | 4 | — | CD33xMIC7_{4H} |
| | VHH (MIC7) | | | |
| | unaltered LC + HC-Linker-VHH-Linker- | 6 | — | CD33xMIC7_{6H} |
| | VHH-Linker-VHH (MIC7) | | | |
| | LC-Linker-VHH (MIC7) + unaltered HC | — | 2 | CD33xMIC7_{2L} |
| | LC-Linker-VHH-Linker-VHH (MIC7) + | — | 4 | CD33xMIC7_{4L} |
| | unaltered HC | | | |
| | LC-Linker-VHH-Linker-VHH-Linker-VHH | | 6 | CD33xMIC76_{L} |
| | (MIC7) + unaltered HC | | | |
| | LC-Linker-VHH (MIC7) + HC-Linker VHH | 2 | 2 | CD33xMIC7_{2L2H} |
| | (MIC7) | | | |
| | LC-Linker-VHH-Linker-VHH (MIC7) + | 4 | 4 | CD33xMIC7_{4L4H} |
| | HC-Linker VHH-Linker-VHH (MIC7) | | | |
| | LC-Linker-VHH-Linker-VHH-Linker-VHH | 6 | 6 | CD33xMIC7_{6L6H} |
| | (MIC7) + HC-Linker-VHH-Linker-VHH- | | | |
| | Linker-VHH (MIC7) | | | |

### Cellular Characterization of VHH-Based PAX

### Cellular Profiling of VHL-Based PROTAC Binding Antibody Fragments as Fusions of Cetuximab and Gemtuzumab

Gemtuzumab- and cetuximab-based VHH fusions were created by genetically attaching the VHH c-terminally via a linker (GSGGGSGGSGGGGSG (SEQ ID NO: 28)) to the HCs of the respective antibody as described in chapter 7.9. After expression and purification, the antibody fusion proteins were loaded with GNE987 in a 1:1 ratio (50% loading). This way, 10 VHH antibody fragments could be characterized regarding their ability to induce cell killing (as described in chapter 7.10.5) on target-positive cells or to prevent non-selective uptake into non-targeted cells. The results are depicted in Table 11. As a metric of selectivity, selectivity indices were introduced. Firstly, a selectivity index is obtained that allows comparison of constructs in the same cellular context by dividing the potency of gemtuzumab-based constructs by the potency of cetuximab-based constructs. In this case, the clones MIC5-MIC8 had the highest selectivity indices which proves that those constructs bind to the PROTAC during a 3-day incubation time in cell medium. Secondly, the potency of cetuximab-based constructs on EGFR-negative HEPG2 cells was divided by the potency of the same constructs on EGFR-positive MDAMB468 cells which is a measure of selectivity mediated by receptor-expression dependent uptake. In this case, the constructs MIC7-MIC10 yielded the highest selectivity indices. One additional parameter that is indicative of high selectivity is the potency of the fusion proteins loaded with PROTAC on HEPG2 cells, where no cytotoxicity was expected. The PROTAC alone has an IC50 value of 2.6 nM on HEPG2. This indicates that the PROTAC is released already outside of the cells leading to potent cell killing. In contrast to that, the VHH-based constructs led to strong detoxification effects with potencies >100 nM. Overall, the clones MIC5, MIC7 and MIC10 exhibited a promising profile, especially when taking the affinities (chapter 7.8.6) into account.

TABLE 11
Cellular profiling of CD33-binding gemtuzumab (G)- and EGFR-binding
cetuximab (c)-based VHH fusion proteins combined with PROTAC GNE987
on EGFR-positive MDAMB468 cells and MDAMB468-negative HEPG2 cells.
IC50 values were used to calculate selectivity indices.

| | | | IC50 [M] of cetuximab | | Selectivity Index | |
|---|---|---|---|---|---|---|
| | | | (C)-based fusion proteins | | | IC50 of C |
| Clone | | | complexed with GNE987 (1:1) | | IC5O(G)/ | on HEPG2/ |
| Type | ID_A | ID_B | MDAMB468 | HEPG2 | IC50(C) | MDAMB468 |
| VHH | YU733-G10 | MIC5 | 1.8E−10 | — | 98.1 | — |
| VHH | YU734-A12 | MIC6 | 1.7E−10 | — | 12.5 | — |
| VHH | YU734-F06 | MIC7 | 1.8E−10 | 1.0E−07 | 20.9 | 565.3 |
| VHH | YU737-C12 | MIC8 | 1.9E−10 | — | 15.5 | |
| VHH | YU736-C11 | MIC9 | 1.4E−09 | 1.0E−07 | — | 70.7 |
| VHH | YU737-D07 | MIC10 | 6.7E−10 | 1.0E−07 | — | 150.1 |
| VHH | YU732-G09 | MIC11 | 2.5E−10 | — | 2.6 | — |
| VHH | YU732-G11 | MIC12 | 3.1E−10 | — | 1.6 | — |
| VHH | YU732-G12 | MIC13 | 2.3E−10 | — | 2.3 | — |
| VHH | YU736-A10 | MIC14 | 1.9E−10 | — | 9.3 | — |

Cell Binding of Backbone Antibody is not Impacted by VHH Fusion and Loading of PAX with PROTAC

The impact on cell binding of the VHH fusion via the linker described in chapter 7.9 (GSGGGSGGSGGGGSG (SEQ ID NO: 28)) to either a CD33-binding antibody Gemtuzumab (IgG4-PG-SPLE) or a EGFR-binding antibody Cetuximab was investigated using flow cytometry. Therefore, the binding of CD33xMIC5 and CD33 Ab without VHH MIC5 fusion to CD33-expressing MV411 cells was assessed. 1×10⁵ MV411 or MDAMB468 cells were seeded in round bottom 96 well plates, washed three times with PBS pH 7.4 containing 1% (w/V) bovine serum albumin (BSA) followed by incubation with the respective antibody for 30 min on ice. Subsequently, cells were washed three times with PBS, pH 7.4, 1% (w/V) BSA and incubated with fluorescently labeled secondary antibody Alexa Fluor 488 AffiniPure Fab Fragment Goat Anti-Human IgG (H+L) for 30 min on ice. Subsequently, cells were washed three times with PBS, pH 7.4, 1% (w/v) BSA. Cells were analyzed by flow cytometry on an Intellicyt IQue3 Screener and analyzed via IntelliCyt ForeCyt Enterprise Client Edition 8.0 (R3) software. Cells were cultured in RPMI-1640+10% FCS. The direct comparison demonstrated that the addition of the VHH MIC5 had no impact on cellular binding (FIGS. 29A-B).

Subsequently, it was investigated to what extent the loading of the antibody-VHH fusions with PROTAC influenced the cellular binding behavior. Therefore, cellular binding to HL60, MOLM13, MV411, RAMOS and U937 was determined for the CD33-binding antibody-VHH fusion CD33xMIC7. The binding of the same molecule loaded with 90% GNE987 was assessed. As an isotype non-binding control, a digoxigenin binding antibody was used. Prior to use for cell staining, CD33xMIC7 was incubated for 3 h at 25° C. and shaking at 650 rpm in PBS with 1% FCS with 5% DMSO-dissolved GNE987 at 1.8-fold molar excess. Isotype non-binding control was analyzed upon incubation with 5% DMSO. From each cell line, 200,000 cells per condition were taken, centrifuged, and incubated with the respective antibody, antibody-VHH fusion or PAX conditions in 200 μl PBS with 1% FCS at a concentration of 10 μg/ml for 45 min at 4° C. After quenching and one wash step with PBS with 1% FCS, samples were subsequently treated with fluorescein-labelled antihuman antibody #607 (1:50) for another 45 min. After quenching with PBS with 1% FCS, cells were resuspended in PBS containing 1 μg/mL propidium iodine (PI) staining dead cells to be excluded during analysis performed on a Becton Dickinson FACSCalibur flow cytometer. Quantitative analyses were done using the Flowing software from Turku Bioscience. Cells were cultured in RPMI-1640+10% FCS.

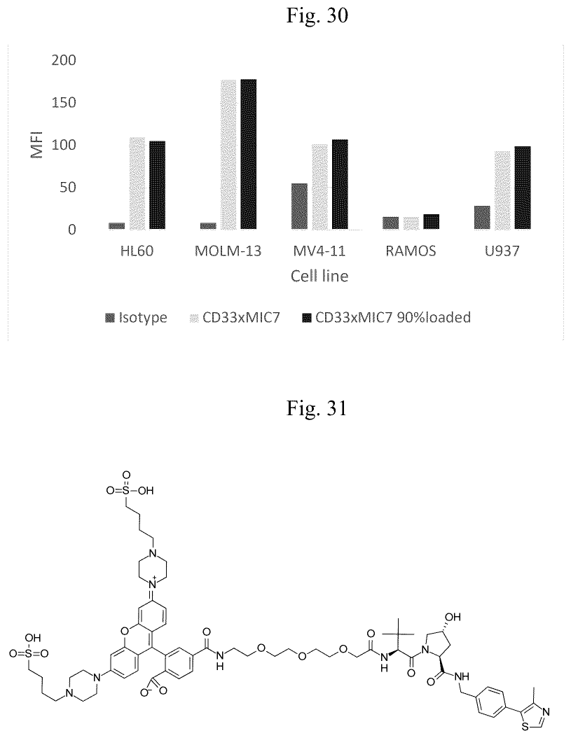

The loading of the antibody-VHH fusion with PROTAC had no impact on the binding as demonstrated in FIG. 30. The isotype control antibody showed strongly reduced binding to the cell line panel.

CD33 PAX (with pHAb Dye) Internalizes into CD33 Positive Cells

To elucidate if PAX uptake is mediated by receptor mediated endocytosis, CD33xMIC7 was loaded with a pH responsive VH032-pHAb dye (FIG. 31). VH032-pHAb dye is a pH sensor that exhibits only minimal fluorescence at pH >7, but significantly enhanced fluorescence at acidic pH. Therefore, trafficking of CD33xMIC7+VH032-pHAb dye to the acidic endosomal and lysosomal vesicles, upon receptor-mediated internalization, should result in enhanced fluorescence signals. Prior to use for cell staining, CD33xMIC7 was incubated for 2 h at 25° C. at 650 rpm in PBS with 5% DMSO-dissolved VH032-pHAb dye at 1.8-fold molar excess in the dark. CD33-positive MOLM13, MV411, U937 and receptor negative RAMOS cells were treated with the resulting PAX or with VH032-pHAb dye alone, as control. From each cell line, 200,000 cells per condition were taken, centrifuged, and incubated with the PAX in 200 μL PBS with 1% FCS at a concentration of 10 μg/ml for 6 h at 37° C. and shaking at 650 rpm in the dark. As a control, cells were treated with an equimolar concentration of VH032-pHAb dye alone. After one wash step with PBS with 1% FCS, cells were resuspended in 400 μl PBS with 1% FCS and cytometric analysis was performed on a Becton Dickinson FACSCalibur flow cytometer. Staining of dead cells with propidium iodine (PI) was not performed to omit interference with VHL-pHAb dye. Quantitative analyses were done using the FlowJo from BD biosciences. Cells were cultured in RPMI-1640+10% FCS.

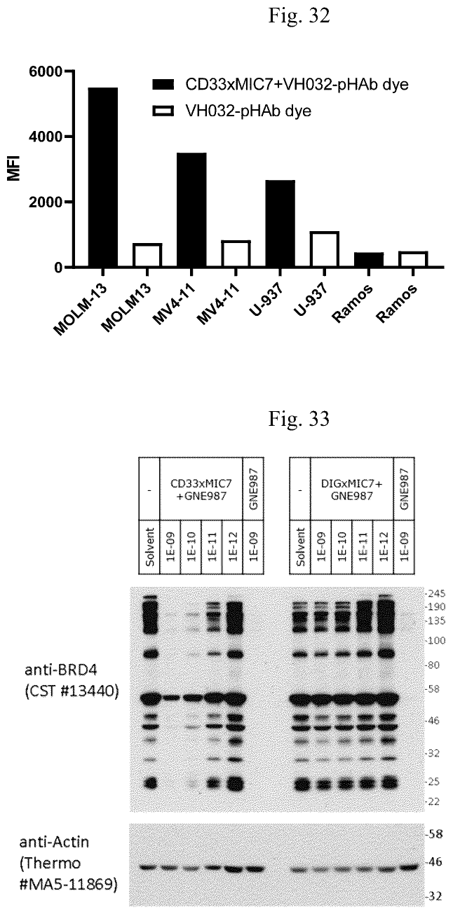

Enhanced fluorescence signals, compared to the control, for cells treated with CD33xMIC7+VH032-pHAb dye were found on all CD33-positive cell lines, whereas no enhanced mean fluorescence intensity was found for receptor-negative RAMOS cells (FIG. 32). These results demonstrated that PAX are taken up via receptor-mediated antibody internalization.

### CD33xMIC7+GNE987 PAX Induces BRD4 Degradation in Targeted Cells

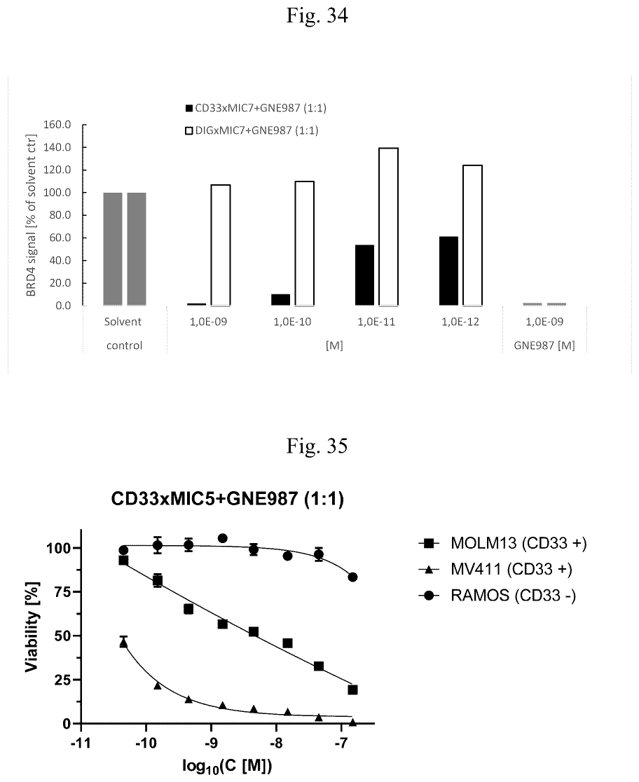

To demonstrate that PAX can mediate degradation of a target protein in targeted cells, BRD4 Western blot degradation assays were performed (FIG. 33 and FIG. 34). Therefore, CD33-positive MV411 cells were treated with CD33xMIC7+GNE987 or DIGxMIC7+GNE987, as non-binding PAX control, at different concentrations and GNE987 alone as control. MV411 cells were cultured in RPMI-1640 medium supplemented with 10% FCS and penicillin-streptomycin. MV411 cells were seeded in 12-well plates at 1 million cells/ml in 2 ml culture medium and cultured overnight in cell incubator at 37° C. and 5% CO₂. Complexation was performed as described in chapter 7.4. GNE987 only was preincubated at identical conditions but in absence of antibody. Subsequently, cells were treated by nanodrop dispersion using a Tecan dispenser with either CD33xMIC7+GNE987 (1:1), DIGxMIC7+GNE987 (1:1) or GNE987 in the respective concentration. All conditions were normalized to 0.0005% (v/V) DMSO and 3.0E-5% Tween20. Cells were incubated for 24 h in cell incubator at 37° C. and 5% CO₂. Treated cells were collected, washed in PBS and cell pellets lysed in cell lysis buffer (20 mM TRIS pH7.4, 100 mM NaCl, 1 mM EDTA, 0.5% TritonX-100) supplemented with Roche complete protease inhibitor mixture. After incubation for 10 min on ice, crude lysates were cleared via centrifugation at 15,000×g, 4° C. and cleared lysates were precipitated by acetone. Dried pellets were dissolved in SDS-loading buffer. Protein concentration was determined via Mettler Toledo UV5Nano photometer. Samples (50 μg/lane) were applied to 4-12% Bis-Tris SDS-PAGE gels (Thermo Fisher Scientific). After gel run, the samples were transferred to nitrocellulose membranes (Sigma Aldrich), the membranes were blocked with 5% skim milk in 0.1% TBS-Tween20 and incubated with indicated primary antibodies (Table 12).

TABLE 12
Primary antibodies used for Western Blot analysis.

| | | | working | |
|---|---|---|---|---|
| target | host | supplier | dilution | buffer |
| BRD4 | rabbit | Cell Signaling | 1:1000 | 5% skim milk, |
| | | Technology | | 0.1% TBS-T |
| actin | mouse | Thermo Fisher | 1:10.000 | 5% skim milk, |
| | | | | 0.1% TBS-T |

After incubation with corresponding HRP-labeled anti-rabbit/mouse secondary antibodies (GE-Healthcare) at 1:10,000 dilution, blots were detected using ECL solution (Advansta) and x-ray films (GE-Healthcare). Results were analyzed with ImageJ software (version 1.53K, NIH). Background subtracted BRD4 signals were normalized to corresponding actin signals and normalized BRD4 signal of solvent control was set 100%.

As expected BRD4 degradation was found for treatment with GNE987 alone. Additionally, concentration dependent BRD4 degradation was observed for CD33xMIC7+GNE987 whereas no degradation was observed for non-binding PAX control DIGxMIC7+GNE987 at all concentrations (FIG. 33 and FIG. 34). These results demonstrated that PAX are able to mediate concentration dependent and cell-type (target receptor) selective degradation of the intracellular protein of interest.

### CD33xMIC5+GNE987 PAX Induces Cytotoxic Effects Dependent on Receptor Expression and PAX Loading

Several cell viability experiments were performed following the procedure described below: Cells were seeded in different media into white cell culture-treated flat and clear bottom multiwell plates followed by overnight incubation in a humid chamber at 37° C. and 5% CO₂. Complexation was performed as described in chapter 7.4.

The solutions were added to the cells based on the antibody concentration using nanodrop dispension using a Tecan D300e Digital Dispenser and all wells were normalized to the same volume using 0.3% TW20 in PBS pH 7.4 and DMSO to a final solvent concentration of 0.05% DMSO and 0.003% TW20. Incubation was performed at 37° C. at 5% or 10% CO₂ dependent on the medium. The assay was developed after 3 days if not otherwise stated using CellTiter-Glo Luminescent Cell Viability Assay as described in the manufacturer's protocol. In brief, the plates were equilibrated to room temperature for 30 minutes. 100 mL CellTiter-Glo Buffer were added to the CellTiter-Glo Substrate Flask and mixed well. 30 μL of the reagent was transferred to each well. After incubation for 3 minutes at room temperature while shaking at 550 rpm, the plate was incubated for another 10 minutes at room temperature. The luminescence was read on an Envision reader from Perkin Elmer. Solvent alone and Bortezomib (1.0E-05 M) served as high control (100% viability) and low control (0% viability), respectively. Raw data were converted into percent cell viability relative to the high and low control, which were set to 100% and 0%, respectively. IC50 calculation was performed using GraphPad Prism software with a variable slope sigmoidal response fitting model using 0% viability as bottom constraint and 100% viability as top constraint. The concentration corresponds to the concentration of PROTACs in the PAX.

It was investigated if and to what extent the PAX technology can mediate cell-selective targeting of cells based on their cell surface receptor expression. Therefore, a CD33-targeting PAX was created by genetically fusing MIC5 to the HCs followed by loading the PROTAC GNE987. Then the PAX was tested on CD33-positive and CD33-negative cells.

The treatment of CD33-positive MV411 and MOLM13 cells and CD33-negative RAMOS cells with a serial dilution of CD33xMIC5 pre-loaded with 50% GNE987 led to decrease of viable cells after the 3 day incubation in case of MV411 and MOLM13 cells but not RAMOS cells (FIG. 35). This demonstrates that CD33xMIC5 mediates the uptake of the GNE987 PROTAC into receptor-positive cells while it prevents uptake into receptor-negative cells and hence the PAX was able to selectively deliver PROTAC into the targeted cells.

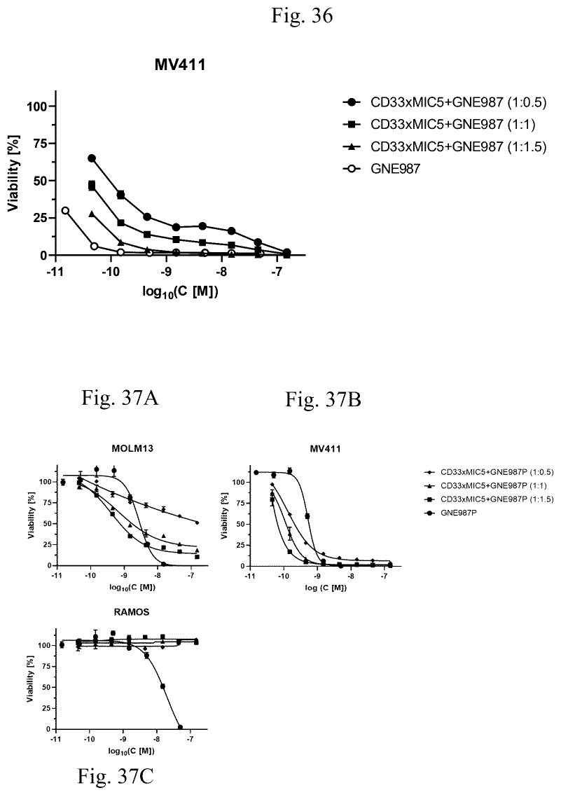

The cytotoxicity of the CD33xMIC5 constructs could be modulated by in- or decreasing the loading with PROTAC GNE987 (FIG. 36). The cytotoxicity of CD33xMIC5 combined with GNE987 on MV411 cells can be increased when the loading is increased from 25% to 50% or even 75%. This demonstrates that it is possible to tailor the cell-killing properties of the PAX by adjusting the amount of loaded PROTAC as wished.

Complexation of CD33xMIC5 with GNE987P can Improve Cell-Killing Potency of PROTAC GNE987P Alone

So far, it was shown that the PAX technology can be leveraged to deliver the PROTAC GNE987 to target cells and it was unclear if it is also suitable for other PROTACs. Therefore, another PROTAC was selected for further studies, namely GNE987P, and it was investigated if the PAX technology can mediate selectivity to this PROTAC, too.

Therefore, CD33-positive MV411 and MOLM13 as well as CD33-negative RAMOS cells were treated with CD33xMIC5+GNE987P with 25 to 75% loading and PROTAC GNE987P alone (FIG. 37A-37C). On both CD33-positive cell lines, the combination of CD33xMIC5+GNE987P was more potent than the PROTAC GNE987P alone and the potency of CD33xMIC5+GNE987P was dependent on the loading where higher loading led to higher potency. Overall, on CD33-negative cells no toxicity was observed up to 150 nM. It can be concluded, that the concept of facilitating targeted delivery of PROTACs using antibody-complexation might be more generalizable, since a second PROTAC was delivered selectively to CD33-expressing cells. Additionally, it was observed that the technology offers the possibility to even improve the potency of certain PROTACs by targeted delivery into the cells.

### CD33xMIC5 can be Used to Deliver an FLT3 Degrader to CD33-Positive Cells

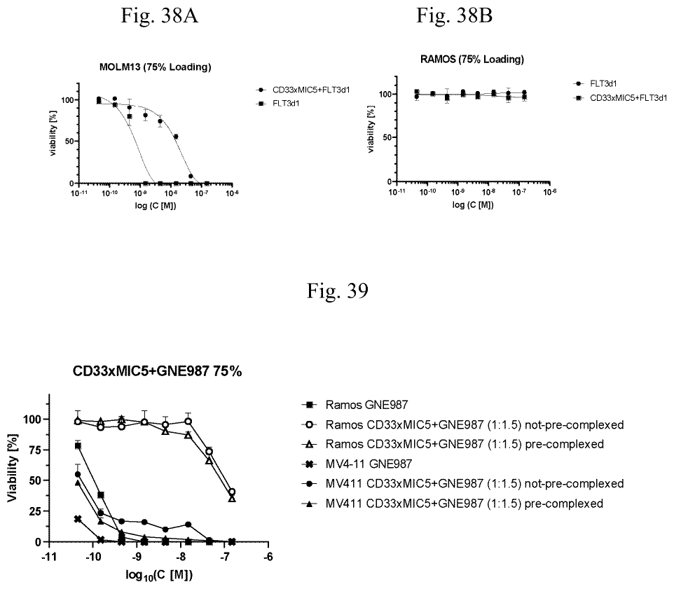

In order to expand the PAX approach to another intracellular target, it was investigated whether an FLT3 degrader can be delivered specifically to CD33-expressing cells using antibody-PROTAC complexes. Therefore, CD33-positive MOLM13 and CD33-negative RAMOS cells were treated with CD33xMIC5+FLT3d1 with 75% loading and PROTAC FLT3d1 alone, as control (FIGS. 38A-38B). On CD33-positive MOLM13 cells, cytotoxicity was observed for CD33xMIC5+FLT3d1. On CD33-receptor negative RAMOS cells no cytotoxicity was observed. This demonstrated that CD33xMIC5 might be used to deliver FLT3 degraders to CD33-positive cells. Assays were performed following the procedure described in chapter 7.10.5 with the slight modification that cells were treated with the PROTAC or PAX for 6 days. The experiment demonstrated again that PAX are able to deliver PROTACs to target cells depending on the receptor status. Additionally, the experiments demonstrated that the PAX technology can be transferred to another PROTAC degrading FLT3 to further underpin the versatility of the PAX approach.

### Complexation of PROTAC GNE987 by CD33xMIC5 can be Accomplished by Separate Application of Antibody and PROTAC on Cells

In another experiment, it was investigated if pre-complexing of CD33xMIC5 with the PROTAC GNE987 is necessary for cell-target receptor dependent cell killing. Therefore, CD33xMIC5 was complexed with GNE987 for 3 hours to reach a loading of 75% and added to CD33-positive MV411 and CD33-negative RAMOS cells. Additionally, CD33xMIC5 was added to the aforementioned cells and GNE987 was added subsequently and separately so that a loading of 75% was reached. Interestingly, the separate addition of CD33xMIC5 and GNE987 yielded the same results as the pre-complexed CD33xMIC5+GNE987 PAX with the same loading (FIG. 39).

These results demonstrated that pre-complexing of PAX is not a prerequisite for cell surface receptor dependent cell killing via PAX.

### Protac Loading of PAX can be Increased by Increasing the Number of Fused VHH Protac Binders to the Targeting Antibody

To increase the number of PROTACs that can be delivered into target cells by a single PAX molecule, the number of fused PROTAC-binding VHH units attached to the cell-targeting IgG was increased. In more detail, the MIC7 PROTAC-binding VHH was fused to the C-terminus of the CD33-targeting antibody on either the heavy chain, light chain or to a combination of both in different numbers. The antibody fragment was separated from the IgG heavy chain and from any additional fragment via a short linker. For further details see chapter 7.9. Then it was investigated how genetical fusions of the VHH fragments to different sites of the targeting-antibody effect the potency of the resultant PAX.

VHH-antibody fusions complexed with GNE987 or GNE987P in the complexation ratio depicted in Table 13 were investigated on CD33-positive MV411 and CD33-negative RAMOS cells according to the cell viability assay procedure described in 7.10.5. In case of combination with GNE987P the same antibody was loaded with different ratios of GNE987P and hence the treatment concentration was related to the antibody concentration. All fusions showed cell surface receptor-dependent cytotoxicity. Some VHH-antibody fusions loaded with GNE987P even showed enhanced potency on positive MV411 cells and reduced cytotoxicity on receptor negative RAMOS cells compared to the PROTAC alone. This further demonstrated, the versatility of this approach to generate multiple PAX with sophisticated properties.

TABLE 13
Cellular profiling of different CD33xMIC7 combined
with PROTACs GNE987 and GNE987P or PROTACs alone
on CD33-positive MV411 cells and CD33-negative
RAMOS cells. IC50 values are depicted in M.

| | | | IC50 | |
|---|---|---|---|---|
| Antibody | PROTAC | Ratio | MV411 | RAMOS |
| CD33xMIC7 | GNE987P | 1:2 | 4.7E−11 | 2.1E−08 |
| CD33xMIC7_{4H} | GNE987P | 1:4 | <3.0E−11 | >1.0E−07 |
| CD33xMIC7_{6H} | GNE987P | 1:6 | 3.7E−11 | 1.3E−08 |
| CD33xMIC7_{2L} | GNE987P | 1:2 | 9.3E−11 | 5.3E−08 |
| CD33xMIC7_{4L} | GNE987P | 1:4 | 8.9E−11 | >1.0E−07 |
| CD33xMIC7_{6L} | GNE987P | 1:6 | <3.0E−11 | 2.2E−08 |
| CD33xMIC7_{2H2L} | GNE987P | 1:4 | 8.7E−11 | 3.9E−08 |
| CD33xMIC7_{4H4L} | GNE987P | 1:8 | <3.0E−11 | 3.2E−08 |
| CD33xMIC7_{6H6L} | GNE987P | 1:12 | <3.0E−11 | 1.0E−08 |
| — | GNE987P | — | 5.5E−10 | 8.3E−09 |
| CD33xMIC7 | GNE987 | 1:1.8 | <5.4E−11 | 3.0E−10 |
| CD33xMIC7_{4H} | GNE987 | 1:1.8 | 3.4E−10 | >1.8E−07 |
| CD33xMIC7_{6H} | GNE987 | 1:1.8 | 1.1E−09 | >1.8E−07 |
| CD33xMIC7_{2L} | GNE987 | 1:1.8 | <5.4E−11 | 3.8E−10 |
| CD33xMIC7_{4L} | GNE987 | 1:1.8 | 2.0E−09 | >1.8E−07 |
| CD33xMIC7_{6L} | GNE987 | 1:1.8 | 3.9E−10 | >1.8E−07 |
| CD33xMIC7_{2H2L} | GNE987 | 1:1.8 | 2.7E−10 | >1.8E−07 |
| CD33xMIC7_{4H4L} | GNE987 | 1:1.8 | 4.4E−10 | >1.8E−07 |
| CD33xMIC7_{6H6L} | GNE987 | 1:1.8 | 1.4E−09 | >1.8E−07 |
| — | GNE987 | — | <4.5E−11 | 5.8E−11 |

### CLL1xMIC7-PROTAC Complexes Induce Selective Cytotoxicity Dependent on CLL1-Mediated Uptake

The scope of this invention was so far limited to CD33-targeting antibodies and therefore it was investigated if it is possible to deliver PROTACs in a cell-selective manner using other antibodies aside CD33-targeting antibodies.

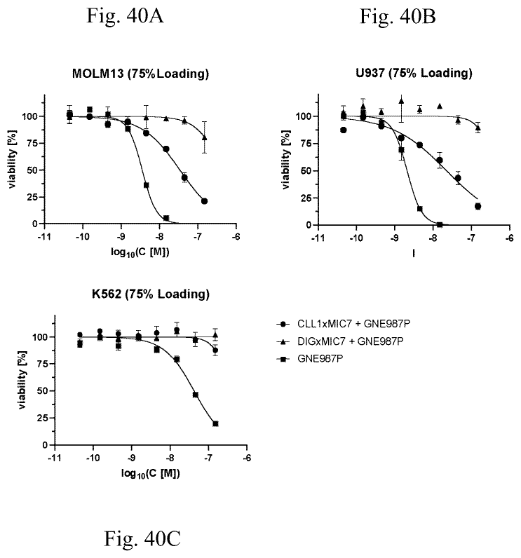

In order to target PROTACs to cells expressing CLL1, a fusion protein was constructed using the CLL1-binding antibody and the PROTAC-binding clone MIC7. Therefore, the HC of CLL1-binding antibody was elongated c-terminally by a linker followed by the sequence of MIC7 yielding CLL1xMIC7. Additionally, a digoxigenin antibody was modified in the same way to obtain an isotype control. CLL1-positive MOLM13 and U937 cells as well as CLL1-negative K562 cells were treated with CLL1xMIC7+GNE987P or DIGxMIC7+GNE987P with 75% loading or PROTAC GNE987P alone, as control. Assays were performed following the procedure described above. On both CLL1-positive cell lines, cytotoxicity was observed for CLL1xMIC7+GNE987P while no cytotoxicity was observed for DIGxMIC7+GNE987P (FIGS. 40A-C). This demonstrated again that uptake in receptor-positive cells is mediated by targeting the PAX to the desired cells. Furthermore, this demonstrated that this technology could be applied to different antibody backbones underlining the versatility of this approach.

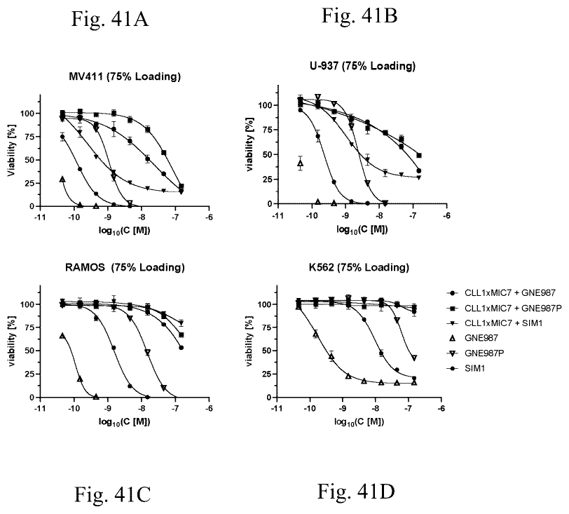

In an additional experiment, CLL1-positive MV411 and U937 or CLL1-negative RAMOS and K562 cells were treated with CLL1xMIC7+GNE987, CLL1xMIC7+GNE987P or CLL1xMIC7+SIM1 with 75% loading or GNE987, GNE987P or SIM1 alone, as controls. Assays were performed following the procedure described above. On both CLL1-positive cell lines, cytotoxicity was observed for CLL1xMIC7 PROTAC combinations while significantly less to no cytotoxicity was observed on CLL1-negative K562 and RAMOS cells (FIGS. 41A-41D). This demonstrated again that uptake in receptor-positive cells is mediated by targeting the PAX to the desired cells. Furthermore, this demonstrated that this technology can be applied to different antibody backbones that underpinned the versatility of this invention. Additionally, it was demonstrated that the choice of PROTAC is very flexible since a total of 3 different PROTACs were delivered to CLL1-expression cells using the PAX approach.

### B7H3xMIC7-PROTAC Complexes Induce Selective Cytotoxicity Dependent on B7H3-Mediated Uptake

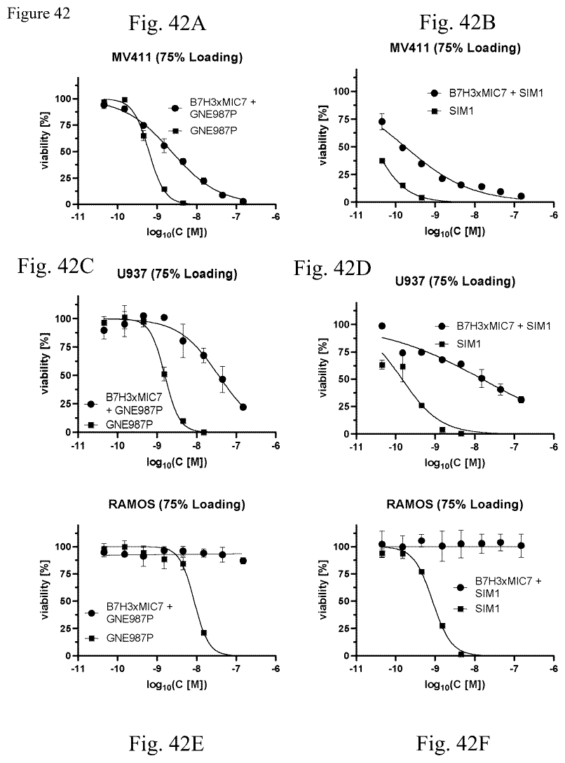

To further broaden the scope of this invention, it was investigated whether it is possible to deliver PROTACs in a cell-selective manner using B7H3-targeting antibodies. In order to target PROTACs to cells expressing B7H3, a fusion protein was constructed using a B7H3-binding antibody and the PROTAC-binding clone MIC7. Therefore, the HC of B7H3-binding antibody was elongated c-terminally by a linker followed by the sequence of MIC7 yielding B7H3xMIC7. B7H3-positive MV411, U937 and MOLM13 as well as B7H3-negative RAMOS cells were treated with B7H3xMIC7+GNE987P and B7H3xMIC7+SIM1 with 75% loading and PROTACs GNE987P and SIM1 alone, as controls. Assays were performed following the procedure described above. On all B7H3-positive cell lines, cytotoxicity was observed for all B7H3xMIC7 PROTAC combinations (FIGS. 42A-42F). On B7H3-receptor negative RAMOS cells no cytotoxicity was found for all B7H3xMIC7 PROTAC combinations, whereas treatment with PROTACs alone resulted in the highest, but not cell-type specific cytotoxicity. This demonstrated that B7H3xMIC7 mediated the uptake of GNE987P and SIM1 into receptor-positive cells while its uptake into receptor-negative cells was prevented. The experiment demonstrated again that PAX are able to deliver PROTACs to target cells depending on the receptor status. Additionally, the experiments demonstrated that PAX technology could be transferred to another antibody backbone to further underline the versatility of this approach.

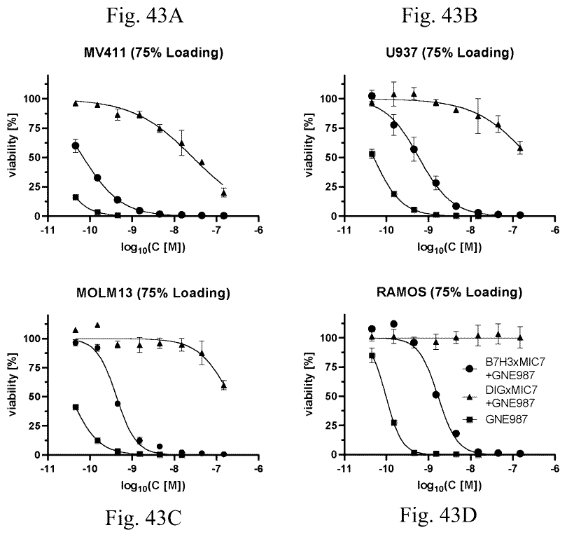

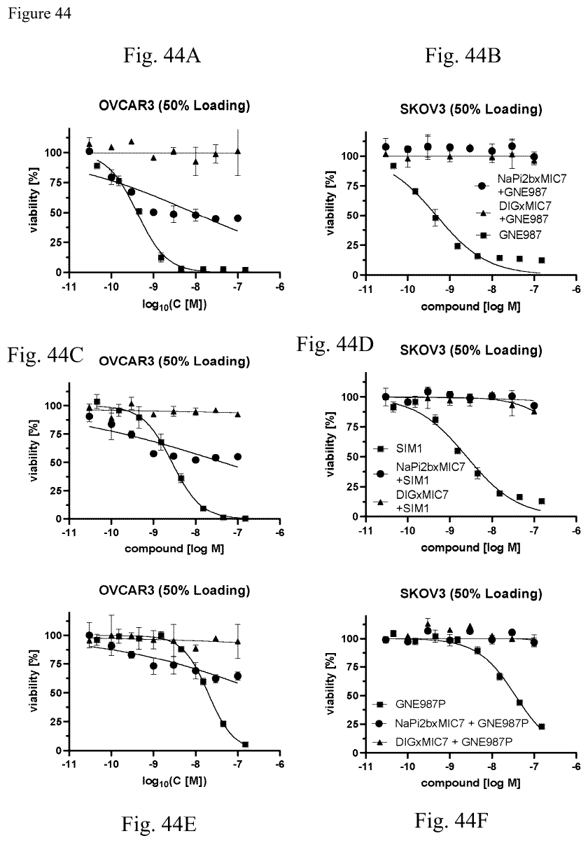

In another experiment, B7H3-positive MV411, U937 and MOLM13 as well as B7H3-negative RAMOS cells were treated with B7H3xMIC7+GNE987 or DIGxMIC7+GNE987 with 75% loading and PROTAC GNE987 alone, as control. Assays were performed following the procedure described above. On all B7H3-positive cell lines, cytotoxicity was observed for all B7H3xMIC7+GNE987 constructs whereas significantly less toxicity was found for isotype control DIGxMIC7+GNE987 (FIGS. 43A-43D). On B7H3-receptor negative RAMOS cells less cytotoxicity was found for B7H3xMIC7+GNE987 compared to the PROTAC alone. This further demonstrated that B7H3xMIC7 mediated the uptake of the GNE987P and SIM1 PROTAC into receptor-positive cells while it prevents uptake into receptor-negative cells.

### EGFRxMIC7-PROTAC Complexes Induce PROTAC-Loading Dependent and EGFR-Mediated Cell-Type Selective Cytotoxicity

In order to target PROTACs to cells expressing EGFR, a fusion protein was constructed using the EGFR-binding antibody Cetuximab and the PROTAC-binding clone MIC7. Therefore, the HC of Cetuximab was elongated c-terminally by a linker followed by the sequence of MIC7 yielding EGFRxMIC7. Additionally, a digoxigenin antibody was modified in the same way to obtain an isotype control. EGFR-high expressing OVCAR3 and SKOV3 as well as EGFR-low expressing HEPG2 were treated with EGFRxMIC7 and DIGxMIC7 loaded with 50% PROTAC, GNE987, GNE987P or SIM1 as described above to determine potency of those constructs (Table 14). Potencies of PROTACs alone are summarized in Table 14. While all DIGxMIC7+PROTAC combinations had an IC50>100 nM, EGFRxMIC7 combined with PROTACs GNE987 and SIM1 induced cell killing with IC50 values in single-digit nM range. The toxicity of EGFRxMIC7 combined with all PROTACs on EGFR-low expressing HEPG2 was reduced for all constructs (double- to triple-digit nM range) compared to EGFR-high expressing OVCAR3 and SKOV3. Overall, GNE987P combinations were inactive. The experiment demonstrated again that PAX are able to deliver PROTACs to target cells depending on the receptor status.

TABLE 14
Cellular profiling of PROTACs ARV771, GNE987,
GNE987P and EGFR-positive cells and EGFR-negative
HEPG2 cells. IC50 values are depicted in M.

| Cell line | ARV771 | GNE987 | GNE987P | SIM1 |
|---|---|---|---|---|
| A431 | 6.5E−08 | 3.4E−10 | 5.05−08 | 3.0E−09 |
| A549 | 1.5E−07 | 1.4E−09 | 1.5E−07 | 1.7E−08 |
| HCC827 | 9.1E−08 | 7.7E−10 | 7.7E−08 | 6.5E−09 |
| HEPG2 | 8.9E−08 | 2.6E−09 | 1.2E−07 | 1.4E−08 |
| MCF7 | 2.6E−08 | 1.5E−10 | 1.5E−08 | 1.5E−09 |
| MDAMB468 | 4.9E−08 | 4.1E−10 | 5.08−08 | 7.1E−09 |
| OVCAR3 | — | 4.0E−10 | 2.08−08 | 2.8E−09 |
| SKOV3 | 3.2E−08 | 6.1E−10 | 3.5E−08 | 2.4E−09 |

TABLE 15
Cellular profiling of EGFRxMIC7 combined with PROTACs GNE987, GNE987P
and SIM1 at a loading of 50% on EGFR-positive cells and EGFR-negative
HEPG2 cells. As a non-internalizing control, a digoxigenin-binding DIGxMIC7
fusion protein was utilized. IC50 values are depicted in M.

| PROTAC and Cell | EGFRxMIC7 | | | DIGxMIC7 | | |
|---|---|---|---|---|---|---|
| line | GNE987 | GNE987P | SIM1 | GNE987 | GNE987P | SIM1 |
| HEPG2 | 4.1E−08 | >1.0E−07 | 2.0E−08 | >1.0E−07 | >1.0E−07 | >1.0E−07 |
| OVCAR3 | 4.8E−09 | >1.0E−07 | 2.0E−09 | >1.0E−07 | >1.0E−07 | >1.0E−07 |
| SKOV3 | 7.5E−09 | >1.0E−07 | 2.0E−09 | >1.0E−07 | >1.0E−07 | >1.0E−07 |

In another experiment, EGFRxMIC7 was loaded with the PROTACs ARV771, GNE987, GNE987P and SIM1 separately at a loading of 75% and a range of cells with varying EGFR expression levels were treated (Table 16). Overall, the non-binding control DIGxMIC7 loaded with 75% of GNE987, GNE987P and SIM1 was less potent than the EGFR-binding EGFRxMIC7 loaded with the same PROTACs.

TABLE 16
Cellular profiling of EGFRxMIC7 combined with PROTACs ARV771, GNE987, GNE987P and SIM1 at
a loading of 75% on EGFR-positive cells and EGFR-negative HEPG2 and EGFR-low MCF7 cells.
As a non-internalizing control, a digoxigenin-binding DIGxMIC7 fusion protein was utilized.

| PROTAC and cell | EGFRxMIC7 | | | | DIGxMIC7 | | | |
|---|---|---|---|---|---|---|---|---|
| line | ARV771 | GNE987 | GNE987P | SIM1 | ARV771 | GNE987 | GNE987P | SIM1 |
| A431 | 1.1E−07 | 1.2E−09 | 2.7E−09 | 2.3E−09 | 9.3E−08 | 3.5E−09 | >1.5E−07 | >1.5E−07 |
| A549 | >1.5E−07 | 3.3E−09 | >1.5E−07 | >1.5E−07 | >1.5E−07 | 1.8E−08 | >1.5E−07 | >1.5E−07 |
| HCC827 | >1.5E−07 | 8.3E−10 | 1.4E−07 | 2.0E−08 | >1.5E−07 | 7.6E−08 | >1.5E−07 | >1.5E−07 |
| HEPG2 | >1.5E−07 | 4.0E−09 | >1.5E−07 | >1.5E−07 | 1.1E−07 | 2.4E−08 | >1.5E−07 | >1.5E−07 |
| MCF7 | 4.8E−08 | 6.5E−10 | 2.6E−08 | 1.1E−08 | 3.0E−08 | 1.8E−09 | >1.5E−07 | >1.5E−07 |
| MDAMB468 | 6.3E−08 | 4.7E−10 | 1.3E−08 | 6.8E−09 | 4.8E−08 | 2.7E−09 | >1.5E−07 | >1.5E−07 |
| SKOV3 | 7.4E−08 | 1.6E−09 | 9.2E−08 | 5.4E−08 | 5.1E−08 | 5.1E−09 | >1.5E−07 | >1.5E−07 |

In conclusion, it was demonstrated that PAX can also target PROTACs to solid tumor cells. It was also shown that the uptake is dependent on the EGFR-receptor expression levels and therefore driven by active uptake through binding of EGFR by the EGFRxMIC7+PROTAC complexes followed by internalization and PROTAC release. Cell-selectivity driven by active EGFR-mediated uptake was also shown by testing a non-binding isotype control PAX which showed significantly less cytotoxicity.

NAPI2BxMIC7 Complexes with GNE987, GNE987P and SIM1 Exhibit Cell-Selective Cytotoxicity

In order to target PROTACs to cells expressing NAPI2B, a fusion protein was constructed using the NAPI2B-binding antibody XMT1535 and the PROTAC-binding clone MIC7. Therefore, the HC of NAPI2B-binding antibody was elongated c-terminally by a linker followed by the sequence of MIC7 yielding NAPI2BxMIC7. NAPI2B-positive OVCAR3 and NAPI2B-negative SKOV3 cells were treated with NAPI2BxMIC7+GNE987, NAPI2BxMIC7+GNE987P, NAPI2BxMIC7+SIM1 or DIGxMIC7+GNE987, DIGxMIC7+GNE987P or DIGxMIC7+SIM1 with 50% loading and PROTACs GNE987, GNE987P and SIM1 alone, as controls. Assays were performed following the procedure described above. On NAPI2B-positive OVCAR3, cytotoxicity was observed for all NAPI2BxMIC7 PROTAC combinations (FIGS. 44A-44F). On NAPI2B-receptor negative SKOV3 cells no cytotoxicity was found for all NAPI2BxMIC7 PROTAC combinations, whereas treatment with PROTACs alone resulted in the highest observed cytotoxicity on all cell lines independent of the NAPI2B receptor expression status. This demonstrated that NAPI2BxMIC7 mediated the uptake of GNE987, GNE987P and SIM1 into receptor-positive cells while their uptake into receptor-negative cells was prevented. The experiment demonstrated again that PAX are able to deliver PROTACs to target cells depending on the receptor status. Additionally, the experiments demonstrated that PAX technology could be transferred to another antibody backbone to further underline the versatility of this approach.

HER2xMIC7 Combined with PROTACs GNE987, GNE987P and SIM1 Show HER2-Dependent Cytotoxicity

In order to target PROTACs to cells expressing HER2, a fusion protein was constructed using the HER2-binding antibody trastuzumab and the PROTAC-binding clone MIC7. Therefore, the HC of HER2-binding antibody was elongated c-terminally by a linker followed by the sequence of MIC7 yielding HER2xMIC7. HER2-positive SKBR3 and NCIN87 cells and HER2-negative MDAMB468 cells were treated with HER2xMIC7+GNE987, HER2BxMIC7+GNE987P, HER2xMIC7+SIM1 with 75% loading and PROTACs GNE987, GNE987P and SIM1 alone, as control. Assays were performed following the procedure described above. On HER2-positive SKBR3 and NCIN87, cytotoxicity was observed for all HER2xMIC7 PROTAC combinations (Table 17). On HER2-receptor negative MDAMB468 cells no cytotoxicity was found for all HER2xMIC7 PROTAC combinations, whereas treatment with the PROTACs alone resulted in pronounced cytotoxicity on all cell lines independent of the HER2 receptor expression status. This demonstrated that HER2xMIC7 mediated the uptake of GNE987, GNE987P and SIM1 into receptor-positive cells while their uptake into receptor-negative cells was prevented. The experiment demonstrated again that PAX are able to deliver PROTACs to target cells depending on the receptor expression status. Additionally, the experiments demonstrated that PAX technology could be transferred to another antibody backbone to further underline the versatility of this approach.

TABLE 17
Cellular profiling of HER2xMIC7 combined with the PROTAC GNE987, GNE987P and SIM1
at a loading of 75% on HER2-positive cells and HER2-negative MDAMB468 cells.

| | IC50 [M] of | IC50 [M] of | IC50 [M] of | IC50 [M] | IC50 [M] | |
|---|---|---|---|---|---|---|
| | HER2xMIC7 + | HER2xMIC7 + | HER2xMIC7 + | of | of | IC50 [M] |
| Cell line | GNE987 | GNE987P | SIM1 | GNE987 | GNE987P | of SIM1 |
| NCIN87 | 8.8E−09 | 3.2E−08 | 3.0E−08 | 5.3E−10 | 5.0E−08 | 4.0E−09 |
| SKBR3 | 2.7E−08 | 3.1E−08 | 1.4E−08 | 3.9E−10 | 2.7E−08 | 3.6E−09 |
| MDAMB468 | 1.5E−07 | 1.5E−07 | 1.5E−07 | 4.1E−10 | 5.0E−08 | 7.1E−09 |

### TROP2xMIC7+GNE987 Mediates TROP2-Dependent Cytotoxicity

To further broaden the scope of this invention, it was investigated whether it is possible to deliver PROTACs in a cell-selective manner using TROP2-targeting antibodies. In order to target PROTACs to cells expressing TROP2, a fusion protein was constructed using the TROP2-binding antibody sacituzumab and the PROTAC-binding clone MIC7. Therefore, the HC of TROP2-binding antibody was elongated c-terminally by a linker followed by the sequence of MIC7 yielding TROP2xMIC7. TROP2-positive A431, SKBR3, MDAMB468, NCIN87, SKOV3 cells and TROP2-negative SW620 cells were treated with TROP2xMIC7+GNE987 with 75% loading or PROTAC GNE987 alone, as control. Assays were performed following the procedure described in chapter 7.10.5. On all TROP2-positive A431, SKBR3, MDAMB468, NCIN87 and SKOV3 cells cytotoxicity was observed for TROP2xMIC7+GNE987 (Table 18). Reduced cytotoxicity was found on TROP2-negative SW620 cells for TROP2xMIC7+GNE987 compared to GNE987 alone. These results demonstrated that TROP2xMIC7 mediated the uptake of GNE987 into receptor-positive cells while their uptake into receptor-negative cells was reduced. The experiment demonstrated again that PAX are able to deliver PROTACs to target cells depending on the receptor expression status. Additionally, the experiments demonstrated that PAX technology could be transferred to another antibody backbone to further underline the versatility of this approach.

TABLE 18
Cellular profiling of TROP2xMIC7 combined with
the PROTAC GNE987 at a loading of 75% on TROP2-
positive cells and TROP2-negative SW620 cells.

| | IC50 of TROP2xMIC7 + | IC50 of |
|---|---|---|
| Cell line | GNE987 [M] | GNE987 [M] |
| A431 | 2.9E−09 | 3.46−10 |
| SKBR3 | 1.3E−08 | 3.9E−10 |
| MDAMB468 | 2.1E−08 | 4.1E−10 |
| NCIN87 | 4.1E−09 | 5.3E−10 |
| SKOV3 | 5.6E−08 | 6.1E−10 |
| SW620 | 7.1E−08 | 8.4E−10 |

### Comparable Cytotoxicity of PAX and PROTAC-ADCs

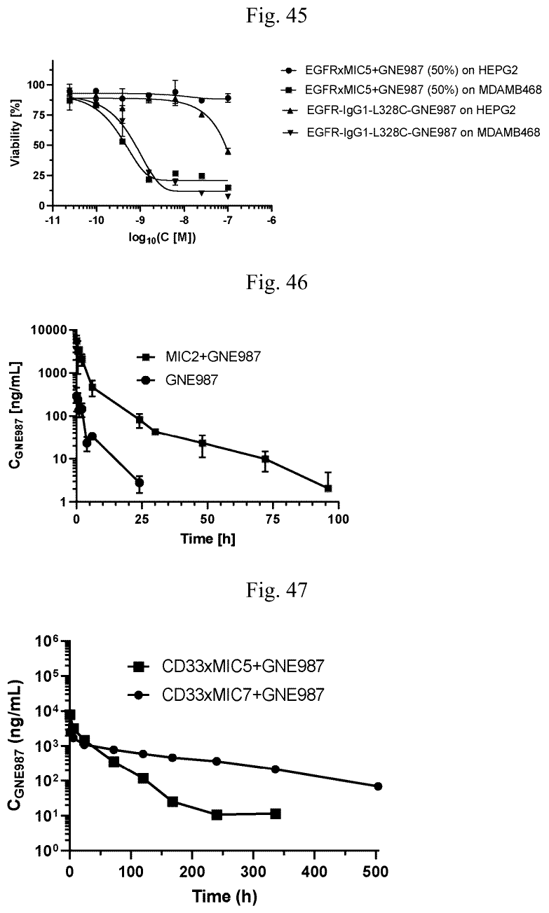

In order to understand how the PAX technology compares to covalently-linked PROTAC-ADCs, the EGFR-IgG1-L328C-GNE987 PROTAC-ADC (DAR 1.62) described in chapter 7.5.1 was investigated together with EGFRxMIC5 loaded with 50% GNE987 on EGFR-positive MDAMB468 and EGFR-negative HEPG2 cells. For better comparison the treatment concentration was related to the antibody concentration. It was observed, that both constructs had the same potency on MDAMB468 cells and showed fewer but comparable cytotoxic effects on HEPG2 cells (FIG. 45). This demonstrated that the PAX, although the PROTAC is only associated non-covalently, can enable selective cell-killing comparable to the effects of a covalent PROTAC-ADC. Further this effect can be achieved with a lower DAR compared to PROTAC-ADC.

The Complexation of PROTACs with Anti-PROTAC Antibodies can Significantly Improve Pharmacokinetic Profile

It was investigated if and to what extent the complexation of the PROTAC GNE987 with the PROTAC-binding antibody MIC2 influences the overall pharmacokinetic profile of the PROTAC. Therefore, a pharmacokinetic study was performed as follows:

Female SCID beige (n=9, composite profile) received a single tail vein intravenous (i.v.) bolus injection of PROTAC alone (GNE987) at 0.4 mg/kg in 2% (v/v) DMSO/20% (v/v) (hydroxypropyl β-cyclodextrin) Kleptose in water, at a dosing volume of 5 ml/kg. For MIC2+GNE987 (antibody: drug ratio of 1:2), female SCID beige (n=12, composite profile) received a single tail vein intravenous (i.v.) bolus injection of the PROTAC shuttle at an equivalent dose of 0.4 mg/kg of GNE987 and 30 mg/kg of MIC2 in 5% (v/v) DMSO in PBS, at a dosing volume of 5 ml/kg.

For the PROTAC alone, consecutive blood samples were taken (n=3) at 0.1 (G (group) 1): 0.5 (G2), 1 (G3), 2 (G1), 4 (G3), 6 (G2) and 24 h (G3) after i.v. administration, sub-lingually under isoflurane anesthesia and with ethylene diamine tetra-acetic acid (K3-EDTA) as anti-coagulant, and further processed to obtain plasma.

For MIC2+GNE987, consecutive blood samples were taken (n=3) at 0.1 (G (group) 1), 0.5 (G2), 1 (G3), 2 (G4), 6 (G2), 24 h (G1, G3), 30 (G3), 48 (G3, G4)), 72 (G1, G2) and 96 h (G2) after i.v. administration, as described above, and further processed to obtain plasma.

For the sample preparation, 10 μL plasma was diluted with 10 μL of methanol and precipitated with 80 μL of acetonitrile, containing Labetalol s internal standard (2.5 μg/mL), in LowBind (protein) plates. After shaking/vortexing for 1 min, samples were filtered, (Captiva filtration on polypropylene filter, 0.45 μm pore size) and 120 μL of methanol:water (1:1, v/v) was added to the filtrate and stored at 4° C. until analysis and put in the autosampler before injection. The analysis was carried out on a LC-MS/MS system consisting of an UPLC coupled to a QTRAP 6500+ (Sciex) mass spectrometer. Mobile phase A was water with 0.1% formic acid and mobile phase B was methanol with 0.1% formic acid. The gradient was started with 10% B to 95% B in 1.5 min and maintained at 95% B for 2 min, then decreased to 10% B in 0.5 min and maintained to 10% B for 2 min. The chromatography was performed on a Poroshell 120 EC-C18 column, 2.7 μm particles, 3×50 mm, from Agilent Technologies. The flow rate was 0.6 mL/min and the cycle time (injection to injection) was approximately 6 minutes The sample injection volume was 10 μL. MRM transition for GNE987 was 548.788 (m/z, z=2)→779.2 (m/z, z=1) and 329.101 (m/z, z=1)→91 (m/z, z=1) for labetalol (IS). The calibration curve for quantitation was based on standards ranging from 0.5 (Lower Limit of Quantitation) to 10000 (Upper Limit of Quantitation) ng/ml, with 5 calibration points minimum and minimum 75% of calibration standards to be within #20% of their nominal values.

The total antibody concentration was determined by ligand binding assay (LBA) based on the Meso Scale Diagnostics technology (MSD, LLC., Rockville, MD). All incubation steps were performed at 22° C. with gentle agitation. All washing steps (200 μL/well) were performed with PBS-T, containing PBS pH 7.4 and 0.01% Tween 20, using the plate washer ELx405 (BioTek instruments Inc., Winooski, VT). First, 2.5 μg/mL biotin-SP-conjugated AffiniPure goat anti-human IgG, Foy fragment specific (Jackson ImmunoResearch Europe Ltd., JIR, Cambridgeshire, United Kingdom, #109-065-098) was coated on MSD GOLD 96-well Streptavidin QUICKPLEX Plates (MSD, #L55SA) for 2 h. Afterwards, plates were washed three times. Plasma samples, standards and quality controls were serially diluted in dilution buffer, consisting of PBS pH 7.4, 0.05% Tween 20 and 3.0% (w/v) BSA, and incubated on the plates for 1 h. The plates were washed again and incubated for 1 h with 0.6 μg/mL mouse anti-human IgG, F(ab′)2 fragment specific (JIR, #209-005-097), previously labeled with MSD GOLG SULFO-TAG (MSD, #R31AA-1) according to the manufacturing procedure. After a final washing step, 150 μL of 2×MSD Read Buffer T with surfactant (MSD, #R92TC) was added to each well and plates were read on a MESO Quickplex SQ120 plate reader (MSD). The Software Watson LIMS (Version 7.5, ThermoFisher Scientific Inc.) was used to fit the standard curve with a 5PL (Marquart) equations, weighting factor 1/Y2, and to calculate the total mAb concentration of the plasma samples. The lower limit of quantification (LLOQ) was 50 ng/ml. The half-life of the PROTAC GNE987 was determined to be 5.8 hours which is in the same range as the half-life reported in literature (2.8 hours, Pillow, T. H. et al., ChemMedChem 15 (2020) 17-25). The complexation of the PROTAC GNE987 by MIC2 led to a half-life of PROTAC GNE987 of 14.7 h in mice, which corresponds to a 2.5-fold half-life improvement.

Additionally, the PAX CD33xMIC5+GNE987 and CD33xMIC7+GNE987 were generated with a theoretical loading of 100% and investigated in PK studies conducted in C57BL/6N inbred mice (N=2 males and females for each group) provided by Charles River Laboratories Italia, Calco, Italy. The 7-8-week-old mice received 30 mg/kg (corresponding to 0.38 mg/kg PROTAC) of CD33xMIC5+GNE987 and CD33xMIC7+GNE987, CD33 antibody alone, CD33xMIC5 or CD33xMIC7 as single dose, that was intravenously injected into the tail vein. Samples have been serially collected from all animals using a microsampling technique (20 mL for each blood withdrawal). After administration, two blood samples were taken on the first day and 7 during the three weeks later. Each sample was collected in pre-chilled (0-4° C.) Minivette POCT EDTA tube, transferred in Microvette CB300 EDTA and centrifuged at 2500×g for 10 min at 4° C. The obtained plasma was transferred into a new vial and immediately stored at −80° C. until further analyses. The PK study, animal handling and experimentation, was conducted in accordance with the Italian D.Lvo. 2014/26 and Directive 2010/63/EU. The study was performed at the Instituto di Ricerche Biomediche Antoine Marxer, Colleretto Giacosa, Italy. The institute is fully authorized by the Italian Ministry of Health.

The total antibody concentration was determined by ligand binding assay (LBA) based on the Meso Scale Diagnostics technology (MSD, LLC., Rockville, MD). All incubation steps were performed at 22° C. with gentle agitation. All washing steps (200 μL/well) were performed with PBS-T, containing PBS pH 7.4 and 0.01% Tween 20, using the plate washer ELx405 (BioTek instruments Inc., Winooski, VT). First, 2.5 μg/mL biotin-SP-conjugated AffiniPure goat anti-human IgG, Fcγ fragment specific (Jackson ImmunoResearch Europe Ltd., JIR, Cambridgeshire, United Kingdom, #109-065-098) was coated on MSD GOLD 96-well Streptavidin QUICKPLEX Plates (MSD, #L55SA) for 2 h. Afterwards, plates were washed three times. Plasma samples, standards and quality controls were serially diluted in dilution buffer, consisting of PBS pH 7.4, 0.05% Tween 20 and 3.0% (w/v) BSA, and incubated on the plates for 1 h. The plates were washed again and incubated for 1 h with 0.6 μg/ml mouse anti-human IgG, F(ab′)2 fragment specific (JIR, #209-005-097), previously labeled with MSD GOLG SULFO-TAG (MSD, #R31AA-1) according to the manufacturing procedure. After a final washing step, 150 μL of 2×MSD Read Buffer T with surfactant (MSD, #R92TC) was added to each well and plates were read on a MESO Quickplex SQ120 plate reader (MSD). The Software Watson LIMS (Version 7.5, ThermoFisher Scientific Inc.) was used to fit the standard curve with a 5PL (Marquart) equations, weighting factor 1/Y2, and to calculate the total mAb concentration of the plasma samples. The lower limit of quantification (LLOQ) was 50 ng/ml. The concentration of MSC2734242 was determined by liquid chromatography tandem mass spectrometry (LC-MS/MS) using a SCIEX 5500 triple quadrupole with Turbo Ion Spray source (ITS) in positive modality (SCIEX, Redwood City, CA, USA). Chromatographic separation was achieved using a Waters ACQUITY UPLC BEH (C18, 2.1×50 mm, 1.7 μm) column, mounted in a Waters ACQUITY I-class UPLC system (Milford, MA, USA), configured with a 100 μL extension loop. Chromatographic gradient used for phase A (H₂O:ACN 95:5, 0.1% Formic Acid) and B (ACN:H2O 95:5, 0.1% Formic Acid) at flow 0.350 mL/min, was 0.25 min of 100% A isocratic, followed by a 2.25 min gradient to 100% B, with a subsequent 0.75 min of washing step at 100% B and 2.5 min of reconditioning at initial conditions.

Extraction of MSC2734242 from C57BL/6N mouse plasma samples was carried out by protein precipitation technique. 3 μL of plasma sample were precipitated in 100 μL of acetonitrile containing 50 ng/ml of MSC2737500, used as internal standard, on a Phenomenex Impact Protein Precipitation Plate (Phenomenex, Torrance, CA, USA, CE0-7565). After 5 min shaking (900 rpm) all the wells were filtered by vacuum and collected in a clean 96 wells plate, then diluted with 100 μL of an aqueous solution containing 2.5% Formic Acid and submitted to LC-MS/MS analysis. All reagents were LC-MS grade or equivalent.

The Software Watson LIMS (Version 7.5, ThermoFisher Scientific Inc.) was used to fit the standard curve on the area ratio (analyte signal/internal standard signal) on a linear regression, weighting factor 1/X2, and to calculate the total MSC2734242 concentration of the plasma samples. The lower limit of quantification (LLOQ) was 5 ng/mL, and the complete range of quantitation was 5-2000 ng/ml.

Main PK parameters were estimated by noncompartmental analysis (NCA) using Phoenix WinNonlin version 8.3.4 (Pharsight Corporation, USA). The pharmacokinetic parameters have been obtained or calculated from the individual plasma concentrations of total antibody and MSC2734242 analyte vs. time after administration.

Individual plasma concentration-time profiles were used for parameter estimation. The concentration of all PK samples that were calculated below quantification limit (BQL) were considered as missing value to better estimate AUCO-inf, Clearance and Volume of distribution. The terminal half-life (t½) and λz (the first order rate constant associated with the terminal log-linear portion of the curve) values have been calculated only when at least three time points were quantifiable in the terminal phase of the linear regression. Values below the BQL were considered 0 ng/ml for descriptive statistics. Overall, the complexation of GNE987 with either CD33xMIC5 and CD33xMIC7 led to a significant longer half-life of the PROTAC in the mice with 29.6 h and 105 h, respectively (FIG. 47). Impressively, the concentration of GNE987 was still 69 ng/ml on average in the mouse plasma even after 21 d (504 h) in the group that received CD33xMIC7+GNE987 while the PROTAC GNE987 concentration fell already below LLOQ after 25 h. The results illustrate that antibody-complexation might strongly increase exposure of, e.g., tumor cells to PROTACs by complexation of the PROTAC with antibody.

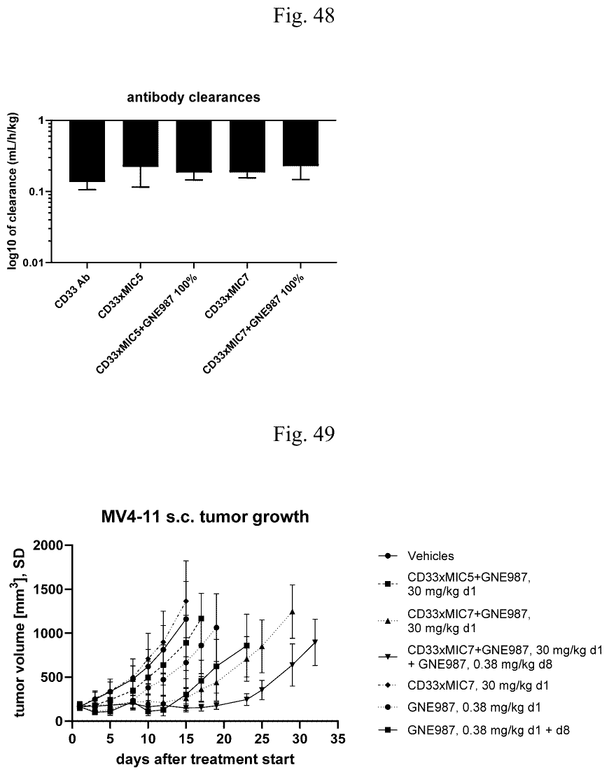

The antibody clearances of the PK study were compared to draw conclusions if the generation of VHH fusions and the loading of the respective fusion proteins with PROTAC GNE987 did alter the clearance rate (FIG. 48). The clearances of the unmodified CD33 antibody and the fusions of the same antibody with the VHL-PROTAC binding VHHs MIC5 and MIC7 (CD33xMIC5/7) were similar which suggests are minor impact of VHH fusion on the clearance. Furthermore, the loading of the antibody-VHH fusion proteins CD33xMIC5 and CD33xMIC7 with the PROTAC GNE987 had no impact on the antibody clearance. Concluding, the addition of the VHL-PROTAC binding VHH and the complexation with PROTAC had no impact on the PK profile.

The Pharmacokinetic parameters of the latter study are summarized in Table 19.

TABLE 19
Summary of pharmacokinetic parameter of CD33-based VHH-fusions with MIC5 and
MIC7 loaded and unloaded with PROTAC GNE987 and the parental antibody CD33
Ab. The analytes were administered at 30 mg/kg and the PK parameters for the
quantification total antibody (tAntibody) and the PROTAC GNE987 are depicted.

| | Dose_ | | | t½ | Cmax | AUC0-inf | Cl | Vss |
|---|---|---|---|---|---|---|---|---|
| ID | (mg/kg) | Analyte | Subject | (h) | (ng/mL) | (h*ng/mL) | (mL/h/kg) | (mL/kg) |
| CD33 Ab | 30.00 | tAntibody | Mean | 237.0 | 810000 | 219000000 | 0.140 | 74.2 |
| | | | SD | 3.5 | 138000 | 40000000 | 0.0 | 26.3 |
| CD33xMIC5 | 30.00 | tAntibody | Mean | 351.0 | 661000 | 171000000 | 0.217 | 85.1 |
| | | | SD | 214.0 | 107000 | 93000000 | 0.104 | 8.6 |
| CD33xMIC5 + GNE987 | 30.00 | GNE987 | Mean | 29.6 | 7930 | 126000 | 3.0 | 99.8 |
| | | | SD | — | — | — | — | — |
| | 30.00 | tAntibody | Mean | 276.0 | 717000 | 170000000 | 0.184 | 66.8 |
| | | | SD | 139.0 | 190000 | 43000000 | 0.0 | 9.9 |
| CD33xMIC7 | 30.00 | tAntibody | Mean | 274.0 | 679000 | 164000000 | 0.186 | 74.8 |
| | | | SD | 29.3 | 179000 | 24100000 | 0.0 | 7.2 |
| CD33xMIC7 + GNE987 | 30.00 | GNE987 | Mean | — | 119 | — | — | — |
| | | | SD | — | — | — | — | — |
| | 30.00 | tAntibody | Mean | 268.0 | 568000 | 135000000 | 0.236 | 86.1 |
| | | | SD | 62.6 | 72400 | 38100000 | 0.1 | 10.6 |
| CD33xMIC7 + GNE987 | 30.00 | GNE987 | Mean | 105.0 | 2520 | 231000 | 1.7 | 270.0 |
| | | | SD | 24.2 | 138 | 65200 | 0.526 | 12.8 |
| | 30.00 | tAntibody | Mean | 304.0 | 591000 | 132000000 | 0.0 | 0.0 |
| | | | SD | 58.6 | 144000 | 43500000 | 0.0 | 0.0 |

Abbreviations: t_{1/2}: half-life; Cmax: maximum serum concentration; AUC0-inf: Area under the curve to infinite time; Cl: Clearance; Vss: Steady state volume of distribution.
SD: Standard deviation.

### CD33xMIC7+GNE987 PROTAC-Antibody Complexes are Efficacious in MV411 Mouse Xenograft Model

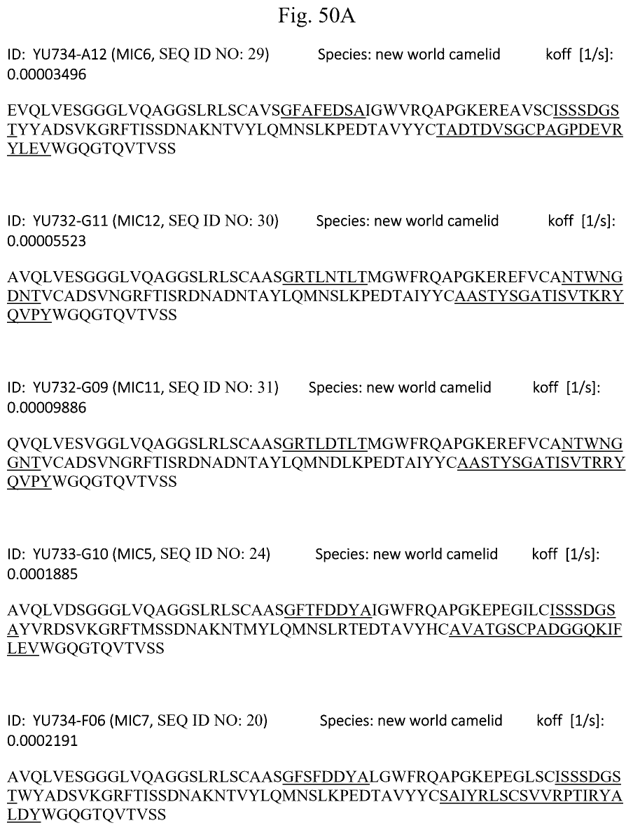

Human leukemic MV411 cells were xenografted in immunocompromised mice. Three million MV411 cells were injected subcutaneously in the left flank of untreated female CB17 SCID mice. Randomization of the animals in the different treatment groups and the initiation of the treatment was started after the average tumor size reached 45 mm². The control groups were treated with vehicle. The test groups were treated with 0.38 mg/kg GNE987 and 30 mg/kg CD33xMIC7+GNE987 (loaded with 0.38 mg/kg GNE987) once (at day 1). Further, two groups were treated twice, either with 30 mg/kg CD33xMIC7+GNE987 (loaded with 0.38 mg/kg GNE987) at day 1 followed by treatment with 0.38 mg/kg GNE987 at day 8 or with 0.38 mg/kg GNE987 at day 1 and 8. Additionally, one group received 30 mg/kg CD33xMIC5+GNE987 (loaded with 0.38 mg/kg GNE987) once (at day 1) and another group received the antibody control 30 mg/kg CD33xMIC7 without PROTAC once (at day 1). The individual groups were stopped before tumors reached a maximum tumor size (225 mm²) (FIG. 49).

While the antibody alone had no significant relevant effect over the vehicle control, the PROTAC GNE987 induced anti-tumor effects. However, at ˜day 4 the tumors started to progress again, showing the same growth rate as the vehicle control. In the group with dosing GNE987 at day 1 and 8 the re-dosing of GNE987 again induced anti-proliferative effects until ˜day 3 post re-dosing where tumors started to grow again. A single dose of CD33xMIC7+GNE987 led to tumor-growth inhibition until day 15 after which the tumors progressed. In the group dosed with 30 mg/kg CD33xMIC7+GNE987 (loaded with 0.38 mg/kg GNE987) at day 1 followed by treatment with 0.38 mg/kg GNE987 at day 8 the re-dosing led to sustained tumor growth inhibition until ˜day 23. The treatment with CD33xMIC5+GNE987 induced tumor-growth delay compared to vehicle but the effect was much less pronounced compared to CD33xMIC7+GNE987.

Overall, the anti-tumor effects of CD33xMIC7 loaded with GNE987 were superior to PROTAC alone at an equivalent PROTAC dose. Interestingly, the anti-tumor effects of CD33xMIC7+GNE987 could even be enhanced through simply re-dosing of GNE987 at day 8. This demonstrates that there is a clear benefit of additional dosing of GNE987 alone to a group that received CD33xMIC7+GNE987 and it is likely that CD33xMIC7 captures the PROTAC GNE987 from the serum and accumulates it at the tumor site. These results open avenue for pretargeting the tumor by administration of a bispecific antibody, that binds to a tumor specific antigen as well as to a PROTAC, followed by administration of an uncomplexed PROTAC (e.g., an orally applicable one). With this approach the separately administered PROTAC can be targeted to a desired tissue without the need of manufacturing the PAX ex vivo before.

When comparing the anti-PROTAC clones MIC5 and MIC7, it becomes apparent that CD33xMIC7+GNE987 induces stronger anti-tumor effects than CD33xMIC5+GNE987. The binding of CD33 did not impact tumor-growth as demonstrated by treatment with CD33xMIC7 which underpins that the anti-tumor effects are driven by loading CD33xMIC7 with PROTAC GNE987.

### SUMMARY

The present invention discloses an unprecedented delivery technology that enables targeted delivery of PROTACs via non-covalent PROTAC antibody complexes (PAX). It is important to note that the PAX technology allows the targeted delivery of unmodified active PROTACs unlike other methods in the field of non-covalent drug delivery where the active drug substance is usually modified with a hapten. This invention encompasses the delivery of PROTACs to multiple cell types depending on their cell surface receptor expression. Those receptors include but are not limited to: CD33, CLL1, TROP2, HER2, EGFR, NAPI2B and B7H3. With regard to PROTACs, the versatility of the invention is demonstrated by the selective delivery of a variety of structurally different PROTACs (GNE987, ARV771, SIM1, GNE987P, SIM1 and FLT3d1). It has also been demonstrated that the platform offers the possibility to delivery up to 12 PROTAC molecules using one antibody by leveraging modular antibody-engineering strategies. Moreover, the invention demonstrates that antibody-complexation can significantly improve a target cell's exposure to the PROTAC. Lastly, the inventors were able to validate the PAX technology in an in vivo xenograft model. Table 20 gives an overview.

TABLE 20
Summary of the scope of this work. The investigated
combinations are depicted in tabular form.

| Antibody | Clone | PROTAC | Loading |
|---|---|---|---|
| CD33 | MIC5-MIC8, | GNE987, ARV771, | Up to 12 |
| | MIC11-MIC14 | GNE987P, SIM1, FLT3d1 | |
| CLL1 | MIC7 | GNE987, GNE987P, SIM1 | Up to 2 |
| TROP2 | MIC7 | GNE987 | Up to 2 |
| HER2 | MIC7 | GNE987, GNE987P, SIM1 | Up to 2 |
| EGFR | MIC5-MIC14 | ARV771, GNE987, | Up to 2 |
| | | GNE987P, SIM1 | |
| NAPI2B | MIC7 | GNE987, GNE987P, SIM1 | Up to 2 |
| B7H3 | MIC7 | GNE987, GNE987P, SIM1 | Up to 2 |

1. An isolated antibody, capable of binding to the VHL ligand degron of a PROTAC.

2. The antibody of claim 1 which is a monospecific antibody.

3. The antibody of claim 1 or 2, which is a full-length antibody of the IgG type, or a fragment thereof, or a single domain antibody, or a single chain antibody.

4. The antibody of claim 3, wherein the full-length antibody is of the IgG1 or IgG4 type.

5. The antibody of claim 3, wherein the single domain antibody is a VHH antibody.

6. The antibody of claim 3, wherein the single chain antibody is a monospecific monovalent chain single antibody (scFv).

7. The antibody of any of claim 1, or 3-6, which is a bi-specific antibody, wherein the second binding is for a capability target protein.

8. The antibody of claim 7, comprising
a) a monospecific bivalent antibody consisting of two full length antibody heavy chains and two full length antibody light chains, wherein each chain comprises only one variable domain,
b) two monospecific monovalent single chain antibodies (scFv's), each consisting of an antibody heavy chain variable domain, an antibody light chain variable domain, and a single-chain-linker between said antibody heavy chain variable domain and said antibody light chain variable domain, and, optionally,
c) peptide-linkers, connecting the C-termini of part (a) and the N-termini of part (b).

9. The antibody of claim 7, comprising
a) a monospecific bivalent antibody consisting of two full length antibody heavy chains and two full length antibody light chains whereby each chain comprises only one variable domain,
b) two variable two heavy chain single domain (VHH) antibodies, each consisting of one antibody variable and, domain, optionally,
c) peptide-linkers, connecting the C-termini of part (a) and the N-termini of part (b).

10. The antibody of claim 9, wherein the N-termini of the two heavy chain single domain (VHH) antibodies of part (b) and the C-termini of the monospecific bivalent antibody of part (a) are connected via peptide linkers.

11. The antibody of any of claims 8-10, wherein the variable domains of part (a) are capable of binding the target protein, and the variable domains of part (b) are capable of binding the VHL ligand degron of the PROTAC.

12. The antibody of any of claims 8-10, wherein the variable domains of part (b) are capable of binding the target protein, and the variable domains of part (a) are capable of binding the degron of the PROTAC.

13. The antibody of claim 11 or 12, wherein the degron of the PROTAC is a VH032 derivative of Formula I: 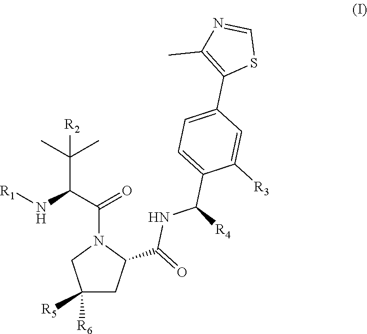
wherein
one of R₁ or R₂ is a linker connected to a warhead, with the proviso that
if R₂ is the linker, R₁ is acetyl, and
if R₁ is the linker, R₂ is methyl;
R₃ is H, OH, cyano, F, Cl, amino or methyl;
R₄ is H or methyl;
R₅, R₆ are H or OH, with the proviso that
if R₆ is H, R₅ is OH, and
if R₅ is H, R₆ is OH.

14. The antibody of claim 13, wherein
R₁ is PB-Q-(CH₂—CH₂—O)—(CH₂—CH₂—CH₂—O)ₘ—(CH₂)ₚ—(C═O)—,
wherein
PB is a protein binding warhead,
Q is NH, C═O, or absent,
n, m are, independently, 0, 1, 2, 3 or 4,
p is 0-10;
R₂ is methyl;
and R₃, R₄, R₅, and R₆ are as described in claim 13.

15. The antibody of claim 14, wherein
R₁ is PB-Q-(CH₂—CH₂—O)—(CH₂—CH₂—CH₂—O)ₘ—(CH₂)ₚ—(C═O)—,
wherein
PB is a protein binding warhead;
Q is NH, C═O, or absent;
(xi) n, m, p are 1; or
(xii) n is 3 or 4, m is 0, p is 1; or
(xiii) n is 1, m is 0, p is 2; or
(xiv) n is 2, m is 0, p is 2; or
(xv) n, m are 0, p is 6, 7, 8, 9 or 10;
R₂ is methyl;
and R₃, R₄, R₅, and R₆ are as described in claim 13.

16. The antibody of claim 13, wherein
R₁ is acetyl;
R₂ is PB—NH—(CH₂)ₚ—S—, wherein PB is a protein binding warhead,
and p is 1, 2, 3, 4, 5, or 6;
and R₃, R₄, R₅, and R₆ are as described in claim 13.

17. The antibody of claim 13, wherein the PROTAC is chosen from the PROTACs shown in FIGS. 8 (a) and 8 (b).

18. The antibody of any of claims 1, 3-17, wherein the target protein is a cell surface protein.

19. The antibody of claim 18, wherein the cell surface protein is a tumor antigen.

20. The antibody of claim 19, wherein the cell surface protein is Her2, CD33, CLL1, TROP2, NAPI2B, B7H3 or EGFR.

21. The antibody of any of claims 1-20, wherein the variable domains capable of binding the degron of the PROTAC are those of a full-length antibody, and comprise the following CDR sequences:
HC CDR1:
(SEQ ID NO: 1)
G Y S X₁ T X₂ X₃ Y;
HC CDR2:
(SEQ ID NO: 2)
I T Y S G X₄ T;
HC CDR3:
(SEQ ID NO: 3)
X₅ X₆ Y X₇ X₈ X₉ X₁₀ X₁₁ X₁₂ X₁₃ X₁₄ X₁₅;
LC CDR1:
(SEQ ID NO: 4)
Q X₁₆ X₁₇ X₁₈ X₁₉ X₂₀ X₂₁ X₂₂ X₂₃ X₂₄ Y;
LC CDR2:
(SEQ ID NO: 5)
X₂₅ X₂₆ X₂₇;
LC CDR3:
(SEQ ID NO: 6)
X₂₈ Q X₂₀ X₃₀ X₃₁ X₃₂ PY T;
wherein: X₁ is I or A; X₂ is G or N; X₃ is D or N; X₄ is G or A; X₅ is A or G; X₆ is K or Y; X₇ is G or Y; X₈ is absent or A; X₉ is absent or V; X₁₀ is absent or P; X₁₁ is D or Y; X₁₂ is G or Y; X₁₃ is G or F; X₁₄ is R or A; X₁₅ is D or H; X₁₆ is S or G; X₁₇ is L or I; X₁₈ is S or absent; X₁₉ is Y or absent; X₂₀ is S or absent; X₂₁ is D or absent, X₂₂ is G or absent; X₂₃ is N or G; X₂₄ is T or N; X₂₅ is L or Y; X₂₆ is V or A; X₂₇ is S or T, X₂₈ is V or L; X₂₉ is S or Y; X₃₀ is I or D; X₃₁ is H or E; and X₃₂ is V or Y.

22. The antibody of any of claims 1-20, wherein the variable domains capable of binding the degron of the PROTAC are those of a VHH antibody, and comprise the following CDR sequences:
CDR1:
(SEQ ID NO: 17)
G X₁ X₂ X₃ X₄ X₅ X₆ X₇;
CDR2:
(SEQ ID NO: 18)
X₃ X₃ X₁₀ X₁ X₁₂ X₁₃ X₁₄ X₁₅;
CDR3:
(SEQ ID NO: 19)
X₁₆ X₁₇ X₁₈ X₁₉ X₂₀ S X₂₁ X₂₂ X₂₃ X₂₄
X₂₅ X₂₆ X₂₇ X₂₈ X₂₉ X₃₀ X₃₁ X₃₂ X₃₃ X₃₄
X₃₅ X₃₆;
wherein: X₁ is F or R; X₂ is T, A, S or R; X₃ is L or F; X₄ is D or N; X₅ is D or T; X₆ is Y or L; X₇ is A or T; X₈ is I, N or L; X₉ is S or T; X₁₀ is S or W; X₁₁; is S or N; X₁₂ is D or G; X₁₃ is G or D; X₁₄ is S or N; X₁₅ is A, or T; X₁₆ is A, S or T; X₁₇ is A, V or I; X₁₈ is S, A, I or D; X₁₉ is T, Y, R or A; X₂₀ is R, Y or G; X₂₁ is V, S, L or T; X₂₂ is L, G, S or C; X₂₃ is S, A, C or P; X₂₄ is T, A, S or N; X₂₅ is P, I, V or D; X₂₆ is absent, V or A; X₂₇ is D, S, R, or absent; X₂₈ is V, G or P; X₂₉ is D, T, G or R; X₃₀ is Q, I, T or R; X₃₁ is V, K or R; X₃₂ is R, I or Y; X₃₃ is Y, Q, F or A; X₃₄ is V or L; X₃₅ is E, P or D; X₃₆ V, Y or A.

23. The antibody of claim 22, wherein
X₁ is F; X₂ is T or S; X₃ is L or F; X₄ is D; X₅ is D; X₆ is Y; X₇ is AT; X₈ is I; X₉ is ST; X₁₀ is S; X₁₁ is S; X₁₂ is D; X₁₃ is G; X₁₄ is S; X₁₅ is A, or T; X₁₆ is A or S; X₁₇ is V or A; X₁₈ is A or I; X₁₉ is T or Y; X₂₀ is G or R; X₂₁ is L or S; X₂₂ is C or S; X₂₃ is P or C; X₂₄ IS A or S; X₂₅ is V or D; X₂₆ is absent or V; X₂₇ is R, or absent; X₂₈ is G or P; X₂₉ is T or G; X₃₀ is Q, or I; X₃₁ is K or R; X₃₂ is R, I or Y; X₃₃ is F or A; X₃₄ is L; X₃₅ is E, or D; X₃₆ V or Y.

24. The antibody of claim 23, wherein
CDR1 is
(SEQ ID NO: 21)
GFSFDDYA
CDR2 is
(SEQ ID NO: 22)
ISSSDGST
CDR3 is
(SEQ ID NO: 23)
SAIYRLSCSVVRPTIRYALDY.

25. The antibody of claim 23, wherein
CDR1 is
(SEQ ID NO: 25)
GFTFDDYA
CDR2 is
(SEQ ID NO: 26)
ISSSDGSA
CDR3 is
(SEQ ID NO: 27)
AVATGSCPADGGQKIFLEV.

26. In vitro use of a mono-specific antibody of any preceding claim for detecting, quantifying or purifying PROTAC.

27. A complex (PAX) of a bi-specific antibody of any of claims 1, 3-25 and a PROTAC, wherein the bi-specific antibody binds to the degron of the PROTAC.

28. The complex (PAX) of claim 26, wherein the degron and the linker of the PROTAC are as described in any of claims 13-17.

29. Pharmaceutical composition, comprising the complex of claim 27 or 28, and one or more further pharmaceutically acceptable ingredients.

30. Use of the complex of claim 27 or 28 to deliver a PROTAC to a target cell, which expresses the degradation target protein.

31. Method for treating a disease by administering the complex of claim 27 or 28 to a patient in need thereof, wherein the disease benefits from the degradation of the degradation target protein of the PROTAC.

32. The complex (PAX) of claim 27 or 28 for use in treating a disease which benefits from the degradation of the degradation target protein of the PROTAC.

33. The complex (PAX) of claim 27 or 28 for use in treating a disease which benefits from the degradation of the degradation target protein of the PROTAC, wherein the PAX, is administered first, followed by a subsequent administration of the PROTAC component of the PAX alone.

34. The complex (PAX) of claim 27 or 28 for use in treating a disease which benefits from the degradation of the degradation target protein of the PROTAC, wherein the antibody component of the PAX, is administered first, and the PROTAC component of the PAX, is administered subsequently.
